(19) United States
(12) Patent Application Publication
(10) Pub. No.: US 2011/0311475 A1
(43) Pub. Date: Dec. 22, 2011

(54) MEANS AND METHOD FOR THE TREATMENT OF ANTIBODY DEFICIENCY DISEASES BASED ON IL-21 AND IL-21 VARIANTS

(76) Inventors: Stephan Borte, Leipzig (DE); Lennart Hammarström, Huddinge (SE); Ulrich Sack, Leipzig (DE)

(21) Appl. No.: 13/143,235

(22) PCT Filed: Jan. 5, 2010
(86) PCT No.: PCT/EP2010/050040

§ 371 Date: Sep. 7, 2011

(30) Foreign Application Priority Data
Jan. 5, 2009 (EP) 09150038.9

(51) Int. Cl. A61K 38/20; A61P 37/04; C07K 14/54

(52) U.S. Cl. 424/85.2; 530/351

(57) ABSTRACT

The present invention relates to an lnterleukin-21 (IL-21) variant which is capable of increasing the secretion of IgG and/or IgA antibodies in B cells and/or is capable of binding the IL-2 receptor complex and/or the IL-4 receptor complex, comprising stretches of amino acids of lnterleukin-4 (IL-4) or lnterleukin-2 (IL-2) in substitution of amino acids of IL-21. The present invention also relates to a pharmaceutical composition comprising IL-21 and/or an IL-21 variant and at least one compound selected from IgA inducing protein (IGIP), Syntenin-1, Galectin-1 and Galectin-3. The present invention further relates to a pharmaceutical composition for the treatment of a primary humoral immunodeficiency disease comprising IL-21 and/or an IL-21 variant and IL-4 and/or IL-2 and/or IGIP and/or Syntenin-1 and/or Galectin-1 and/or Galectin-3. Furthermore the present invention relates to a kit for the treatment of a primary humoral immunodeficiency disease, comprising IL-21 and/or an IL-21 variant and IL-4 and/or IL-2 and/or IGIP and/or Syntenin-1 and/or Galectin-1 and/or Galectin-3 and optionally at least one element selected from a stimulator of CD40 molecules, a ligand of the tumor necrosis superfamily, a polypeptide with human leukocyte interferon activity, a vaccine protein antigen; and a vaccine polysaccharide antigen.

The present invention relates to an Interleukin-21 (IL-21) variant which is capable of increasing the secretion of IgG and/or IgA antibodies in B cells and/or is capable of binding the IL-2 receptor complex and/or the IL-4 receptor complex, comprising stretches of amino acids of Interleukin-4 (IL-4) or Interleukin-2 (IL-2) in substitution of amino acids of IL-21. The present invention also relates to a pharmaceutical composition comprising IL-21 and/or an IL-21 variant and at least one compound selected from IgA inducing protein (IGIP), Syntenin-1, Galectin-1 and Galectin-3. The present invention further relates to a pharmaceutical composition for the treatment of a primary humoral immunodeficiency disease comprising IL-21 and/or an IL-21 variant and IL-4 and/or IL-2 and/or IGIP and/or Syntenin-1 and/or Galectin-1 and/or Galectin-3. Furthermore the present invention relates to a kit for the treatment of a primary humoral immunodeficiency disease, comprising IL-21 and/or an IL-21 variant and IL-4 and/or IL-2 and/or IGIP and/or Syntenin-1 and/or Galectin-1 and/or Galectin-3 and optionally at least one element selected from a stimulator of CD40 molecules, a ligand of the tumor necrosis superfamily, a polypeptide with human leukocyte interferon activity, a vaccine protein antigen; and a vaccine polysaccharide antigen.

Primary humoral immunodeficiency diseases are disorders resulting from inherited or spontaneous defects of the immune system. The notion comprises multiple isolated defects and combined disorders, including humoral immune deficiencies, severe combined immunodeficiencies, and disorders resulting from phagocytic and complement defects. Common variable immunodeficiency (CVID) and selective IgA deficiency (IgAD) represent the most prevalent primary immunodeficiency diseases in Caucasians. The diagnosis of CVID is inter alia based on an inability to mount protective antibody responses in the presence of normal numbers of circulating B cells and exclusion of other causes of antibody deficiency. While many individuals with IgAD are asymptomatic, some suffer from an increased susceptibility to infections of the respiratory and gastrointestinal tracts. CVID shows a highly variable clinical presentation and outcome. The clinical picture is dominated by upper and lower respiratory tract infections, leading to chronic lung disease, bronchiectasis, and eventually death. Moreover, granulomatous inflammation, gastrointestinal disorders, autoimmunity and malignancies are complicating factors in CVID.

CVID and IgAD are chiefly characterized by low or absent levels of switched immunoglobulin isotypes in general or by low or absent levels of serum IgA, respectively.

As one possible explanation for primary humoral immunodeficiency diseases problems during immunoglobuline (Ig) isotype switching are discussed. Ig isotype switching, or class switching, is a process by which B lymphocytes shift from production of IgM to one of the IgG3, IgG1, IgG2b, IgG2a, IgE, or IgA classes and subclasses in mouse or to IgG3, IgG1, IgA1, IgG2, IgG4, IgE, and IgA2 in humans (Zhang, J., et al. (2003) J. Leukocyte Biology, 73, 323-332). This process is mainly mediated by the deletional DNA recombination between the switch (S) region of the Ig heavy chain (IgH) constant region μ gene (Sμ) and one of the downstream S regions located 5′ to each IgH except for the δ gene (Zhang, J., et al. (1995) Immunoglobulin Genes, 2nd ed. (T. Honjo, F. W. Alt, eds.), Academic Press, London, 235-265). This process is known as Ig class switch recombination (CSR). Ig CSR creates a new, transcriptional unit encompassing the original variable/diversity/joining (VDJ) fragment, plus the

IgH chain to be expressed for production of a class-switched Ig isotype therefore generates a new type of Ig molecule with original antigen-binding specificity and novel effector functions associated with the IgH chain (Wang, A. C., et al. (1970) Proc. Natl. Acad. Sci. USA 66, 337-341; Cooper, M. D., et al. (1977) Cold Spring Harb. Symp. Quant. Biol. 41, 139-145). This process provides the basis for the versatile, humoral, immune functions of Ig molecules. CSR requires the participation of multiple cellular and molecular processes. The CSR process can be divided into three major steps. Transcription of a given germline IgH gene, termed Ig germline transcription, is the initial step for CSR. This process, which selectively determines the accessibility of a given IgH locus for CSR, is activated and directed by cytokine(s) and synergized by the costimulation of CD40. Ig germline transcription appears to be optimized by the 3′ Igα enhancer via its locus control region function providing for efficient germline transcription and CSR. Following Ig germline transcription, S region DNA undergoes a conformational change so that it can be served as an appropriate substrate for S region nicking and cleavage through an activation-induced cytidine deaminase (AID)-dependent mechanism. Finally, the induced double strand breaks in the S regions are appropriately processed, repaired, and ligated to join the broken ends through a general nonhomologous end-joining pathway. This final step of CSR generates a recombined chimeric S region in the chromosome, accompanying with the loop-out and deletion of the intervening DNA between the two CSR partners. Understanding CSR has significantly advanced in the past several years with progress especially occurring in the characterization of Ig germline promoters and the role of AID (Stavnezer, J. (2000) Curr. Top. Microbiol. Immunol. 245, 127-168; Honjo, T., et al. (2002) Annu. Rev. Immunol. 20, 165-196).

In the “accessibility model” of class switch recombination it has been established that cytokines direct appropriately activated B cells, such as lipopolysaccharide (LPS) and CD40 ligand (CD40L) stimulated B lymphocytes, towards switching to specific immunoglobulin isotypes (Zhang, J. et al. (1995) Immunoglobulin Genes, 2nd ed. (T. Honjo, F. W. Alt, eds.), Academic Press, London, 235-265; Stavnezer, J. (2000) Curr. Top. Microbiol. Immunol. 245, 127-168; Honjo, T. et al. (2002) Annu. Rev. Immunol. 20, 165-196; Litinskiy, M. B., et al. (2002) Nat. Immunol. 3, 822-829). For example in mouse, IL-4 preferentially directs B cell switching to IgG1 and IgE, interferon-γ to IgG2a and IgG3 and transforming growth factor-β (TGF-β) to IgA and IgG2b (Stavnezer, J. (2000) Curr. Top. Microbiol. Immunol. 245, 127-168; Snapper, C. F., et al. (1997) Immunity 6, 217-223). In humans, IL-4 drives B cell switching to IgE, IgG4 (Stavnezer, J. (2000) Curr. Top. Microbiol. Immunol. 245, 127-168; Snapper, C. F., et al. (1997) Immunity 6, 217-223), IgG3, and IgG1 (Fujieda, S. et al. (1995) J. Immunol. 155, 2318-2328), IL-10 to IgG3 and IgG1 (Defrance, T. et al. (1992) J. Exp. Med. 175, 671-682), IL-13 to IgG4 and IgE (Punnonen, J. et al. (1993) PNAS USA 90, 3730-373411), and TGF-β, vasoactive intestinal peptide, and dendritic cells to IgA (Fayette, J. et al. (1997) J. Exp. Med. 185, 1909-1918). Cytokine-driven isotype switching directly correlates with the ability of the given cytokine to selectively induce germline transcription from a specific IgH locus, and the induced germline transcription precedes CSR. It is correspondingly assumed that a given cytokine, by inducing transcription through a specific IgH locus, opens the locus so as to be “accessible” to the putative, pre-existing Ig-CSR machinery for CSR.

Cytokines are a large family of more than 100 proteins that function as mediators involved in essentially all biological processes. They have been found to be important rate-limiting signals, and blocking some cytokines yields effective therapeutics. Cytokines are low-MW proteins that usually act at short range between neighboring cells. These molecules, previously also termed interleukins, interferons, growth factors, and TNFs, among other designations, are involved in essentially every important biological process, from cell proliferation to inflammation, immunity, migration, fibrosis, repair, and angiogenesis. As these molecules and their associated receptors provide key signals for important processes, it is not surprising that abnormalities in cytokines, their receptors, and the signaling pathways that they initiate are involved in a wide variety of diseases.

The field of cytokines came of age in the late 1970s with the introduction of molecular biological approaches that resulted first in the cloning of IFNs, initially IFN-β and IFN-α. By the mid-1980s, there was a plethora of well-defined cytokines and cytokine receptors that could be unambiguously studied, using molecular tools, such as cDNA probes, and antibodies that had been produced to recognize the pure recombinant proteins.

The action of cytokines may be autocrine, paracrine, and endocrine. Cytokines are critical to the development and functioning of both the innate and adaptive immune response. They are often secreted by immune cells that have encountered a pathogen, thereby activating and recruiting further immune cells to increase the system's response to the pathogen.

A commonly accepted functional classification of cytokines divides immunological cytokines into two groups: those that enhance cytokine responses, which are termed type 1 cytokines. This group comprises, for example, IFN-γ and TGF-β. The other group encompasses those cytokines which favor antibody responses and is called type 2 groups. The group comprises, for example, IL-4, IL-10 or IL-13.

An alternative structural classification of cytokines distinguishes between the four α-helix bundle family, the IL-1 family and the IL-17 family. The members of the four α-helix bundle family have three-dimensional structures with four bundles of α-helices. This family in turn is divided into three sub-families, i.e. the IL-2 subfamily, the interferon (IFN) subfamily and the IL-10 subfamily. The first of these three subfamilies is the largest. It contains several non-immunological cytokines including erythropoietin (EPO) and thrombopoietin (THPO). Four α-helix bundle cytokines can be grouped into long-chain and short-chain cytokines. The IL-1 family primarily includes IL-1 and IL-18, whereas the members of the IL-17 family have a specific effect in promoting proliferation of T-cells that cause cytotoxic effects.

Interleukin-21 (IL-21) is a recently discovered cytokine whose pleiotropic effects on the immune system are just beginning to be examined. IL-21 was functionally cloned as the ligand of an orphan receptor, IL-21R, using Ba-F3 cells transfected with a chimeric IL-21R that could induce proliferation in response to binding of the correct ligand (Parrish-Novak J, et al. (2000) Nature; 408, 57-63). IL-21 was identified as a four-helix-bundle cytokine that is most homologous to IL-15 and also has significant homology to IL-2 and IL-4. It is of the γc family that is expressed exclusively by activated CD4+ Th2 cells. Human Interleukin-21 (IL-21) is a four-α-helix-bundle cytokine of the γc family that is expressed exclusively by activated CD4+ Th2 cells.

IL-21 exerts variable and sometimes contrasting effects on NK, T and B cells. Among its effects on B cells, IL-21 induces proliferation or apoptosis in a context-dependant manner, and production of antigen-specific antibodies. The human IL-21 gene was mapped to 4q26-q27 and is only 180 kb away from the IL-2 gene while the IL-15 gene is more distal at 4q31 (Parrish-Novak J, et al. (2000) Nature; 408, 57-63). Also, the exon and intron structures of the IL-2 and IL-21 genes are very similar, suggesting that because of their proximity to each other and their similar genetic organization these two genes may have arisen by gene duplication (Mehta et al. (2004) Immunological Reviews 202, 84-95). Human and murine IL-21 are 57% identical at the amino acid level and have an even greater conservation of structural components. Interestingly, IL-21 receptor (IL-21R) deficient mice show no obvious developmental defects, but markedly diminished IgG1, IgG2 and IgG3 levels while IgE is elevated (Ozaki K, et al. (2002) Science 298 (5598), 1630-4; Kasaian MT, et al. (2002) Immunity 16 (4), 559-69).

Thus, although the elucidation of mechanistic interrelationships and connections prevailing during the production of humoral immune responses, in particular in the context of Ig isotype switching has reached an advanced stage, the main and by far most typical treatment of primary antibody deficiencies like CVID and IgAD is still a replacement immunoglobulin therapy. The purpose of this therapeutical approach is the restoration of physiological levels of IgG or IgA in the patient's blood and, thus, the alleviation of the severity of infections. However, this treatment scheme does not alter or remedy the underlying molecular problem, but only aims at an alleviation of concomitant symptoms.

Therefore, there is a need for the provision of a new and effective treatment perspective for the treatment of primary humoral immunodeficiency diseases are disorders resulting from inherited defects of the immune system, in particular for common variable immunodeficiency (CVID) and selective IgA deficiency (IgAD).

The present invention addresses this need and provides an IL-21 variant which is capable of increasing the secretion of IgG and/or IgA antibodies in B cells, comprising stretches of amino acids of IL-4 or IL-2 in substitution of amino acids of IL-21. These variants and/or IL-21 in combination with IL-4 and/or IL-2 may effectively be used for the treatment of a primary humoral immunodeficiency disease. A kit, comprising IL-21 and/or an IL-21 variant and IL-4 and/or IL-2 and optionally at least one element selected from a stimulator of CD40 molecules, a ligand of the tumor necrosis superfamily, a polypeptide with human leukocyte interferon activity, a vaccine protein antigen; and a vaccine polysaccharide antigen may further be used for the treatment of a primary humoral immunodeficiency disease.

The inventors surprisingly found that IL-21 variants comprising stretches of amino acids of IL-4 or IL-2 in substitution of amino acids of IL-21 are capable of increasing the secretion of IgG and/or IgA antibodies in B cells and/or are capable of binding the the IL-2 receptor complex and/or the IL-4 receptor complex. Moreover, the present inventors surprisingly found that a pharmaceutical composition comprising such IL-21 variants and/or IL-21 and at least one compound selected from the group of IL-4, IL-2, IgA inducing protein (IGIP), Syntenin-1, Galectin-1 and Galectin-3 can effectively be used for medical applications, in particular for the treatment of a primary humoral immunodeficiency disease, e.g. common variable immunodeficiency (CVID) or selective IgA deficiency (IgAD). This new therapeutic approach offers the considerable advantage of rendering antibody replacement or substitution treatments superfluous since the patients treated according to the invention's approach are capable of producing the lacking antibody isotypes de novo and in vivo. Accordingly, it is no longer necessary to obtain, purify and store antibodies in large quantities for replacement or substitution treatments. Moreover, the treatment schemes are drastically simplified and can become more flexible. What is more, the new therapeutic approach of the present invention additionally allows the induction of IgA and/or IgG antibody production in the context of vaccination schemes, a scenario which would not be possible during antibody replacement or substitution treatments. Thus, the new therapeutic approach of the present invention allows an advantageous antigen-dependent antibody induction, which was hitherto barely feasible.

In a preferred embodiment of the present invention the IL-21 variant comprises between about 10 to 60% of the helical portions of IL-4 as defined in SEQ ID NO: 2. In a more preferred embodiment of the present invention the IL-21 variant also comprises interhelical portions of IL-4 as defined in SEQ ID NO: 2.

In another preferred embodiment of the present invention the IL-21 variant comprises between about 10 to 65% of the helical portions of IL-2 as defined in SEQ ID NO: 3. In a more preferred embodiment of the present invention the IL-21 variant also comprises interhelical portions of IL-2 as defined in SEQ ID NO: 3.

In a further aspect the present invention relates to a pharmaceutical composition comprising IL-21 and/or the IL-21 variant as mentioned above, and at least one compound selected from the group consisting of IgA inducing protein (IGIP), Syntenin-1, Galectin-1 and Galectin-3.

In a further aspect the present invention relates to a pharmaceutical composition for the treatment of a primary humoral immunodeficiency disease, comprising IL-21 and/or the IL-21 variant as mentioned above, and at least one compound selected from the group consisting of IL-4, IL-2, IgA inducing protein (IGIP), Syntenin-1, Galectin-1 and Galectin-3.

In preferred embodiment of the present invention the pharmaceutical composition comprises IL-21 and/or the IL-21 variant as mentioned above, and IL-4 and IL-2.

In another aspect the present invention relates to the use of IL-21 and/or the IL-21 variant as mentioned above, and at least one compound selected from the group consisting of IL-4, IL-2, IgA inducing protein (IGIP), Syntenin-1, Galectin-1 and Galectin-3 for the preparation of a pharmaceutical composition for the treatment of a primary humoral immunodeficiency disease.

In yet another aspect the present invention relates to a kit for the treatment of a primary humoral immunodeficiency disease, comprising:
(i) IL-21 and/or the IL-21 variant as mentioned above; and
(ii) IL-4 and/or IL-2 and/or
(iii) IgA inducing protein (IGIP) and/or Syntenin-1 and/or Galectin-1 and/or Galectin-3.

In a preferred embodiment of the present invention the kit also comprises at least one element selected from:
(iv) a stimulator of CD40 molecules;
(v) a ligand of the tumor necrosis superfamily;
(vi) a polypeptide with human leukocyte interferon activity;
(vii) a vaccine protein antigen; and
(viii) a vaccine polysaccharide antigen.

In particularly preferred embodiment of the present invention the kit as mentioned above comprises a stimulator of CD40 molecules selected from the group consisting of an anti-CD40 antibody, a CD40 ligand (CD40L) and C4BP.

In a further, particularly preferred embodiment of the present invention the kit as mentioned above comprises a ligand of the tumor necrosis superfamily selected from the group consisting of BAFF and LIGHT.

In a further, particularly preferred embodiment of the present invention the kit as mentioned above comprises a polypeptide with human leukocyte interferon activity like Interferon-α (IFN-α).

In a further preferred embodiment of the present invention the pharmaceutical composition or kit as mentioned above comprises IL-21 or an IL-21 variant as mentioned above and IL-4 in a ratio between about 5:1 and 25:1. In a more preferred embodiment the ratio between IL-21 or an IL-21 variant as mentioned above and IL-4 in the pharmaceutical composition or kit is about 20:1.

In a further preferred embodiment of the present invention the pharmaceutical composition or kit as mentioned above comprises IL-21 or an IL-21 variant as mentioned above and IL-2 in a ratio between about 5:1 and 20:1. In a more preferred embodiment the ratio between IL-21 or an IL-21 variant as mentioned above and IL-2 in the pharmaceutical composition or kit is about 15:1.

In a further preferred embodiment of the present invention the pharmaceutical composition as mentioned above further comprises at least one stimulator of CD40 molecules. In a more preferred embodiment of the present invention the pharmaceutical composition as mentioned above comprises a stimulator of CD40 molecules selected from the group consisting of an anti-CD40 antibody, CD40 ligand (CD40L) and C4BP.

In yet another preferred embodiment of the present invention the pharmaceutical composition as mentioned above further comprises at least one ligand of the tumor necrosis factor superfamily and/or at least one polypeptide with human leukocyte interferon activity. In a more preferred embodiment of the present invention the pharmaceutical composition as mentioned above comprises a ligand of the tumor necrosis factor superfamily selected from the group consisting BAFF or LIGHT and/or a polypeptide with human leukocyte interferon activity like Interferon-α (IFN-α).

In further preferred embodiment of the present invention the pharmaceutical composition as mentioned above further comprises at least one vaccine protein antigen and/or at least one vaccine polysaccharide antigen.

In another preferred embodiment of the present invention the kit as mentioned above is administered such that the interim between the administration of IL-21 and/or the IL-21 variant as mentioned above on the one hand and IL-4 and/or IL-2 on the other hand is between about 1 minute and 12 hours.

In another preferred embodiment of the present invention the kit as mentioned above is administered such that the interim between the administration of IL-21 and/or the IL-21 variant as mentioned above and IL-4 and/or IL-2 on the one hand and the administration of any of (a) a stimulator of CD40 molecules, (b) a ligand of the tumor necrosis superfamily, (c) a polypeptide with human leukocyte interferon activity, (d) a vaccine protein antigen; and (e) a vaccine polysaccharide antigen on the other hand is between about 12 hours and 72 hours.

In a more preferred embodiment of the present invention the stimulator of CD40 molecules as mentioned above is selected from the group consisting of an anti-CD40 antibody, a CD40 ligand (CD40L) and C4BP.

In a more preferred embodiment of the present invention the ligand of the tumor necrosis superfamily as mentioned above is selected from the group consisting of BAFF and LIGHT.

In a more preferred embodiment of the present invention the polypeptide with human leukocyte interferon activity as mentioned above is Interferon-α (IFN-α).

In a further aspect the present invention relates to a live carrier expressing IL-21 or an IL-21 variant as defined herein above and at least one element selected from the group consisting of IL-4, IL-2, IgA inducing protein (IGIP), Syntenin-1, Galectin-1 and Galectin-3.

In a preferred embodiment of the present invention the live carrier also expresses at least one element selected from:
(i) a stimulator of CD40 molecules;
(ii) a ligand of the tumor necrosis superfamily;
(iii) a polypeptide with human leukocyte interferon activity; and
(iv) a vaccine protein antigen.

In particularly preferred embodiment of the present invention the live carrier as mentioned above expresses a stimulator of CD40 molecules selected from the group consisting of an anti-CD40 antibody, a CD40 ligand (CD40L) and C4BP.

In a further, particularly preferred embodiment of the present invention the live carrier as mentioned above expresses a ligand of the tumor necrosis superfamily selected from the group consisting of BAFF and LIGHT.

In a further, particularly preferred embodiment of the present invention the live carrier as mentioned above expresses a polypeptide with human leukocyte interferon activity like Interferon-α (IFN-α).

In a further preferred embodiment of the present invention the live carrier as mentioned above is for the treatment of a primary humoral immunodeficiency disease.

In another preferred embodiment of the present invention the pharmaceutical composition, use, kit or live carrier as mentioned above is for the treatment of a disease involving a reduction in the level of secreted IgG and/or IgA antibodies. In another preferred embodiment of the present invention the pharmaceutical composition, use, kit or live carrier as mentioned above is for the treatment of selective deficiency of IgA (IgAD), common variable immunodeficiency (CVID), selective deficiency of IgG subclasses (IgGsD), immunodeficiency with increased IgM (hyper-IgM-syndrome) or X-linked agammaglobulinaemia.

These and other characteristics, features and objectives of the present invention will become apparent from the following detailed description, taken in conjunction with the accompanying figures and examples, which demonstrate by way of illustration the principles of the invention.

The description is given for the sake of example only, without limiting the scope of the invention.

### DESCRIPTION OF THE FIGURES

FIG. 1 shows the structure of Interleukin-2, Interleukin-4 and Interleukin-21 displaying helices as dark-grey pleated sheet structures and interhelical portions as light-grey lines.

FIG. 2 gives a schematic overview over the interaction of different interleukins to their cognate receptors. The figure shows the interaction between IL-2 and IL-2Rβ, IL-2Rα and γc, the interaction between IL-4 and IL-4R and γc and the interaction between IL-21 and IL-21R, and γc.

FIG. 3 shows the induction of IgG production of PBMC from 5 randomly chosen healthy donors presented as mean values. Units of secreted IgG were measured in a virtual unit that is equivalent to the surface in (0.01 mm)² multiplied by the intensity of a particular spot in ELISPOT analysis. In FIG. 3A 1×10⁶ PBMC were stimulated for 5 days with either 10 or 100 ng/ml of IL-4, IL-10 or IL-21 alone or in combination with 2 μ/ml anti-human CD40 mAb. Subsequently, 1×10⁵ PBMC were subjected to ELISPOT assay for 20 h of incubation. In FIG. 3B 1×10⁶ PBMC were stimulated for 5 days with 2 μ/ml of anti-human CD40 mAb and 0.5 ng/ml of either IL-2, IL-4, IL-6, IL-7, IL-15 or IL-10 alone (grey bars) or in combination with 10 ng/ml of IL-21 (black bars). 5×10⁴ PBMC were subjected to ELISPOT assay for 20 h of incubation.

FIG. 4 shows the expression of CD27, CD138 and surface IgD, IgG and IgA on CD19⁺ lymphogated cells in a representative healthy individual. Cell surface expression of these markers is represented on a four-decade log scale as dot plots of correlated x-axis and y-axis fluorescence. FCM analysis was performed at days 0, 3, 5 and 7 with PBMC cultured in the presence of IL-21 [10 ng/ml], IL-4 [0.5 ng/ml] and anti-human CD40 mAb [2 μg/ml]. In FIG. 4A quadrant markers were positioned to include naïve mature B cells (UL), natural effector B cells (UR), and IgD⁻ memory B cells (LR). The circle tags a population of CD27^{high} IgD⁻ B cells. In FIGS. 4B and D quadrant markers were positioned to separate CD138^{high} plasma cells (UL) from sIgA⁺ B cells (LR). In FIGS. 4C and E quadrant markers were positioned to separate CD138^{high} plasma cells (UL) from sIgG^{high} B cells (LR).

FIG. 5 shows the effect of cytokine and anti-CD40 stimulation on IgG and IgA production in PBMC from 32 patients with CVID (FIGS. 5A and B) and 10 individuals with IgAD (FIG. 5C). 1×10⁶ PBMC were stimulated for 5 days with cytokines (IL-10 and IL-21 at 10 ng/ml, IL-4 at 0.5 ng/ml) plus anti-human CD40 mAb at 2 μg/ml, if stated. Subsequently, 1×10⁵ PBMC were subjected to ELISPOT assay for 20 h of incubation. Units of secreted IgG (FIG. 5A) and IgA (FIGS. 5B and C) were measured in a virtual unit that is equivalent to the surface in (0.01 mm)² multiplied by the intensity of a particular spot in ELISPOT analysis. To allow better interpretability of the effect of cytokine and anti-CD40 stimulation on IgG and IgA production between patients with a primary humoral immunodeficiency disease and healthy controls, FIGS. 5 D-F compare results from the 32 patients with CVID (FIGS. 5D and E, filled circles) and 10 individuals with IgAD (FIG. 5F) with 22 healthy controls (open circles). 1×10⁶ PBMC were stimulated for 5 days with cytokines (IL-10 and IL-21 at 10 ng/ml, IL-4 at 0.5 ng/ml) plus anti-human CD40 mAb at 2 μg/ml, if stated. Subsequently, 5×10⁴ and 1×10⁵ PBMC were subjected to ELISPOT assay for 20 hrs of incubation. Units of secreted IgG (FIG. 5D) and IgA (FIGS. 5E and F), presented on a logarithmic scale, were measured in a virtual unit that is equivalent to the surface in (0.01 mm)² multiplied by the intensity of a particular spot in ELISPOT analysis.

FIG. 6 depicts the expression of AID mRNA, the rate of Iγ-Cγ or Iα-Cα germline transcription and the presence of Iγ-Cμ or Iα-Cμ switch circle transcripts in PBMC from 15 patients with CVID (FIGS. 6A and B) and 10 individuals with IgAD (FIG. 6C) at day 3 of cell culture with IL-21 [10 ng/ml], IL-4 [0.5 ng/ml] and anti-human CD40 mAb [2 μg/ml]. The diameter of shown dots correlates proportionally with units of secreted IgG (A) or IgA (B and C), detected in 20 h of ELISPOT assay at day 5 of culture using 5×10⁴ PBMC.

FIG. 7 shows the expression of CD27, CD138 and surface IgD, IgG and IgA on CD19⁺ lymphogated cells in representative individuals with CVID (FIGS. 7A, C, E and G) or IgAD (FIGS. 7B, D, F and H). Cell surface expression of these markers is represented on a four-decade log scale as dot plots of correlated x-axis and y-axis fluorescence. FCM analysis was performed at days 0, 3, 5 (and 7), with PBMC cultured in the presence of IL-21 [10 ng/ml], IL-4 [0.5 ng/ml] and anti-human CD40 mAb [2 μg/ml]. In FIGS. 7A, B, E and F quadrant markers were positioned to include naïve mature B cells (UL), natural effector B cells (UR), and IgD⁻ memory B cells (LR). The circle tags a population of CD27^{high} IgD⁻ B cells. In FIGS. 7C and G quadrant markers were positioned to separate CD138^{high} plasma cells (UL) from sIgG^{high} B cells (LR). In FIGS. 7D and H quadrant markers were positioned to separate CD138^{high} plasma cells (UL) from sIgA⁺ B cells (LR).

FIG. 8 depicts the identification of IgG and IgA producing cell populations in one individual with CVID (FIG. 8A) and IgAD (FIG. 8B). Expression of CD138 and surface IgG or IgA on CD19⁺ lymphogated cells was analysed prior and post magnetic separation of CD138⁺ plasma cells from PBMC cultured in the presence of IL-21 [10 ng/ml], IL-4 [0.5 ng/ml] and anti-human CD40 mAb [2 pg/ml] for 5 days. Cell surface expression of these markers is represented on a four-decade log scale as dot plots of correlated x-axis and y-axis fluorescence. Images of ELISPOT assay were taken following 20 h of incubation of 5×10⁴ PBMC for IgG production (FIG. 8A) or 1×10⁶ PBMC for IgA production (FIG. 8B). Effects of AID-silencing on the presence of surface IgG⁺ B cells and production of IgG in PBMC from 8 patients with CVID are indicated in FIG. 8C. AID expression in control samples was considered 100% expression level, while samples containing no RNA were treated as blank values (0% expression level). Numbers of sIgG⁺ B cells were detected using FCM analysis of IgG surface expression on CD19⁺ lymphogated cells at day 5 of PBMC culture. IgG production was detected at day 5 of culture during 20 hrs of ELISPOT using 4×10⁴ PBMC. Units of secreted IgG were measured in a virtual unit that is equivalent to the surface in (0.01 mm)² multiplied by the intensity of a particular spot in ELISPOT analysis.

FIG. 9 shows the expression of Interleukin-21 and Interleukin-21 receptor mRNA, analysed in PBMC from 30 patients with CVID and 22 healthy individuals following stimulation of 1×10⁶ cells with anti-human CD3 mAb for 14 hrs. The ends of the boxes define the 25th and 75th percentiles, with a line at the median and error bars defining the 10th and 90th percentiles. These results corroborate that the production of IL-21 and IL-21 receptor mRNA in T cells of patients with CVID is functional.

FIG. 10 depicts the induction of tetanus and diphtheria toxoid-specific IgG in 4 patients with CVID (black dots) and in 4 healthy individuals (white dots). Culture supernatants of 2×10⁶ PBMC, stimulated with IL-21 [10 ng/ml], IL-4 [0.5 ng/ml] and anti-human CD40 mAb [2 μg/ml], were analyzed with commercial toxoid-specific IgG ELISAs at day 7 of culture. For each sample 3 replicates were analyzed and mean values were created that disregarded outliers. Dotted base lines represent assay specific detection limits.

FIG. 11 depicts the expression of CD138 and surface IgG or IgA on CD19+ lymphogated cells in representative patients with CVID or in healthy individuals. Cell surface expression of these markers is represented on a four-decade log scale as dot plots of correlated x-axis and y-axis fluorescence. FCM analysis was performed at day 7 with PBMC cultured in the presence of IL-21 [10 ng/ml], IL-4 [0.5 ng/ml] and anti-human CD40 mAb [2 μg/ml] alone or in combination with tetanus or diphtheria toxoid. Quadrant markers were positioned to separate CD138^{high} plasma cells (UL) from sIgG^{high} or sIgA⁺ B cells (LR).

FIG. 12 shows the effect of several IL-21 variants on IgG and IgA production of CD19⁺ purified B cells from one healthy control and two patients with CVID. B cells were stimulated for 5 days with IL-21 or IL-21 variants at 10 ng/ml plus anti-human CD40 mAb at 2 μg/ml. Subsequently, 1×10⁴ B cells were subjected to ELISPOT assay for 20 h of incubation. Units of secreted IgG (FIG. 12A) and IgA (FIGS. 12B and C) were measured in a virtual unit that is equivalent to the surface in (0.01 mm)² multiplied by the intensity of a particular spot in ELISPOT analysis.

FIG. 13 shows the induction of IgG and IgA production of PBMC (A) or purified CD19⁺ B cells (B) from a CVID patient in terms of representative results out of 5 experiments. Units of secreted IgG and IgA were measured in a virtual unit that is equivalent to the surface in (0.01 mm)² multiplied by the intensity of a particular spot in ELISPOT analysis. (A) 5×10⁵ PBMC were stimulated for 5 days with either 50 or 500 ng/ml of Galectin-1 or Galectin-3 alone or in combination with 10 ng/ml of IL-21. Subsequently, 2.5×10⁵ PBMC were subjected to ELISPOT assay for 20 h of incubation. (B) 2×10⁵ CD19⁺ B cells were stimulated for 5 days with either 500 ng/ml of Galectin-1 or Galectin-3 alone or in combination with 100 ng/ml of the IL-21/IL-4 hybrid. Subsequently, 5×10⁴ CD19⁺ B cells were subjected to ELISPOT assay for 20 h of incubation.

FIG. 14 shows the induction of IgA production of purified CD19⁺ B cells from a CVID patient in terms of representative results out of 5 experiments. The photos depict the results of ELISPOT assays, whilst every dark spot represents a single IgA-producing B cell. 4×10⁵ CD19⁺ B cells were stimulated for 7 days with either 250 ng/ml of IGIP or Syntenin-1 alone or in combination with 100 ng/ml of the IL-21/IL-4 hybrid. Subsequently, 2×10⁵ CD19⁺ B cells were subjected to ELISPOT assay for 20 h of incubation.

FIG. 15 shows the induction of IgG and IgA production of PBMC from a CVID patient in terms of representative results out of 5 experiments. The photos depict the results of ELISPOT assays, whilst every dark spot represents a single IgG⁻ or IgA-producing cell. PBMC were co-cultured for 7 days with lactobacilli expressing surface-anchored CD40L, IL-21 cleaved, “Chim-hIL-21/4”, IL-21/IL-2 hybrid or IL-21/IL-4 hybrid protein. The ratio between lactobacilli and PBMC was 10:1. Subsequently, 2.5×10⁵ PBMC were subjected to ELISPOT assay for 20 h of incubation.

FIG. 16 shows the induction of IgA production of PBMC from two randomly-selected buffy coat donors with selective IgA-deficiency. The photos depict the results of ELISPOT assays, whilst every dark spot represents a single IgA-producing B cell. 5×10⁵ PBMC were stimulated for 7 days with either 50 ng/ml of IL-21 cleaved, “Chim-hIL-21/4”, IL-21/IL-2 hybrid, or IL-21/IL-4 hybrid in combination with 2 μg/ml anti-human CD40 mAb (Mabtech AB, Stockholm, Sweden). Subsequently, 2.5×10⁵ PBMC were subjected to ELISPOT assay for 20 h of incubation.

FIG. 17 shows the induction of IgA production of PBMC from a representative buffy coat donor with selective IgA-deficiency. The photos depict the results of ELISPOT assays, whilst every dark spot represents a single IgA-producing B cell. 5×10⁵ PBMC were stimulated for 7 days with either 100 ng/ml of IL-21 cleaved or 50 ng/ml “Chim-hIL-21/4” or IL-21/IL-4 hybrid in combination with 2 μg/ml anti-human CD40 mAb (Mabtech AB, Stockholm, Sweden). Subsequently, 2.5×10⁵ PBMC were subjected to ELISPOT assay for 20 h of incubation.

FIG. 18 shows results from a comparative structural analysis of IL-21, IL-4, and IL-2, specifying structural details and functional epitopes and regions that are important for the interaction of IL-21, IL-4, and IL-2 with the common γ-chain and their specific receptors.

FIG. 19 shows results from a bead-based immunoassay to detection and calculate the extent of receptor-interaction of IL-21 variant proteins. FIG. 19A shows that protein-coated beads can be securely detected by flow cytometry based on forward- and side-scatter analysis. In FIG. 19B the PE fluorescence of the color-coded beads is presented versus DyLight 649 fluorescence (APC channel) borne by receptor-chimera complexes, demonstrating that the extent of bound receptor-chimera-complex can be detected and analyzed by flow cytometry. FIG. 19C compares the ability of cleaved IL-21, “Chim-hIL-21/4”, IL-21/IL-2 hybrid protein and IL-21/IL-4 hybrid protein to bind the proprietary IL-2Rβ subunit, clearly indicating that only the IL-21/IL-2 hybrid protein shows a concentration-dependence of IL-2Rβ interaction, as intended by the design of this protein. In FIG. 19D the ability of cleaved IL-21, “Chim-hIL-21/4”, IL-21/IL-2 hybrid protein and IL-21/IL-4 hybrid protein to bind the proprietary IL-4Rα subunit is compared with each other. Whereas the “Chim-hIL-21/4” protein only allows minor IL-4Rα binding, a IL-21/IL-4 hybrid protein of the present invention shows a concentration-dependence of IL-4Rα interaction, as intended by the design of this type of protein.

### DETAILED DESCRIPTION OF THE INVENTION

The inventors have found that IL-21 variants comprising stretches of amino acids of IL-4 or IL-2 in substitution of amino acids of IL-21 are capable of increasing the secretion of IgG and/or IgA antibodies in B cells and/or are capable of binding the IL-2 receptor complex and/or the IL-4 receptor complex and that by using such variants and/or IL-21 and additionally IL-4, IL-2, IgA inducing protein (IGIP), Syntenin-1, Galectin-1 or Galectin-3 a primary humoral immunodeficiency disease, e.g. common variable immunodeficiency (CVID) or selective IgA deficiency (IgAD) can effectively be treated.

Although the present invention will be described with respect to particular embodiments, this description is not to be construed in a limiting sense.

Before describing in detail exemplary embodiments of the present invention, definitions important for understanding the present invention are given.

It must be noted that as used herein and in the appended claims, the singular forms “a”, “an”, and “the”, include plural referents unless the context clearly indicates otherwise. Thus, for example, reference to “a polypeptide” includes one or more of such polypeptides, and reference to “the method” includes reference to equivalent steps and methods known to those of ordinary skill in the art that could be modified or substituted for the methods described herein.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” or “(i)”, “(ii)”, “(iii)”, “(iv)”, “(v)”, “(vi)”, “(vii)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” or “(i)”, “(ii)”, “(iii)”, “(iv)”, “(v)”, “(vi)”, “(vii)” etc. relate to steps of a method or use there is no time or time interval coherence between the steps, i.e. the steps may be carried out simultaneously or there may be time intervals of seconds, minutes, hours, days, weeks, months or even years between such steps, unless otherwise indicated in the application or claims as set forth herein above or below.

It is to be understood that this invention is not limited to the particular methodology, protocols, proteins, bacteria, vectors, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Köibl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

As has been set out above, the present invention concerns in one aspect an IL-21 variant, wherein said IL-21 variant is capable of increasing the secretion of IgG and/or IgA antibodies in B cells and/or is capable of binding the IL-2 receptor complex and/or the IL-4 receptor complex, said IL-21 variant comprising stretches of amino acids of IL-4 or IL-2 in substitution of amino acids of IL-21 as defined in SEQ ID NO: 1.

The term “Interleukin-21” or “IL-21” refers to a human Interleukin having the polypeptide sequence as defined in SEQ ID NO: 1. The term, thus, relates to a mature, processed, cleaved or secreted version of IL-21, which has been optimized for expression in mammal cells, as depicted in SEQ ID NO:1. For certain purposes or uses of the invention, e.g. for the expression of the protein in vivo etc. an Interleukin-21 may additionally also be understood as comprising a signal sequence, preferably of additional 29 amino acids. This type of IL-21 is termed “precursor IL-21” and defined in SEQ ID NO: 4. Preferably, a “precursor IL-21” refers to a protein capable of being directed to the ER, secretory vesicles, or the extracellular space as a result of said signal sequence. If the IL-21 precursor is released into the extracellular space, the IL-21 precursor can undergo extracellular processing to produce “IL-21” or a “mature IL-21” or a “processed IL-21”, or a “cleaved IL-21”, or a “secreted IL-21” protein. Release into the extracellular space can occur by many mechanisms, including exocytosis and proteolytic cleavage. The terms “IL-21”, “mature IL-21”, “processed IL-21”, “cleaved IL-21” and “secreted IL-21” are used herein as synonyms and are to be understood, for the purpose of the present invention, as functionally equivalent. In specific embodiments of the present invention, e.g. in the context of the expression of the protein or variant in bacterial systems, the term “Interleukin-21” or “IL-21” may also relate to a sequence of IL-21 as depicted in SEQ ID NO:1 or derived therefrom as defined herein above or below, wherein at the N-terminus one additional amino acid is added that functions as a bacterial translation initiator. Preferably, the amino acid methionine may be added. A corresponding sequence is depicted in SEQ ID NO: 10.

The term “IL-21 variant” refers to an IL-21 protein, which is derived from human IL-21, preferably from an IL-21 having the polypeptide sequence as defined in SEQ ID NO:1 or SEQ ID NO:4 and being capable of increasing the secretion of IgG and/or IgA antibodies in B cells and/or being capable of binding the IL-2 receptor complex and/or the IL-4 receptor complex.

The term “capable of increasing the secretion of IgG and/or IgA antibodies in B cells” means that an IL-21 protein or IL-21 variant can enhance or raise the emission or throw-off of antibodies of the isotypes IgG or IgA, or of the isotypes IgG and IgA, preferably of at least one of the subclasses IgG1, IgG2, IgG3, IgG4, IgA1 or IgA2 by B-type lymphocytes. More preferably, the term means that an IL-21 protein or IL-21 variant can enhance or raise the emission or throw-off of antibodies of the isotypes IgG or IgA, or of the isotypes IgG and IgA, or of at least one of the subclasses IgG1, IgG2, IgG3, IgG4, IgA1 or IgA2 by B-type lymphocytes in comparison to the IL-21 protein as defined in SEQ ID NO:1 when tested under otherwise identical conditions.

The term “B-type lymphocyte” relates to cells expressing marker proteins CD19 and/or CD38 and/or CD138. Typically, B-type lymphocytes comprise immature or transitional B cells, mature naive B cell, B1- and B2-B cells, marginal zone B cells, follicular centroblast and centrozyte B cells, memory B cells, and terminally differentiated plasma B cells expressing marker protein CD38. Preferably, B-type lymphocytes are understood as plasma B cells. The term “plasma B cell” relates to terminally differentiated B-cells, which typically express marker proteins CD38, CD138 and/or plasma cell antigen-1. The capability of IL-21 proteins or IL-21 variants to increase the secretion of antibodies of the isotypes IgG or IgA, or of the isotypes IgG and IgA, preferably of at least one of the subclasses IgG1, IgG2, IgG3, IgG4, IgA1 or IgA2, by B-type lymphocytes can be tested and determined by any suitable methods known to the person skilled in the art. Preferably, the capability of IL-21 proteins or IL-21 variants to increase the secretion of antibodies of the isotypes IgG or IgA, or of the isotypes IgG and IgA, preferably of at least one of the subclasses IgG1, IgG2, IgG3, IgG4, IgA1 or IgA2, by B-type lymphocytes can be tested by a method based on Czerkinsky C C et al. (1983). J Immunol Methods 65 (1-2), 109-21.

A typical method to be employed in the context of the present invention may comprise, as a first step, the obtainment of human peripheral blood mononuclear cells (PBMC) from peripheral venous blood by Ficoll separation; preferably according to Kreher C R, et al. (2003) J Immunol Methods 278 (1-2), 79-93. The cells may be obtained for the purpose of the present procedure only from one patient type, e.g. a healthy individual, or a patient with an immunological disorder like CVID, preferably only from one individual patient. More preferably, the cells may be obtained only from a healthy patient type. The term “healthy” means that the patient is not afflicted with an immunological disorder, in particular not with CVID or IgAD. It is further preferred that the cells obtained from a healthy patient spontaneously produce no or only marginal amounts of IgG or IgA if unstimulated. Such a behavior may be tested by methods known to the person skilled in the art, e.g. based on assays described herein above or below. The term “marginal amounts” means that in an ELISPOT assay as described herein a limit of overall 200 units equivalent to the surface in (0.01 mm)² multiplied by the intensity of the counted spots may not be surpassed. Alternatively, commercially available cells may be used, which have been normalized with regard to the production of IgA and/or IgG.

Subsequently, CD19 positive B cells and CD138 positive plasma cells may be isolated from PBMC by any suitable method, e.g. by nanoparticle-based immunomagnetic cell selection, preferably with selection kits (e.g. commercially available kits of Miltenyi Biotec Inc., STEMCELL Technologies Inc. etc.). In a next step, the PBMC or the selected B-type lymphocytes may be cultured in any suitable culture medium as known to the person skilled in the art, e.g. in Iscove's Modified Dulbecco's medium (IMDM). For the culturing an amount of 1×10⁴ to 2×10⁶ cells (PBMC or selected B type lymphocytes) may be used. The amount of cells may be determined by any suitable means known to the person skilled in the art, e.g. by cell counting devices or machines, microscopy determination etc.

Preferably, the medium may comprise L-Alanyl-L-Glutamine, HEPES, Penicillin-Streptomycin and Penicillin-Streptomycin. More preferably the medium may comprise 1% L-Alanyl-L-Glutamine, HEPES, 1% Penicillin-Streptomycin 10% heat-inactivated foetal bovine serum. Subsequently the IL-21 protein or IL-21 variant to be tested as described herein above or below may be added in a suitable amount. The final concentration in the mixture may preferably be between about 0.1 and 200 ng/ml, more preferably between about 0.5 and 100 ng/ml. In a further embodiment, additionally, IL-2 or IL-4 as described herein above or below, may be added in a suitable amount. The final concentration of each of these compounds in the mixture may preferably be between about 0.1 and 200 ng/ml, more preferably between about 0.5 and 100 ng/ml. Alternatively, also a combination of IL-2 and IL-4 may be added. The combination may be added in a suitable amount. The final concentration of combination in the mixture may preferably be between about 0.1 and 200 ng/ml, more preferably between about 0.5 and 100 ng/ml. Furthermore, a stimulator of CD40 molecules may be added in a suitable amount. Preferably, an anti-CD40 antibody, a CD40 ligand or C4BP may be added in a suitable amount. The final concentration of each of these compounds in the mixture may preferably be between about 0.5 to 4 μg/ml, more preferably between about 1 to 2 μg/ml. In a further step the mixed components may be incubated at a suitable incubation temperature known to the person skilled in the art, e.g. at 37° C. The incubation may be carried out in a beneficial atmosphere, preferably in the presence of 5% CO₂. The incubation may be carried out according to suitable rules known to the person skilled in the art. Preferably, the incubation may be carried out for a time period of 3 to 7 days. More preferably, the incubation may be carried out for a time period of 5 days.

Subsequently, the PBMC or selected B type lymphocytes may be washed with any suitable washing medium known to the skilled person, e.g. with IMDM, preferably with IMDM comprising 1% L-Alanyl-L-Glutamine, HEPES, 1% Penicillin-Streptomycin, and 10% heat-inactivated foetal bovine serum. The washing is preferably carried out on ice and may be repeated as often as suitable, e.g. twice.

For the determination of the IgG and IgA isotpye secretion of cultured PBMC or B type lymphocytes a suitable enzyme-linked immunosorbent spot forming assay (ELISPOT) as known to the skilled person may be used. The ELISPOT approach is typically carried out like a sandwich ELISA test, i.e. a capture antibody captures the protein of interest, which may subsequently be detected by a detection antibody.

Typically, a 96-well polyvinylidenefluoride-membrane (PVDF) filter plate may be pre-wet with 30% ethanol, rinsed three times with a sterile phosphate buffered saline (sPBS) and coated overnight at 4° C. with human immunoglobulin heavy chain specific polyclonal capture antibodies, diluted in sPBS at 5 to 15 μg/ml, preferably 10 μg/ml. Subsequently, the filter plate may be rinsed three times with sPBS and blocked several hours, preferably for 3 hrs, with sPBS containing 1% bovine serum albumin. Afterwards, the cultured PBMC, or B type lymphocytes may be plated at between 1×10³ and 1×10⁵ cells per well in 100 μl of IMDM with 1% L-Alanyl-L-Glutamine, HEPES, 1% Penicillin-Streptomycin, and 10% heat-inactivated foetal bovine serum. The ELISPOT filter plate may then be incubated at a suitable temperature, typically at 37° C. for a suitable time period, e.g. 14 to 28 hrs, preferably 20 hrs, in the presence of 5% CO₂. Thereafter, the ELISPOT filter plate may be washed six times using sPBS containing 0.01% Tween20 (PBS-Tween). Subsequently, human immunoglobulin heavy chains of the IgG and IgA isotype may be detected with any suitable means known to the person skilled in the art, e.g. with specific immunoglobulin heavy chain IgG and IgA isotype detection antibodies. Preferably polyclonal antibodies against the immunoglobulin heavy chain of IgG and/or IgA may be used. More preferably an anti-IgG/IgA antibody available from DAKO Cytomation and an unconjugated polyclonal goat anti-human IgG/IgA antibodies available from SouthernBiotech, Birmingham, Ala., USA may be used. In order to detect IgG and IgA at the same time different fluorescent labels may be used in conjunction with a corresponding reading system, e.g. the “iSpot”-Reader system provided by AID Diagnostika GmbH. Alternatively, specific antibodies for IgG and/or IgA subclasses as described herein above may be used. Preferred are IgG1 and IgG3 specific monoclonal antibodies, e.g. IgG1-mouse Anti-Human IgG1 (γ1 chain specific, clone 4E3), IgG3-mouse Anti-Human IgG3 (γ3 chain specific, clone HP6050) available from SouthernBiotech Inc.

Typically, the capture antibodies may be diluted in sPBS and be added at a final concentration of 1 to 3 μg/ml, preferably 2 μg/ml; the detection antibodies may be diluted in sPBS containing 0.5% bovine serum albumin and be added at a final concentration of 1 to 3 μg/ml, preferably 2 μg/ml.

The detection antibodies may preferably be biotinylated for streptavidin-coupling or directly conjugated with any enzyme suitable for colouring reactions, e.g. horseradish peroxidase (HRP) or alkaline phosphatase (AP).

After an overnight incubation at 4° C., the ELISPOT filter plate may be washed for additional six times with PBS-Tween. The binding reaction may be tested with any suitable colouring reaction known to the person skilled in the art. For example, the enzyme colouring reaction may be carried out by using 5-bromo-4-chloro-3-indolyl-phosphate and nitroblue tetrazolium (BCIP/NBT) as phosphatase substrate. The development of coloured spots, corresponding to a single cell that has secreted the immunoglobulin molecule of interest, can be directly monitored on the PVDF membrane for a period of time between 1 and 60 minutes. Microscopic analysis of each well of the ELISPOT filter plate and subsequent enumeration of cell counts and immunoglobulin amount can be performed by any suitable method known to the person skilled in the art, e.g. by using an automated high-resolution plate reader like the AID EliSpot 04 HR Reader system, and by using appropriate reader software. The amount of secreted immunoglobulin, i.e. the amount of secreted immunoglobulin of the isotype IgG and/or IgA, may be measured by any suitable method or based on any suitable unit known to the skilled person, preferably by using a virtual unit that is equivalent to the surface in (0.01 mm)² multiplied by the intensity of a particular spot.

A molecule, in particular an IL-21 protein or an IL-21 variant is regarded to be capable of increasing the secretion of IgG and/or IgA antibodies in B cells if the amount of secreted immunoglobulin as measured by the above described procedure, either with the addition of IL-2 and/or IL-4, or preferably without the addition of IL-2 or IL-4 during the procedure, is raised by at least 0.2%, preferably by at least 0.5%, 0.75%, 1%, 2%, 3%, 4%, 5%, 6%, 7%, 8%, 9%, 10% or higher, more preferably by at least 5% in comparison to the amount of secreted immunoglobulin as measured by the above described procedure when using the IL-21 protein as defined in SEQ ID NO:1 under otherwise identical conditions.

Preferably, a molecule, in particular an IL-21 protein or an IL-21 variant is regarded to be capable of increasing the secretion of IgG antibodies in B cells if the amount of secreted IgG isotype immunoglobulin as measured by the above described procedure, either with the addition of IL-2 and/or IL-4, or preferably without the addition of IL-2 or IL-4 during the procedure, is raised by at least 0.2%, preferably by at least 0.5%, 0.75%, 1%, 2%, 3%, 4%, 5%, 6%, 7%, 8%, 9%, 10% or higher, more preferably by at least 5% in comparison to the amount of secreted IgG isotype immunoglobulin as measured by the above described procedure when using the IL-21 protein as defined in SEQ ID NO:1 under otherwise identical conditions; and a molecule, in particular an IL-21 protein or an IL-21 variant is regarded to be capable of increasing the secretion of IgA antibodies in B cells if the amount of secreted IgA isotype immunoglobulin as measured by the above described procedure, either with the addition of IL-2 and/or IL-4, or preferably without the addition of IL-2 or IL-4 during the procedure, is raised by at least 0.2%, preferably by at least 0.5%, 0.75%, 1%, 2%, 3%, 4%, 5%, 6%, 7%, 8%, 9%, 10% or more, more preferably by at least 5% in comparison to the amount of secreted IgA isotype immunoglobulin as measured by the above described procedure when using the IL-21 protein as defined in SEQ ID NO: 1 under otherwise identical conditions.

A molecule, in particular an IL-21 protein or an IL-21 variant may also be regarded to be capable of increasing the secretion of IgG and/or IgA antibodies in B cells if the amount of secreted immunoglobulin as measured by the above described procedure, wherein IL-2 or IL-4 are not added during the procedure, is raised by at least a factor 5, preferably by at least a factor, 10, 12, 14, 16, 18, 20, 22, 24, 26, 28, 30, 32, 34, 36, 38, 40 or higher, more preferably by at least a factor 30 in comparison to the amount of secreted immunoglobulin as measured by the above described procedure when using unstimulated B type lymphocytes under otherwise identical conditions.

Alternatively, a molecule, in particular an IL-21 protein or an IL-21 variant may also be regarded to be capable of increasing the secretion of IgG and/or IgA antibodies in B cells if the amount of secreted immunoglobulin as measured by the above described procedure, wherein IL-2 and/or IL-4 are added during the procedure, is raised by at least a factor 10, preferably by at least a factor 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72 74, 76, 78, 80 or higher, more preferably by at least a factor 60 in comparison to the amount of secreted immunoglobulin as measured by the above described procedure when using unstimulated B type lymphocytes under otherwise identical conditions.

Preferably, a molecule, in particular an IL-21 protein or an IL-21 variant may be regarded to be capable of increasing the secretion of IgG antibodies in B cells if the amount of secreted IgG isotype immunoglobulin as measured by the above described procedure, wherein IL-2 or IL-4 are not added during the procedure, is raised by at least a factor 10, preferably by at least a factor 12, 14, 16, 18, 20, 22, 24, 26, 28, 30, 32, 34, 36, 38, 40 or higher, more preferably by at least a factor 30 in comparison to the amount of secreted IgG isotype immunoglobulin as measured by the above described procedure when using unstimulated B type lymphocytes under otherwise identical conditions; and a molecule, in particular an IL-21 protein or an IL-21 variant is regarded to be capable of increasing the secretion of IgA antibodies in B cells if the amount of secreted IgA isotype immunoglobulin as measured by the above described procedure, wherein IL-2 or IL-4 are not added during the procedure, is raised by at least a factor 5, preferably by at least a factor, 10, 12, 14, 16, 18, 20, 22, 24, 26, 28, 30, 32, or higher, more preferably by at least a factor 24 in comparison to the amount of secreted IgA isotype immunoglobulin as measured by the above described procedure when using unstimulated B type lymphocytes under otherwise identical conditions.

Alternatively, a molecule, in particular an IL-21 protein or an IL-21 variant may be regarded to be capable of increasing the secretion of IgG antibodies in B cells if the amount of secreted IgG isotype immunoglobulin as measured by the above described procedure, wherein IL-2 or IL-4 are added during the procedure, is raised by at least a factor 20, preferably by at least a factor 22, 24, 26, 28, 30, 32, 34, 36, 38, 40, 42, 44, 46, 48, 50, 52, 54, 56, 58, 60, 62, 64, 66, 68, 70, 72 74, 76, 78, 80 or higher, more preferably by at least a factor 60 in comparison to the amount of secreted IgG isotype immunoglobulin as measured by the above described procedure when using unstimulated B type lymphocytes under otherwise identical conditions; and a molecule, in particular an IL-21 protein or an IL-21 variant is regarded to be capable of increasing the secretion of IgA antibodies in B cells if the amount of secreted IgA isotype immunoglobulin as measured by the above described procedure, wherein IL-2 or IL-4 are added during the procedure, is raised by at least a factor 10, preferably by at least a factor 12, 14, 16, 18, 20, 22, 24, 26, 28, 30, 32, 34, 36, 38, 40, 42, 44, 46, 48, 50, 52, 54, 56, 58, 60, 62, 64, or higher, more preferably by at least a factor 40 in comparison to the amount of secreted IgA isotype immunoglobulin as measured by the above described procedure when using unstimulated B type lymphocytes under otherwise identical conditions.

Alternatively the capability of a protein, in particular of an IL-21 protein or IL-21 variant to increase the secretion of IgG and/or IgA antibodies in B cells may be tested via the verification and determination of induction of immunoglobulin class switch recombination in human B cells, preferably ex vivo. The term “immunoglobulin class switch recombination (CSR)” refers to an irreversible DNA-editing biological mechanism that changes the heavy chain of an immunoglobulin molecule from one class to another, e.g. from the IgM isotype to the IgG isotype. Typically, CSR occurs after adequate stimulation of mature B cells by chromosomal deletions of undesired heavy chain loci and rejoining of a single remaining heavy chain locus that represents the switch result. The process is normally linked to the expression of the enzyme activation-induced cytidine deaminase (AID). The term “AID expression” means that the enzyme AID is induced specifically in B cells to initiate CSR upon adequate stimulation of B cells. AID typically removes the amino group from the cytidine base on single stranded DNA during CSR and is therefore generally considered as master regulator of the CSR and marker for initiation of CSR processes. Typically, early steps during the CSR are characterised by the production of germline transcripts and the activity of germline transcription. The term “germline transcript” refers to a transcript being a part of the immunoglobulin heavy chain loci itself and typically consisting of a certain switch region and corresponding heavy chain locus, e.g. Sγ1 and Cγ1 or Sα and Cα. Germline transcription may correspondingly be quantified and serve as a marker to determine the direction and extent of the CSR. Preferably, switch circle transcripts may be detected, since these transcripts specifically reflect CSR events. The term “circle transcript” means that heavy chain loci which are excised during the DNA-editing steps of CSR form steric circles before their degradation. Circle transcripts therefore provide a reliable parameter for the detection of ongoing CSR.

In principle, the determination of induction of immunoglobulin class switch recombination in human B cells may be carried out by any suitable methods known to the person skilled in the art. Preferably, the determination of induction of immunoglobulin class switch recombination in human B cells may be carried out by using a method based on Klapper W, et al. (2006). J Pathol. 209 (2), 250-7.

A typical method to be employed in the context of the present invention may comprise, as a first step, the obtainment of human peripheral blood mononuclear cells (PBMC) from peripheral venous blood by Ficoll separation, preferably according to Kreher C R, et al. (2003) J Immunol Methods, 278 (1-2), 79-93. The cells may be obtained for the purpose of the present procedure only from one patient type, e.g. a healthy individual, or a patient with an immunological disorder like CVID, preferably only from one individual patient. More preferably, the cells may be obtained only from a healthy patient type. The term “healthy” means that the patient is not afflicted with an immunological disorder, in particular not with CVID or IgAD. It is further preferred that the cells obtained from a healthy patient spontaneously produce no or only marginal amounts of IgG or IgA if unstimulated. Such a behavior may be tested by methods known to the person skilled in the art, e.g. based on assays described herein above or below. The term “marginal amounts” means that in an ELISPOT assay as described herein a limit of overall 200 units equivalent to the surface in (0.01 mm)² multiplied by the intensity of the counted spots may not be surpassed. Alternatively, commercially available cells may be used, which have been normalized with regard to the production of IgA and/or IgG. Subsequently, CD19 positive B cells and CD138 positive plasma cells may be isolated from PBMC by any suitable method, e.g. by nanoparticle-based immunomagnetic cell selection, preferably with selection kits (e.g. commercially available kits of Miltenyi Biotec Inc., STEMCELL Technologies Inc. etc.). In a next step, the PBMC or the selected B type lymphocytes may be cultured in any suitable culture medium as known to the person skilled in the art, e.g. in Iscove's Modified Dulbecco's medium (IMDM). For the culturing an amount of 1×10⁵ to 2×10⁶ cells (PBMC or selected B type lymphocytes) may be used. The amount of cells may be determined by any suitable means known to the person skilled in the art, e.g. by cell counting devices or machines, microscopy determination etc.

Preferably, the medium may comprise L-Alanyl-L-Glutamine, HEPES, Penicillin-Streptomycin and Penicillin-Streptomycin. More preferably the medium may comprise 1% L-Alanyl-L-Glutamine, HEPES, 1% Penicillin-Streptomycin 10% heat-inactivated foetal bovine serum. Subsequently the IL-21 protein or IL-21 variant to be tested as described herein above or below may be added in a suitable amount. The final concentration in the mixture may preferably be between about 0.1 and 200 ng/ml, more preferably between about 0.5 and 100 ng/ml. Additionally, IL-2 or IL-4 as described herein above or below, may be added in a suitable amount. The final concentration of each of these compounds in the mixture may preferably be between about 0.1 and 200 ng/ml, more preferably between about 0.5 and 100 ng/ml. Alternatively, also a combination of IL-2 and IL-4 may be added. The combination may be added in a suitable amount. The final concentration of combination in the mixture may preferably be between about 0.1 and 200 ng/ml, more preferably between about 0.5 and 100 ng/ml. Furthermore, a stimulator of CD40 molecules may be added in a suitable amount. Preferably, an anti-CD40 antibody, a CD40 ligand or C4BP may be added in a suitable amount. The final concentration of each of these compounds in the mixture may preferably be between about 0.5 to 4 μg/ml, more preferably between about 1 to 2 μg/ml. In a further step the mixed components may be incubated at a suitable incubation temperature known to the person skilled in the art, e.g. at 37° C. The incubation may be carried out in a beneficial atmosphere, preferably in the presence of 5% CO₂. The incubation may be carried out according to suitable rules known to the person skilled in the art. Preferably, the incubation may be carried out for a time period of 2 to 5 days. More preferably, the incubation may be carried out for a time period of 4 days.

Subsequently, PBMC or selected B type lymphocytes are washed with any suitable buffer known to the person skilled in the art, e.g. sterile phosphate buffered saline (sPBS). Afterwards, the cells are subjected to RNA extraction, using for instance RNA extraction kits, preferably commercially available kits from QIAGEN, Roche Applied Science etc. The extraction may be carried out according to the manufacturers' instructions. The extracted RNA may then be reverse-transcribed into complementary DNA (cDNA), e.g. by using commercially available cDNA kits, preferably kits form QIAGEN, Roche Applied Science etc. Subsequently, the cDNA may be subjected to real time PCR analysis, preferably quantitative real time PCR. For this analysis any suitable primer sequence known to the person skilled in the art may be used. Preferably sequence specific primers for the detection of β-actinand/or AID expression as well as germline transcripts and/or circle transcripts may be employed.

More preferably the primer sequences

| (SEQ ID NO: 17) | |
|---|---|
| β-actin-1 | 5′CCTGGGCATGGAGTCCTGTGG3′ |
| and | |
| (SEQ ID NO: 18) | |
| β-actin-2 | 5′CTGTGTTGGCGTACAGGTCTT3′ |

may be used for the detection of the β-actin expression;
the primer sequences

| (SEQ ID NO: 19) | |
|---|---|
| AID-1 | 5′CACAAACTCTTCCATCAGGC3′ |
| and | |
| (SEQ ID NO: 20) | |
| AID-2 | 5′CATCCCCACCCATAACAATC3′ |

may be used for the detection of AID expression;
the primer sequences

| (SEQ ID NO: 21) | |
|---|---|
| Iγ-consensus | 5′CTCAGCCAGGACCAAGGACA3, |
| (SEQ ID NO: 22) | |
| Cγ-consensus | 5′ACCACGCTGCTGAGGGAGTA3′, |
| (SEQ ID NO: 23) | |
| Cμ-antisense-1 | 5′AATCTGCCGGGGACTGAAAC3′, |
| (SEQ ID NO: 24) | |
| Iα-consensus | 5′TGAGTGGACCTGCCATGA3′, |
| (SEQ ID NO: 25) | |
| Cα-consensus | 5′CTGGGATTCGTGTAGTGCTT3′, |
| and | |
| (SEQ ID NO: 26) | |
| Cμ-antisense-2 | 5′CGTCTGTGCCTGCATGACG3′ |

may be used for the detection of germline transcripts and/or circle transcripts.

Even more preferably, primer pairs with the sequences Iγ-consensus (SEQ ID NO: 21) and Cγ-consensus (SEQ ID NO: 22) may be used for the detection of IgG germline transcript expression; primer pairs with the sequences Iγ-consensus (SEQ ID NO: 21) and Cμ-antisense-1 (SEQ ID NO: 23) may be used for the detection of IgG switch circle transcript expression; primer pairs with the sequences Iα-consensus (SEQ ID NO: 24) and Cα-consensus (SEQ ID NO: 25) may be used for the detection of IgA germline transcript expression and primer pairs with the sequences Iα-consensus (SEQ ID NO: 24) and Cα-antisense-2 (SEQ ID NO: 26) may be used for the detection of IgA switch circle transcript expression.

Quantitative real time PCR may be carried out according to suitable and known procedural rules, preferably according to the manufacturers' instructions for PCR machines and/or real time PCR equipment and kits, e.g. instructions from Roche Applied Science Inc., Applied Biosystems Inc., etc.

Quantitative real time PCR results may be obtained and verified by suitable computer equipment and software as known to the person skilled in the art. An additional assessment of the quality of the results and an adjustment between different sample probes may be achieved by carrying out control reactions. Preferably, a control reaction involving the expression of p-actin for inter-sample normalisation may be used. The results may be presented in any suitable form, preferably as ratio of the calculated amount of candidate RNA in a given sample by the calculated amount of the control β-actin gene in the same sample.

A molecule, in particular an IL-21 protein or an IL-21 variant may be regarded to be capable of increasing the secretion of IgG or IgA antibodies in B cells if AID expression, IgG or IgA germline transcripts or circle transcripts can be detected according to the above described test.

Preferably, a molecule, in particular an IL-21 protein or an IL-21 variant may be regarded to be capable of increasing the secretion of IgG antibodies in B cells if the expression rate of AID measured as ratio of the calculated amount of candidate RNA in a given sample by the calculated amount of the control β-actin gene in the same sample is at least >6, preferably >7 and more preferably >8, if the expression rate of the IgG germline transcripts measured as ratio of the calculated amount of candidate RNA in a given sample by the calculated amount of the control β-actin gene in the same sample is at least >8, preferably >9 and more preferably >10; or if the expression rate of the IgG switch circle transcripts measured as ratio of the calculated amount of candidate RNA in a given sample by the calculated amount of the control β-actin gene in the same sample is at least >3, preferably >4 and more preferably >5.

Preferably, a molecule, in particular an IL-21 protein or an IL-21 variant may be regarded to be capable of increasing the secretion of IgA antibodies in B cells if the expression rate of the IgA germline transcripts measured as ratio of the calculated amount of candidate RNA in a given sample by the calculated amount of the control β-actin gene in the same sample is at least >3, preferably >4 and more preferably >5; or if the expression rate of the IgA switch circle transcripts measured as ratio of the calculated amount of candidate RNA in a given sample by the calculated amount of the control β-actin gene in the same sample is at least >1, preferably >2 and more preferably >3.

The test for determining whether a molecule, in particular an IL-21 protein or an IL-21 variant is capable of increasing the secretion of IgG and/or IgA antibodies in B cells is preferably a test as described herein above and more preferably a test as described in the Examples.

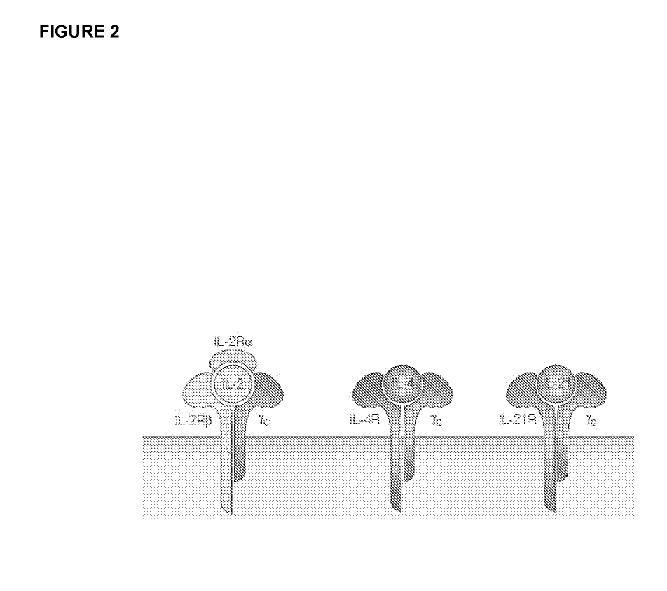

The term “capable of binding the IL-2 receptor complex” means that an IL-21 variant protein or IL-21 variant according to the invention can bind to the receptor complex of IL-2R and γc (see, for example, FIG. 2, which depicts an interaction between IL-2, IL-2Rα and IL-2Rβ and γc). The binding of the IL-21 variant to the IL-2 receptor complex may be tested by any suitable methods known to the skilled person, e.g. an assay as described herein, which is modified by the addition of equimolar amounts of recombinant human IL-2alpha receptor, e.g. an IL-2alpha receptor obtainable from R&D Systems, Minneapolis, Minn., USA. Preferred examples are recombinant Human IL-2Rα/Fc Chimera available from R&D Systems, Minneapolis, Minn., USA. Typically, in such an approach the effect of an IL-21 variant according to the present invention may linearly decrease with its capability of binding to the recombinant IL-2 receptor. As a control any suitable protein, preferably human wildtype IL-21, more preferably IL-21 as defined in SEQ ID NO: 1, “Chim-hIL-21/4”, IL-2, preferably IL-2 as defined in SEQ ID NO: 3 or an unrelated protein like BSA may be used.

A molecule, in particular an IL-21 protein or an IL-21 variant is regarded to be capable of binding the IL-2 receptor complex if the binding capability of the wildtype IL-21, preferably of IL-21 as defined in SEQ ID NO: 1, to the IL-2 receptor complex, is increased by at least 15%, preferably by at least 20%, 25%, 30%, 40%, 50%, 60%, 70%, 80%, 90%, 100%, 200%, 300%, 500%, 750%, 1000%, 2000%, 5000%, 7500%, 10,000% or more, or is increased by at least a factor 5, 7, 10, 12, 14, 16, 18, 20, 22, 24, 26, 28, 30, 32, 34, 36, 38, 40, 50, 100, 150, 200, 500, 1000, 5000, 10000, 100000, 500000, 10⁶ or higher, as measured by any suitable method, more preferably as measured by the above described procedure.

A molecule, in particular an IL-21 protein or an IL-21 variant may also be regarded to be capable of binding the IL-2 receptor complex if said IL-21 variant is able to bind to the IL-2 receptor complex with an affinity of at least about 0.2%, preferably of at least 0.5%, 0.75%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 33%, 35%, 37%, 40%, 43%, 45%, 47%, 50%, 53%, 55%, 57%, 60%, 63%, 65%, 67%, 70% or 75% or more of the affinity of the wildtype IL-2, preferably of IL-2 as defined in SEQ ID NO: 3, to the IL-2 receptor complex, as measured by any suitable method, more preferably as measured by the above described procedure.

A molecule, in particular an IL-21 protein or an IL-21 variant may also be regarded to be capable of binding the IL-2 receptor complex if said IL-21 variant is able to bind to the IL-2 receptor complex with an affinity, i.e. a dissociation constant (K_{d}) of at least about 1.0×10⁻⁶ M, 2.0×10⁻⁶ M, 3.0×10⁻⁶ M, 5.0×10⁻⁶ M, 7.5×10⁻⁶ M, 1.0×10⁻⁷ M, 2.0×10⁻⁷ M, 3.0×10⁻⁷ M, 4.0×10⁻⁷ M, 5.0×10⁻⁷ M, 6.0×10⁻⁷ M, 7.0×10⁻⁷ M, 8.0×10⁻⁷ M, 9.0×10⁻⁷ M, 1.0×10⁻⁸ M, 2.0×10⁻⁸ M, 3.0×10⁻⁸ M, 4.0×10⁻⁸ M, 5.0×10⁻⁸ M, 6.0×10⁻⁸ M, 7.0×10⁻⁸ M, 8.0×10⁻⁸ M, 9.0×10⁻⁸ M, 1.0×10⁻⁹ M, 2.0×10⁻⁹ M, 3.0×10⁻⁹ M, 4.0×10⁻⁹ M, 5.0×10⁻⁹ M, 6.0×10⁻⁹ M, 7.0×10⁻⁹ M, 8.0×10⁻⁹ M, 9.0×10⁻⁹ M, 1.0×10⁻¹⁰ M, 2.0×10⁻¹⁰ M, 3.0×10⁻¹⁰ M, 4.0×10⁻¹⁰ M, 5.0×10⁻¹⁰ M, 6.0×10⁻¹⁰ M, 7.0×10⁻¹⁰ M, 8.0×10⁻¹⁰ M, 9.0×10⁻¹⁰ M, 1.0×10⁻¹¹ M, 2.0×10⁻¹¹ M, 3.0×10⁻¹¹ M, 4.0×10⁻¹¹ M, 5.0×10⁻¹¹ M, 6.0×10⁻¹¹ M, 7.0×10⁻¹¹ M, 8.0×10⁻¹¹ M, or 9.0×10⁻¹¹ M. The affinity may be measured and calculated according to any suitable method known to the person skilled in the art, for example according to a modification of the Scatchard method described by Frankel et al., (1979), Mol. Immunol., 16: 101-106, or via a competition radioimmunoassay, or by ELISA testings.

The term “capable of binding the IL-4 receptor complex” means that an IL-21 variant protein or IL-21 variant according to the invention can bind to the receptor complex of IL-4R and γc (see, for example, FIG. 2, which depicts an interaction between IL-4, IL-4R and γc). The binding of the IL-21 variant to the IL-4 receptor complex may be tested by any suitable methods known to the skilled person, e.g. an assay as described herein, which is modified by the addition of equimolar amounts of recombinant human IL-4alpha receptor, e.g. an IL-4alpha receptor obtainable from R&D Systems, Minneapolis, Minn., USA. Preferred examples are recombinant Human IL-4 Rα/Fc Chimera available from R&D Systems, Minneapolis, Minn., USA. Typically, in such an approach the effect of an IL-21 variant according to the present invention may linearly decrease with its capability of binding to the recombinant IL-4 receptor. As a control any suitable protein, preferably human wildtype IL-21, more preferably IL-21 as defined in SEQ ID NO: 1, “Chim-hIL-21/4”, IL-4, preferably IL-4 as defined in SEQ ID NO: 2 or an unrelated protein like BSA may be used.

A molecule, in particular an IL-21 protein or an IL-21 variant is regarded to be capable of binding the IL-4 receptor complex if the binding capability of the wildtype IL-21, preferably of IL-21 as defined in SEQ ID NO: 1, to the IL-4 receptor complex, is increased by at least 15%, preferably by at least 20%, 25%, 30%, 40%, 50%, 60%, 70%, 80%, 90%, 100%, 200%, 300%, 500%, 750%, 1000%, 2000%, 5000%, 7500%, 10,000% or more, or is increased by at least a factor 5, 7, 10, 12, 14, 16, 18, 20, 22, 24, 26, 28, 30, 32, 34, 36, 38, 40, 50, 100, 150, 200, 500, 1000, 5000, 10000, 100000, 500000, 10⁶ or higher, as measured by any suitable method, more preferably as measured by the above described procedure.

A molecule, in particular an IL-21 protein or an IL-21 variant may also be regarded to be capable of binding the IL-4 receptor complex if said IL-21 variant is able to bind to the IL-4 receptor complex with an affinity of at least about 0.2%, preferably of at least 0.5%, 0.75%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 33%, 35%, 37%, 40%, 43%, 45%, 47%, 50%, 53%, 55%, 57%, 60%, 63%, 65%, 67%, 70% or 75% or more of the affinity of the wildtype IL-4, preferably of IL-4 as defined in SEQ ID NO: 2, to the IL-4 receptor complex, as measured by any suitable method, more preferably as measured by the above described procedure.

A molecule, in particular an IL-21 protein or an IL-21 variant may also be regarded to be capable of binding the IL-4 receptor complex if said IL-21 variant is able to bind to the IL-4 receptor complex with an affinity, i.e. a dissociation constant (K_{d}) of at least about 1.0×10⁻⁶ M, 2.0×10⁻⁶ M, 3.0×10⁻⁶ M, 5.0×10⁻⁶ M, 7.5×10⁻⁶ M, 1.0×10⁻⁷ M, 2.0×10⁻⁷ M, 3.0×10⁻⁷ M, 4.0×10⁷ M, 5.0×10⁻⁷ M, 6.0×10⁻⁷ M, 7.0×10⁻⁷ M, 8.0×10⁻⁷ M, 9.0×10⁻⁷ M, 1.0×10⁻⁸ M, 2.0×10⁻⁸ M, 3.0×10⁻⁸ M, 4.0×10⁻⁸ M, 5.0×10⁻⁸ M, 6.0×10⁻⁸ M, 7.0×10⁻⁸ M, 8.0×10⁻⁸ M, 9.0×10⁻⁸ M, 1.0×10⁻⁹ M, 2.0×10⁻⁹ M, 3.0×10⁻⁹M, 4.0×10⁻⁹ M, 5.0×10⁻⁹ M, 6.0×10⁻⁹ M, 7.0×10⁻⁹ M, 8.0×10⁻⁹ M, 9.0×10⁻⁹ M, 1.0×10⁻¹⁰ M, 2.0×10⁻¹⁰ M, 3.0×10⁻¹⁰ M, 4.0×10⁻¹⁰ M, 5.0×10⁻¹⁰ M, 6.0×10⁻¹⁰ M, 7.0×10⁻¹⁰ M, 8.0×10⁻¹⁰ M, 9.0×10⁻¹⁰ M, 1.0×10⁻¹¹ M, 2.0×10⁻¹¹ M, 3.0×10⁻¹¹ M, 4.0×10⁻¹¹ M, 5.0×10⁻¹¹ M, 6.0×10⁻¹¹ M, 7.0×10⁻¹¹ M, 8.0×10⁻¹¹ M, or 9.0×10⁻¹¹ M. The affinity may be measured and calculated according to any suitable method known to the person skilled in the art, for example according to a modification of the Scatchard method described by Frankel et al., (1979), Mol. Immunol., 16: 101-106, or via a competition radioimmunoassay, or by ELISA testings.

The term “capable of binding the IL-2 and the IL-4 receptor complex” means that an IL-21 variant protein or IL-21 variant according to the invention can bind to both, the receptor complex of IL-2R and γc and the receptor complex of IL-4R and γc. The binding of the IL-21 variant to the IL-2 and the IL-4 receptor complexs may be tested by any suitable methods known to the skilled person, e.g. an assay as described herein above. As a control any suitable protein, preferably human wildtype IL-21, more preferably IL-21 as defined in SEQ ID NO: 1, “Chim-hIL-21/4”, IL-4, preferably IL-4 as defined in SEQ ID NO: 2, IL-2, preferably IL-2 as defined in SEQ ID NO: 3, or an unrelated protein like BSA may be used.

A molecule, in particular an IL-21 protein or an IL-21 variant is regarded to be capable of binding the IL-2 and the IL-4 receptor complex if the averaged binding capability of the wildtype IL-21, preferably of IL-21 as defined in SEQ ID NO: 1, to the IL-2 and the IL-4 receptor complex, is increased by at least 15%, preferably by at least 20%, 25%, 30%, 40%, 50%, 60%, 70%, 80%, 90%, 100%, 200%, 300%, 500%, 750%, 1000%, 2000%, 5000%, 7500%, 10,000% or more, or is increased by at least a factor 5, 7, 10, 12, 14, 16, 18, 20, 22, 24, 26, 28, 30, 32, 34, 36, 38, 40, 50, 100, 150, 200, 500, 1000, 5000, 10000, 100000, 500000, 10⁶ or higher, as measured by any suitable method, more preferably as measured by the above described procedure.

A molecule, in particular an IL-21 protein or an IL-21 variant may also be regarded to be capable of binding the IL-2 and the IL-4 receptor complex if said IL-21 variant is able to bind to the IL-2 and the IL-4 receptor complex with an averaged affinity of at least about 0.2%, preferably of at least 0.5%, 0.75%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 33%, 35%, 37%, 40%, 43%, 45%, 47%, 50%, 53%, 55%, 57%, 60%, 63%, 65%, 67%, 70% or 75% or more of the affinity of the wildtype IL-2, preferably of IL-2 as defined in SEQ ID NO: 3, to the IL-2 receptor complex, or of IL-4, preferably of IL-4 as defined in SEQ ID NO: 2, to the IL-4 receptor complex, respectively, as measured by any suitable method, more preferably as measured by the above described procedure.

A molecule, in particular an IL-21 protein or an IL-21 variant may also be regarded to be capable of binding the IL-2 and the IL-4 receptor complex if said IL-21 variant is able to bind to the IL-2 and the IL-4 receptor complex with an averaged affinity, i.e. a dissociation constant (K_{d}) of at least about 1.0×10⁻⁶ M, 2.0×10⁻⁶ M, 3.0×10⁻⁶ M, 5.0×10⁻⁶ M, 7.5×10⁻⁶ M, 1.0×10⁻⁷ M, 2.0×10⁻⁷ M, 3.0×10⁻⁷ M, 4.0×10⁻⁷ M, 5.0×10⁻⁷ M, 6.0×10⁻⁷ M, 7.0×10⁻⁷ M, 8.0×10⁻⁷ M, 9.0×10⁻⁷ M, 1.0×10⁻⁸ M, 2.0×10⁻⁸ M, 3.0×10⁻⁸ M, 4.0×10⁻⁸ M, 5.0×10⁻⁸ M, 6.0×10⁻⁸ M, 7.0×10⁻⁸ M, 8.0×10⁻⁸ M, 9.0×10⁻⁸ M, 1.0×10⁻⁹ M, 2.0×10⁻⁹ M, 3.0×10⁻⁹ M, 4.0×10⁻⁹ M, 5.0×10⁻⁹ M, 6.0×10⁻⁹ M, 7.0×10⁻⁹ M, 8.0×10⁻⁹ M, 9.0×10⁻⁹ M, 1.0×10⁻¹⁰ M, 2.0×10⁻¹⁰ M, 3.0×10⁻¹⁰ M, 4.0×10⁻¹⁰⁻¹⁰ M, 5.0×10⁻¹⁰ M, 6.0×10⁻¹⁰ M, 7.0×10⁻¹⁰ M, 8.0×10⁻¹⁰ M, 9.0×10⁻¹⁰ M, 1.0×10⁻¹¹ M, 2.0×10⁻¹¹ M, 3.0×10⁻¹¹ M, 4.0×10⁻¹¹ M, 5.0×10⁻¹¹ M, 6.0×10⁻¹¹ M, 7.0×10⁻¹¹ M, 8.0×10⁻¹¹ M, or 9.0×10⁻¹¹ M.

In a further preferred embodiment an IL-21 protein or IL-21 variant according to the present invention may be capable of increasing the secretion of IgG and/or

IgA and be capable of binding the IL-2 receptor complex and/or the IL-4 receptor complex. In a further preferred embodiment an IL-21 protein or IL-21 variant according to the present invention may be capable of increasing the secretion of IgG and be capable of binding the IL-2 receptor complex. In a further preferred embodiment an IL-21 protein or IL-21 variant according to the present invention may be capable of increasing the secretion of IgG and be capable of binding the IL-4 receptor complex. In a further preferred embodiment the an IL-21 protein or IL-21 variant according to the present invention may be capable of increasing the secretion of IgA and be capable of binding the IL-2 receptor complex. In a further preferred embodiment the an IL-21 protein or IL-21 variant according to the present invention may be capable of increasing the secretion of IgA and be capable of binding the IL-4 receptor complex.

In a further embodiment of the present invention an IL-21 protein or IL-21 variant according to the present invention which is capable of binding the IL-2 receptor complex and/or the IL-4 receptor complex may additionally be capable of exerting an agonistic, antagonistic, partial agonistic and/or partial antagonistic effect on the IL-21 receptor complex, the IL-2 receptor complex and/or the IL-4 receptor complex. In a preferred embodiment, said IL-21 variant may be capable of exerting a partial agonistic effect on the IL-21 receptor complex and the IL-2 receptor complex, or on the IL-21 receptor complex and the IL-4 receptor complex. The term “partial agonistic” or “partial antagonistic” as used herein refers to an agonistic or antagonistic effect of the IL-21 variant which is below the the effect of a full agonist, e.g. the cognate or wildtype ligand for the IL-21, IL-2 or IL-4 receptor complex, respectively, or below the effect of a full antagonist of said receptor complexes. The term “below” as used herein may preferably refer to an effect of at least 90%, 85%, 75%, 60%, 50%, 40%, 20%, 10%, 5%, 1% or 0.5% of the full agonistic or antagonistic effect as mentioned above.

For the purpose of the present invention an “IL-21 variant” may comprise a signal sequence or may not comprise a signal sequence. If the IL-21 variant comprises a signal sequence it may typically comprise the IL-21 signal sequence. The term “IL-21 signal sequence” relates to the 29 amino acids signal sequence shown in the amino acids of positions 1 to 29 of SEQ ID NO: 4. Alternatively, an “IL-21 variant” may also comprise any other suitable signal sequence known to the person skilled in the art, e.g. the signal sequence of IL-2 or IL-4. The term “signal sequence of IL-2” relates to the 20 amino acids signal sequence shown in the amino acids of positions 1 to 20 of SEQ ID NO: 5. The term “signal sequence of IL-4” relates to the 24 amino acids signal sequence shown in the amino acids of positions 1 to 24 of SEQ ID NO: 6.

The term “derived” as use in the context of IL-21 variants means that the human IL-21 is modified by the substitution of one or more amino acids and/or one or more amino acid stretches by one or more amino acids and/or one or more amino acid stretches of other interleukins, preferably of IL-2 and/or IL-4. The amino acids to be substituted may be located at any position throughout the IL-21 molecule, e.g. at the N-terminus, at the C-terminus, or in the central portion. The amino acids to be substituted may comprise amino acids being positioned in any typical secondary or 3-dimensional protein structure like a helical portion, a beta-sheet, a beta-bridge, a bonded turn or a bend. The term “secondary protein structure” preferably relates to the 3-dimensional protein structure as defined in the Dictionary of Protein Secondary Structure (DSSP; Kabsch W., et al. (1983). Biopolymers 22 (12), 2577-2637). According to the DSSP method the protein secondary structure is typically described with single letter codes. The secondary structure may be assigned based on hydrogen bonding patterns. Typically, there are eight types of secondary structure which the DSSP method describes: G=3-turn helix (3₁₀ helix) with a minimum length of 3 residues. H=4-turn helix (α helix) with a minimum length of 4 residues. I=5-turn helix (π helix) with a minimum length of 5 residues. T=hydrogen bonded turn (3, 4 or 5 turn). E=extended strand in parallel and/or anti-parallel β-sheet conformation with a minimum length of 2 residues. B=residue in isolated β-bridge (single pair β-sheet hydrogen bond formation) and S=bend (the only non-hydrogen-bond based assignment). Amino acid residues which are not in any of the above conformations are assigned as the eighth type ‘Coil’: typically codified as C (coil). The helices (G, H and I) and sheet conformations are normally required to have a reasonable length. Accordingly 2 adjacent residues in the primary structure must form the same hydrogen bonding pattern. If the helix or sheet hydrogen bonding pattern is too short they are designated as T or B, respectively.

The secondary structure of a protein, e.g. of IL-21, of an IL-21 variant, of IL-2 or IL-4 or of any variant of IL-2 or IL-4 etc. may be predicted by suitable methods known to the person skilled in the art. Typically methods of secondary-structure prediction may be used which are based on the helix- or sheet-forming propensities of individual amino acids, optionally coupled with rules for estimating the free energy of forming secondary structure elements. Furthermore, multiple sequence alignments may be exploited, thus using the full distribution of amino acids that occur at a position and in its vicinity, typically about 7 residues on either side throughout evolution. A further typical prediction approach is the examination of the average hydrophobicity or residue solvent accessibility at a certain position and at nearby positions. By combining alignment data and hydrophobicity or residue solvent accessibility data the accuracy of the prediction may be raised. Typically mathematical methods including neural networks, hidden Markov models and support vector machines may be used for the prediction of secondary structures of the protein. The amino acids to be substituted in IL-21 may comprise any number of amino acids as long as the modified polypeptide is capable of increasing the secretion of IgG and/or IgA antibodies in B cells. Preferably, the number of substituted amino acids is between about 0.5% and about 50% of all amino acids of IL-21, more preferably between about 5% and 40% of all amino acids of IL-21 and even more preferably between about 10% and 30% of all amino acids of IL-21. In a further embodiment, the number of substituted amino acids is between about 1% and about 90% of one type of secondary structure as defined herein above, preferably between about 1% and about 60% of helical structures, more preferably between about 1% and about 60% of H structures or α helices.

The amino acids to be substituted in IL-21 may be present in one or more stretches of amino acids of IL-21. Preferably, the substitution may be carried out in 1, 2, 4, 5, 6, 7, 8, 9, 10, 11, 12, 13, 14 or 15 stretches. The present invention also encompasses the substitution of more stretches of amino acids of IL-21, up to about 100. The term “stretch of amino acids” refers to a number of at least two adjacent amino acids. A stretch of amino acids may also encompass the entire number of adjacent amino acids of a protein, e.g. of IL-21. Preferably, a stretch of amino acids to be substituted is between about 3 and 60 adjacent amino acids, more preferably between about 5 and 40 adjacent amino acids and even more preferably between about 10 and 30 adjacent amino acids in length.

Is more than one stretch of amino acids to be substituted in IL-21, the stretches may be either in close proximity, e.g. only detached by one, two or three amino acids, or located at the opposite extremities of the IL-21 primary structure. Alternatively, the stretches of may be located anywhere throughout the IL-21 primary structure. The term “primary structure” as denoted herein above refers to the exact specification of a protein or polypeptide via its atomic composition and the chemical bonds connecting those atoms.

Are the stretches to be substituted located anywhere throughout the IL-21 primary structure, the stretches may or may not be located in close proximity in the secondary and/or tertiary structure of IL-21. The term “tertiary structure” as denoted herein above refers to the three-dimensional structure of a protein or polypeptide, as defined by its atomic coordinates. Typically, the tertiary structure of a protein, e.g. of IL-21, may be obtained via crystallographic or NMR spectroscopic analyses. Alternatively, in further embodiments of the present invention some stretches of amino acids to be substituted may be grouped at different 3-dimensional positions or regions, e.g. there may be one or more clusters of stretches of amino acids to be substituted in one or more 3-dimensional regions or positions of a protein.

In a further preferred embodiment of the present invention, the substituted amino acids or stretches of amino acids reside in portions of IL-21 which are responsible for the binding of IL-21 to its cognate receptor, preferably to the receptor complex of IL-21R and γc (see, for example, FIG. 2, which depicts an interaction between IL-21 and IL-21R and γc). The term “portions of IL-21 which are responsible for the binding of IL-21 to its cognate receptor” relates to 3-dimensional sections of human IL-21 which were implicated by NMR spectroscopy studies to be involved in the IL-21 receptor binding (Bondensgaard K. et al. (2007) J. Biol. Chem. 282 (32), 23326-36). Examples of such portions of IL-21 being responsible for the binding of IL-21 to its cognate receptor are helical or interhelical sections of IL-21 which provide protrusions in NMR spectroscopy tests or assays. Preferably, the amino acids or stretches of amino acids to be substituted may reside in one or more spatially cooperative helical regions of IL-21. The term “spatially cooperative helical regions of IL-21” relates to portions of IL-21 which are derivable or predictable from IL-21 structural data as being especially conjoined in a 3-dimensional structure of the protein. The amino acids or stretches of amino acids to be substituted may comprise the entire spatially cooperative helical regions of IL-21 or between about 0.5% and 95% of the spatially cooperative helical regions of IL-21, preferably about 20 to 60% of the spatially cooperative helical regions of IL-21. Methods to define the spatially cooperative helical regions of IL-21 would be known to the person skilled in the art or can be derived from any suitable structural biology textbook. Specific details for the definition of the spatially cooperative helical regions of IL-21 may be derived from Bondensgaard K. et al. (2007) J. Biol. Chem. 282 (32), 23326-36.

In a further embodiment of the present invention the amino acids or stretches of amino acids to be substituted may reside in helical regions A, B, C or D as defined in Bondensgaard K. et al. (2007) J. Biol. Chem. 282 (32), 23326-36. Alternatively or additionally, the amino acids or stretches of amino acids to be substituted may reside in interhelical regions or turns between regions A, B, C or D as defined in Bondensgaard K. et al. (2007) J. Biol. Chem. 282 (32), 23326-36. More preferably, the amino acids or stretches of amino acids to be substituted may reside in helix C and in the interhelical loop between helix C and D.

In a particularly preferred embodiment of the present invention amino acids or stretches of amino acids of IL-21 may be substituted in a way that at least one of the helices involved in the binding to the IL-21 receptor is not affected by modifications, preferably not by structural changes. More preferably, amino acids or stretches of amino acids of IL-21 may be substituted in a way that at least helix A or helix C, or helix A and helix C of IL-21 may not be affected by modifications, preferably not by structural changes. Even more preferably, such substitutions of amino acids or stretches of amino acids of IL-21 may be performed such that a modified IL-21 protein or variant is capable of binding to the IL-21 receptor complex as defined herein above. The binding of the IL-21 variant to the IL-21 receptor complex may be tested by any suitable methods known to the skilled person. Preferably the binding of the IL-21 variant to the IL-21 receptor complex may be tested in an assay as described herein above, which is modified by the addition of equimolar amounts of recombinant human IL-21 receptor, e.g. an IL-21 receptor obtainable from R&D Systems, Minneapolis, Minn., USA. A preferred example is recombinant Human Interleukin 21 Receptor/Fc Chimera available from R&D Systems, Minneapolis, Minn., USA. Typically, in such an approach the effect of an IL-21 variant according to the present invention may linearly decrease with its capability of binding to the recombinant IL-21 receptor. As a control any suitable protein, preferably human wildtype IL-21, more preferably IL-21 as defined in SEQ ID NO: 1 may be used. Corresponding modifications may be derived from the structural data provided in Bondensgaard K. et al. (2007) J. Biol. Chem. 282 (32), 23326-36.

In a further, particularly preferred embodiment of the present invention amino acids or stretches of amino acids of IL-21 may be substituted in a way that at least one, preferably two of the helices involved in the binding to the IL-21 receptor is affected by modifications, preferably by structural changes. More preferably, amino acids or stretches of amino acids of IL-21 may be substituted in a way that at least helix A or helix C, or helix A and helix C of IL-21 may be affected by modifications, preferably by structural changes. Even more preferably, such substitutions of amino acids or stretches of amino acids of IL-21 may be performed such that a modified IL-21 protein or variant is capable of binding to the IL-21 receptor complex as defined herein above. The binding of the IL-21 variant to the IL-21 receptor complex may be tested by any suitable methods known to the skilled person. Preferably the binding of the IL-21 variant to the IL-21 receptor complex may be tested in an assay as described herein above, which is modified by the addition of equimolar amounts of recombinant human IL-21 receptor, e.g. a IL-21 receptor obtainable from R&D Systems, Minneapolis, Minn., USA. A preferred example is recombinant Human Interleukin 21 Receptor/Fc Chimera available from R&D Systems, Minneapolis, Minn., USA. Typically, in such an approach the effect of an IL-21 variant according to the present invention may linearly decrease with its capability of binding to the recombinant IL-21 receptor. As a control any suitable protein, preferably human wildtype IL-21, more preferably IL-21 as defined in SEQ ID NO: 1 may be used.

In a further embodiment such substitutions of amino acids or stretches of amino acids of IL-21 may be performed such that a modified IL-21 protein or variant is additionally capable of binding to the IL-2 or IL-4 receptor complex as defined herein below, alternatively capable of binding to the IL-2 and IL-4 receptor complex as defined herein below. The binding of the IL-21 variant to the IL-21, IL-2 and/or IL-4 receptor complex may be tested by any suitable methods known to the skilled person. Preferably, the binding of the IL-21 variant to the IL-21, the IL-2 and/or IL-4 receptor complex may be tested in an assay as described herein above, which is modified by the addition of equimolar amounts of recombinant human IL-21 receptor, e.g. a IL-21 receptor obtainable from R&D Systems, Minneapolis, Minn., USA, of recombinant human IL-2alpha receptor, e.g. a IL-2alpha receptor obtainable from R&D Systems, Minneapolis, Minn., USA, or of recombinant human IL-4alpha receptor, e.g. a IL-4alpha receptor available from R&D Systems, Minneapolis, Minn., USA. Preferred examples are recombinant Human Interleukin 21 Receptor/Fc Chimera, Recombinant Human IL-2 Rα/Fc Chimera and Recombinant Human IL-4 Rα/Fc Chimera, all available from R&D Systems, Minneapolis, Minn., USA. Typically, in such an approach the effect of an IL-21 variant according to the present invention may linearly decrease with its capability of binding to the recombinant IL-21, IL-2 or IL-4 receptor. As a control any suitable protein, preferably human wildtype IL-21, more preferably IL-21 as defined in SEQ ID NO: 1 may be used. Corresponding modifications may be derived from the structural data provided in Bondensgaard K. et al. (2007) J. Biol. Chem. 282 (32), 23326-36; Hage T et al. (1999) Cell, 97 (2), 271-81; Zhang J L et al. (2002) J Mol Biol. 315 (3), 399-407; Stauber D J et al. (2006) PNAS 103 (8):2788-93; and Rickert M et al. (2005) Science, 308 (5727), 1477-80.

The term “substituted” or “in substitution of as denoted herein above means that a certain number of amino acids of IL-21 is replaced by the same number of amino acids derived from IL-2 or IL-4. The term also encompasses the replacement of a certain number of amino acids of IL-21 by a greater or smaller number of amino acids of IL-2 or IL-4. The stretches of amino acids integrated into the IL-21 protein may increase the size of the protein as defined in SEQ ID NO: 1 by at most 35%, preferably not more than 25% and even more preferably not more than 20%, 18%, 16%, 14%, 12%, 10%, 8%, 6%, 4%, 3%, 2% or 1%. The stretches of amino acids integrated into the IL-21 protein may alternatively also decrease the size of the protein as defined in SEQ ID NO: 1 by at most 35%, preferably not more than 25% and even more preferably not more than 20%, 18%, 16%, 14%, 12%, 10%, 8%, 6%, 4%, 3%, 2% or 1%.

The term “stretches of amino acids of IL-4 in substitution of amino acids of IL-21” refers to one or more amino acids of IL-4, preferably to a number of at least two adjacent amino acids of IL-4, which are to be transferred from IL-4 to IL-21. A stretch of amino acids may also encompass the entire number of adjacent amino acids of IL-4. Preferably, a stretch of amino acids of IL-4 to be substituted for amino acids of IL-21 is between about 3 and 60 adjacent amino acids, more preferably between about 5 and 40 adjacent amino acids and even more preferably between about 10 and 30 adjacent amino acids in length. The amino acids of IL-4 to be transferred to IL-21 or substituted for IL-21 amino acids may be located at any position throughout the IL-4 molecule, e.g. at the N-terminus, at the C-terminus, or in the central portion. The amino acids of IL-4 to be transferred may comprise amino acids being positioned in any typical secondary or 3-dimensional protein structure like a helical portion, a beta-sheet, a beta-bridge, a bonded turn or a bend.

The number of IL-4 amino acids to be transferred to IL-21 may preferably be between about 0.5% and about 50% of all amino acids of IL-4, more preferably between about 5% and 40% of all amino acids of IL-4 and even more preferably between about 10% and 30% of all amino acids of IL-4. In a further embodiment, the number of substituted amino acids may be between about 1% and about 90% of one type of secondary structure as defined herein above of IL-4, preferably between about 1% and about 60% of helical structures of IL-4, more preferably between about 1% and about 60% of H structures or a helices of IL-4.

The amino acids to be transferred from IL-4 to IL-21 may be present in one or more stretches of amino acids of IL-4. Preferably, a transfer or substitution may be carried out with 1, 2, 4, 5, 6, 7, 8, 9, 10, 11, 12, 13, 14 or 15 stretches of amino acids of IL-4. The present invention also encompasses the substitution of more stretches of amino acids of IL-4, up to about 100.

Is more than one stretch of amino acids to be transferred from IL-4, the stretches may be either derived from positions in close proximity, e.g. only detached by one, two or three amino acids, or be derived from positions located at the opposite extremities of the IL-4 primary structure. Alternatively, the stretches of may be located anywhere throughout the IL-4 primary structure.

Are the stretches to be transferred to IL-21 located anywhere throughout the IL-4 primary structure, the stretches may or may not be located in close proximity in the secondary and/or tertiary structure of IL-4. Typically, the tertiary structure of IL-4, may be obtained via by crystallographic or NMR spectroscopic analyses. Alternatively, in further embodiments of the present invention some stretches of amino acids to be transferred from IL-4 to IL-21 may be grouped at different 3-dimensional positions or regions of IL-4, e.g. there may be one or more clusters of stretches of amino acids to be transferred in one or more 3-dimensional regions or positions of a protein.

In a further preferred embodiment of the present invention, the transferred amino acids or stretches of amino acids of IL-4 reside in portions of IL-4 which are responsible for the binding of IL-4 to its cognate receptor, preferably to the receptor complex of IL-4R and γc (see, for example, FIG. 2, which depicts an interaction between IL-4 and IL-4R and γc). The term “portions of IL-4 which are responsible for the binding of IL-4 to its cognate receptor” relates to 3-dimensional sections of human IL-4 which were implicated by crystallographic studies to be involved in the IL-4 receptor binding (Hage T et al. (1999) Cell, 97 (2), 271-81; and Zhang J L et al. (2002) J Mol Biol. 315 (3), 399-407). Examples of such portions of IL-4 being responsible for the binding of IL-4 to its cognate receptor are helical or interhelical sections of IL-21 which provide protrusions in crystallographic tests or assays. Preferably, the amino acids or stretches of amino acids to be substituted may reside in one or more spatially cooperative helical regions of IL-4. The term “spatially cooperative helical regions of IL-4” relates to portions of IL-4 which are derivable or predictable from IL-4 structural data as being especially conjoined in a 3-dimensional structure of the protein. The amino acids or stretches of amino acids to be transferred may comprise the entire spatially cooperative helical regions of IL-4 or between about 0.5% and 95% of the spatially cooperative helical regions of IL-4, preferably about 20 to 60% of the spatially cooperative helical regions of IL-4. Methods to define the spatially cooperative helical regions of IL-4 would be known to the person skilled in the art or can be derived from any suitable structural biology textbook. Specific details for the definition of the spatially cooperative helical regions of IL-4 may be derived from Hage T et al. (1999) Cell, 97 (2), 271-81; and Zhang J L et al. (2002) J Mol Biol. 315 (3), 399-407.

In a preferred embodiment the amino acids or stretches of amino acids of IL-21 as defined herein above may be substituted by amino acids or stretches of amino acids IL-4 as defined in SEQ ID NO: 2.

The term “stretches of amino acids of IL-2 in substitution of amino acids of IL-21” refers to one or more amino acids of IL-2, preferably to a number of at least two adjacent amino acids of IL-2, which are to be transferred from IL-2 to IL-21. A stretch of amino acids may also encompass the entire number of adjacent amino acids of IL-2. Preferably, a stretch of amino acids of IL-2 to be substituted for amino acids of IL-21 is between about 3 and 60 adjacent amino acids, more preferably between about 5 and 40 adjacent amino acids and even more preferably between about 10 and 30 adjacent amino acids in length.

The amino acids of IL-2 to be transferred to IL-21 or substituted for IL-21 amino acids may be located at any position throughout the IL-2 molecule, e.g. at the N-terminus, at the C-terminus, or in the central portion. The amino acids of IL-2 to be transferred may comprise amino acids being positioned in any typical secondary or 3-dimensional protein structure like a helical portion, a beta-sheet, a beta-bridge, a bonded turn or a bend.

The number of IL-2 amino acids to be transferred to IL-21 may preferably be between about 0.5% and about 50% of all amino acids of IL-2, more preferably between about 5% and 40% of all amino acids of IL-2 and even more preferably between about 10% and 30% of all amino acids of IL-2. In a further embodiment, the number of substituted amino acids may be between about 1% and about 90% of one type of secondary structure as defined herein above of IL-2, preferably between about 1% and about 60% of helical structures of IL-2, more preferably between about 1% and about 60% of H structures or a helices of IL-2.

The amino acids to be transferred from IL-2 to IL-21 may be present in one or more stretches of amino acids of IL-2. Preferably, a transfer or substitution may be carried out with 1, 2, 4, 5, 6, 7, 8, 9, 10, 11, 12, 13, 14 or 15 stretches of amino acids of IL-2. The present invention also encompasses the substitution of more stretches of amino acids of IL-2, up to about 100.

Is more than one stretch of amino acids to be transferred from IL-2, the stretches may be either derived from positions in close proximity, e.g. only detached by one, two or three amino acids, or be derived from positions located at the opposite extremities of the IL-2 primary structure. Alternatively, the stretches of may be located anywhere throughout the IL-2 primary structure.

Are the stretches to be transferred to IL-21 located anywhere throughout the IL-2 primary structure, the stretches may or may not be located in close proximity in the secondary and/or tertiary structure of IL-2. Typically, the tertiary structure of IL-2, may be obtained via by crystallographic or NMR spectroscopic analyses. Alternatively, in further embodiments of the present invention some stretches of amino acids to be transferred from IL-2 to IL-21 may be grouped at different 3-dimensional positions or regions of IL-2, e.g. there may be one or more clusters of stretches of amino acids to be transferred in one or more 3-dimensional regions or positions of a protein.

In a further preferred embodiment of the present invention, the transferred amino acids or stretches of amino acids of IL-2 reside in portions of IL-2 which are responsible for the binding of IL-2 to its cognate receptor, preferably to the receptor complex of IL-2R and γc (see, for example, FIG. 2, which depicts an interaction between IL-2, IL-2Rα and IL-2Rβ and γc). The term “portions of IL-2 which are responsible for the binding of IL-2 to its cognate receptor” relates to 3-dimensional sections of human IL-2 which were implicated by crystallographic studies to be involved in the IL-2 receptor binding (Stauber D J et al. (2006) PNAS 103 (8):2788-93; and Rickert M et al. (2005) Science, 308 (5727), 1477-80). Examples of such portions of IL-2 being responsible for the binding of IL-2 to its cognate receptor are helical or interhelical sections of IL-2 which provide protrusions in crystallographic tests or assays. Preferably, the amino acids or stretches of amino acids to be substituted may reside in one or more spatially cooperative helical regions of IL-2. The term “spatially cooperative helical regions of IL-2” relates to portions of IL-2 which are derivable or predictable from IL-2 structural data as being especially conjoined in a 3-dimensional structure of the protein. The amino acids or stretches of amino acids to be transferred may comprise the entire spatially cooperative helical regions of IL-2 or between about 0.5% and 95% of the spatially cooperative helical regions of IL-2, preferably about 20 to 60% of the spatially cooperative helical regions of IL-2. Methods to define the spatially cooperative helical regions of IL-2 would be known to the person skilled in the art or can be derived from any suitable structural biology textbook. Specific details for the definition of the spatially cooperative helical regions of IL-2 may be derived from (Stauber D J et al. (2006) PNAS 103 (8):2788-93; and Rickert M et al. (2005) Science, 308 (5727), 1477-80).

In a preferred embodiment the amino acids or stretches of amino acids of IL-21 as defined herein above may be substituted by amino acids or stretches of amino acids IL-2 as defined in SEQ ID NO: 3.

In a further embodiment the present invention relates to a polynucleotide encoding a IL-21 protein or IL-21 variant as defined herein above. A polynucleotide encoding a IL-21 protein or IL-21 variant according to the present invention may contain alterations in the coding regions, non-coding regions, or both. For example, the polynucleotides encoding a IL-21 protein or IL-21 variant may contain alterations, which produce silent substitutions, additions, or deletions, but do not alter the properties or activities of the encoded polypeptide. Nucleotide variants produced by silent substitutions due to the degeneracy of the genetic code are preferred.

The term “polynucleotide” means any suitable nucleic acid known to the person skilled in the art. For instance, a polynucleotide according to the present invention may be composed of DNA, RNA, PNA, CNA, HNA, LNA or ANA. The DNA may be in the form of, e.g. A-DNA, B-DNA or Z-DNA. The RNA may be in the form of, e.g. p-RNA or structurally modified forms like hairpin RNA or a stem-loop RNA.

The term “PNA” relates to a peptide nucleic acid, i.e. an artificially synthesized polymer similar to DNA or RNA which is used in biological research and medical treatments, but which is not known to occur naturally. The PNA backbone is typically composed of repeating N-(2-aminoethyl)-glycine units linked by peptide bonds. The various purine and pyrimidine bases are linked to the backbone by methylene carbonyl bonds. PNAs are generally depicted like peptides, with the N-terminus at the first (left) position and the C-terminus at the right.

The term “CNA” relates to an aminocyclohexylethane acid nucleic acid. Furthermore, the term relates to a cyclopentane nucleic acid, i.e. a nucleic acid molecule comprising for example 2′-deoxycarbaguanosine.

The term “HNA” relates to hexitol nucleic acids, i.e. DNA analogues which are built up from standard nucleobases and a phosphorylated 1,5-anhydrohexitol backbone.

The term “LNA” relates to locked nucleic acids. Typically, a locked nucleic acid is a modified and thus inaccessible RNA nucleotide. The ribose moiety of an LNA nucleotide may be modified with an extra bridge connecting the 2′ and 4′ carbons. Such a bridge locks the ribose in a 3′-endo structural conformation. The locked ribose conformation enhances base stacking and backbone pre-organization.

The term “ANA” relates to arabinoic nucleic acids or derivatives thereof. A preferred ANA derivative in the context of the present invention is a 2′-deoxy-2′-fluoro-beta-D-arabinonucleoside (2′F-ANA).

The polynucleotide molecules according to the present invention may also comprise a combination of any one of DNA, RNA, PNA, CNA, HNA, LNA and ANA. Preferred polynucleotides are DNA or RNA molecules.

In preferred embodiment the polynucleotide encoding an IL-21 protein is the polynucleotide of SEQ ID NO: 13.

In another embodiment the IL-21 variant may comprise helical portions of IL-4. The term “Interleukin-4” or “IL-4” refers to a human Interleukin having the polypeptide sequence as defined in SEQ ID NO: 2. The term, thus, relates to a mature, processed, cleaved or secreted version of IL-4, which has been optimized for expression in mammal cells, as depicted in SEQ ID NO: 2. For certain purposes or uses of the invention, e.g. for the expression of the protein in vivo etc. an Interleukin-4 may additionally also be understood as comprising a signal sequence, preferably of additional 24 amino acids. This type of IL-4 is termed “precursor IL-4” and defined in SEQ ID NO: 6. Preferably, a “precursor IL-4” refers to a protein capable of being directed to the ER, secretory vesicles, or the extracellular space as a result of said signal sequence. If the IL-4 precursor is released into the extracellular space, the IL-4 precursor can undergo extracellular processing to produce “IL-4” or a “mature IL-4” or a “processed IL-4”, or a “cleaved IL-4”, or a “secreted IL-4” protein. Release into the extracellular space can occur by many mechanisms, including exocytosis and proteolytic cleavage. The terms “IL-4”, “mature IL-4”, “processed IL-4”, “cleaved IL-4” and “secreted IL-4” are used herein as synonyms and are to be understood, for the purpose of the present invention, as functionally equivalent. In specific embodiments of the present invention e.g. in the context of the expression of the protein or variant in bacterial systems, the term “Interleukin-4” or “IL-4” may also relate to a sequence of IL-4 as depicted in SEQ ID NO: 2 or derived therefrom as defined herein above or below, wherein at the N-terminus one additional amino acid is added that functions as a bacterial translation initiator. Preferably, the amino acid methionine may be added. A corresponding sequence is depicted in SEQ ID NO: 12.

In a more preferred embodiment the IL-21 variant may comprise between about 10 to 60% of the helical portions of IL-4 as defined in SEQ ID NO:2, more preferably between about 15 to 50%, even more preferably about 20%, 22%, 24%, 26%, 28%, 30%, 32%, 34%, 36%, 38%, 40%, 42%, 44%, 46% 48% or 50% of the helical portions of IL-4 as defined in SEQ ID NO:2. The term “helical portions of IL-4” refers to a helical secondary structure of IL-4 as defined herein above, preferably a G, H or I structure, more preferably an H structure or α helix. In a preferred embodiment of the present invention the helical portions of IL-4 may comprise helical regions A, B, C or D as defined in Hage T et al. (1999) Cell, 97 (2), 271-81. These helical portions may be present either alone or in any suitable combination, e.g. in a combination of helical regions A and B, A and C, A and D, or B and C or B and D, or C and D, or A and B and C, or B and C and D, or A and C and D, or A and B and C and D. The IL-21 protein may in a further preferred embodiment additionally comprise one or more interhelical regions of IL-4, e.g. the interhelical region between helical region A and B, the interhelical region between region B and C, the interhelical region between region C and D. The interhelical regions may be combined with any of the helical regions A, B, C or D. Preferably, the interhelical regions may be combined with the adjacent helical regions as defined in Hage T et al. (1999) Cell, 97 (2), 271-81. The term “interhelical region” as used herein above denotes any region between one of the helices as defined in Hage T et al. (1999) Cell, 97 (2), 271-81. An interhelical region may also comprise any subportion of the region between two helices, e.g. a stretch of amino acids at the conjunction to a helix and/or alternatively a stretch of amino acids at the center of the section between two helices. An interhelical region according to the present invention may comprise any of the non-helical secondary structures as defined by the DSSP method. An interhelical region may comprise any number of amino acids between two helices of IL-4 as defined in Hage T et al. (1999) Cell, 97 (2), 271-81. Preferably, the interhelical region may comprise between about 2 and 70 amino acids, more preferably about 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66 or 68 amino acids.

Typically, the substitution or replacement of IL-21 amino acids by helical regions and/or interhelical regions of IL-4 may be carried out based on a sequence alignment between IL-21 and IL-4. The term “sequence alignment” relates to any suitable alignment known to the person skilled in the art, e.g. a global sequence alignment as determinable using the FASTDB computer program based on the algorithm of Brutlag and colleagues (Comp. App. Biosci. 6 237-245 (1990)). The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter. Alternatively, the alignment may be carried out with amino acid sequences. Based on such an alignment identical or homologous sequences or sequence stretches may be defined. Preferably, helical portions of IL-4 may be inserted into IL-21 at positions of identity or homology between IL-4 and IL-21.

In a further preferred embodiment an IL-21 variant according to the present invention may comprise helix A of IL-4 and/or helix C of IL-4 as defined in Hage T et al. (1999) Cell, 97 (2), 271-81. In another embodiment an IL-21 variant may comprise helix A of IL-4 and/or helix C of IL-4 and additionally an interhelical portion between helix B and C of IL-4 as defined in Hage T et al. (1999) Cell, 97 (2), 271-81. In another embodiment an IL-21 variant may comprise helix A of IL-4 and helix C of IL-4 and additionally an interhelical region between helix B and C of IL-4 and an interhelical region between helix C and D of IL-4 as defined in Hage T et al. (1999) Cell, 97 (2), 271-81.

In another preferred embodiment the IL-21 variant is the IL-21/IL-4 hybrid of SEQ ID NO: 8.

In a further embodiment the present invention relates to a polynucleotide encoding a IL-21 variant comprising helical portions of IL-4 as defined herein above. A polynucleotide encoding an IL-21 variant comprising helical portions of IL-4 according to the present invention may contain alterations in the coding regions, non-coding regions, or both. For example, the polynucleotide encoding an IL-21 variant comprising helical portions of IL-4 may contain alterations, which produce silent substitutions, additions, or deletions, but do not alter the properties or activities of the encoded polypeptide. Nucleotide variants produced by silent substitutions due to the degeneracy of the genetic code are preferred.

In a preferred embodiment the polynucleotide encoding an IL-21 variant comprising helical portions of IL-4 is the polynucleotide of SEQ ID NO: 15.

In another embodiment the IL-21 variant may comprise helical portions of IL-2. The term “Interleukin-2” or “IL-2” refers to a human Interleukin having the polypeptide sequence as defined in SEQ ID NO:3. The term, thus, relates to a mature, processed, cleaved or secreted version of IL-2, which has been optimized for expression in mammal cells, as depicted in SEQ ID NO:3. For certain purposes or uses of the invention, e.g. for the expression of the protein in vivo etc. an Interleukin-2 may additionally also be understood as comprising a signal sequence, preferably of additional 20 amino acids. This type of IL-2 is termed “precursor IL-2” and defined in SEQ ID NO: 5. Preferably, a “precursor IL-2” refers to a protein capable of being directed to the ER, secretory vesicles, or the extracellular space as a result of said signal sequence. If the IL-2 precursor is released into the extracellular space, the IL-2 precursor can undergo extracellular processing to produce “IL-2” or a “mature IL-2” or a “processed IL-2”, or a “cleaved IL-2”, or a “secreted IL-2” protein. Release into the extracellular space can occur by many mechanisms, including exocytosis and proteolytic cleavage. The terms “IL-2”, “mature IL-2”, “processed IL-2”, “cleaved IL-2” and “secreted IL-2” are used herein as synonyms and are to be understood, for the purpose of the present invention, as functionally equivalent. In specific embodiments of the present invention e,g, in the context of the expression of the protein or variant in bacterial systems, the term “Interleukin-2” or “IL-2” may also relate to a sequence of IL-2 as depicted in SEQ ID NO:3 or derived therefrom as defined herein above or below, wherein at the N-terminus one additional amino acid is added that functions as a bacterial translation initiator. Preferably, the amino acid methionine may be added. A corresponding sequence is depicted in SEQ ID NO: 11.

In a more preferred embodiment the IL-21 variant may comprise between about 10 to 65% of the helical portions of IL-2 as defined in SEQ ID NO:3, more preferably between about 15 to 55%, even more preferably about 20%, 22%, 24%, 26%, 28%, 30%, 32%, 34%, 36%, 38%, 40%, 42%, 44%, 46% 48%, 50%, 52 or 54% of the helical portions of IL-2 as defined in SEQ ID NO: 3. The term “helical portions of IL-2” refers to a helical secondary structure of IL-2 as defined herein above, preferably a G, H or I structure, more preferably an H structure or α helix. In a preferred embodiment of the present invention the helical portions of IL-2 may comprise helical regions A, B, C or D as defined in Stauber D J et al. (2006) PNAS 103 (8),2788-93. These helical portions may be present either alone or in any suitable combination, e.g. in a combination of helical regions A and B, A and C, A and D, or B and C or B and D, or C and D, or A and B and C, or B and C and D, or A and C and D, or A and B and C and D. The IL-21 protein may in a further preferred embodiment additionally comprise one or more interhelical region of IL-2, e.g. the interhelical region between helical region A and B, the interhelical region between region B and C, the interhelical region between region C and D. The interhelical regions may be combined with any of the helical regions A, B, C or D. Preferably, the interhelical regions may be combined with the adjacent helical regions as defined in Stauber D J et al. (2006) PNAS 103 (8), 2788-93. The term “interhelical region” as used herein above denotes any region between one of the helices as defined in Stauber D J et al. (2006) PNAS 103 (8), 2788-93. An interhelical region may also comprise any subportion of the region between two helices, e.g. a stretch of amino acids at the conjunction to a helix and/or alternatively a stretch of amino acids at the center of the section between two helices. An interhelical region according to the present invention may comprise any of the non-helical secondary structures as defined by the DSSP method. An interhelical region may comprise any number of amino acids between two helices of IL-2 as defined in Stauber D J et al. (2006) PNAS 103 (8), 2788-93. Preferably, the interhelical region may comprise between about 2 and 70 amino acids, more preferably about 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66 or 68 amino acids.

Typically, the substitution or replacement of IL-21 amino acids by helical regions and/or interhelical regions of IL-2 may be carried out based on a sequence alignment between IL-21 and IL-2. The term “sequence alignment” relates to any suitable alignment known to the person skilled in the art, e.g. a global sequence alignment as determinable using the FASTDB computer program based on the algorithm of Brutlag and colleagues as defined herein above. Based on such an alignment identical or homologous sequences or sequence stretches may be defined.

Preferably, helical portions of IL-2 may be inserted into IL-21 at positions of identity or homology between IL-2 and IL-21.

In a further preferred embodiment an IL-21 variant according to the present invention may comprise helix A of IL-2 and/or helix C of IL-2 as defined in Stauber D J et al. (2006) PNAS 103 (8), 2788-93. In another embodiment an IL-21 variant may comprise helix A of IL-2 and/or helix C of IL-2 and additionally an interhelical portion between helix B and C of IL-2 as defined in Stauber D J et al. (2006) PNAS 103 (8), 2788-93. In another embodiment an IL-21 variant may comprise helix A of IL-2 and helix C of IL-2 and additionally an interhelical region between helix B and C of IL-2 and an interhelical region between helix C and D of IL-2 as defined in Stauber D J et al. (2006), PNAS 103 (8), 2788-93.

In another preferred embodiment the IL-21 variant is the IL-21/IL-2 hybrid of SEQ ID NO: 7.

In a further embodiment the present invention relates to a polynucleotide encoding a IL-21 variant comprising helical portions of IL-2 as defined herein above. A polynucleotide encoding a IL-21 variant comprising helical portions of IL-2 according to the present invention may contain alterations in the coding regions, non-coding regions, or both. For example, the polynucleotides encoding an IL-21 variant comprising helical portions of IL-2 may contain alterations, which produce silent substitutions, additions, or deletions, but do not alter the properties or activities of the encoded polypeptide. Nucleotide variants produced by silent substitutions due to the degeneracy of the genetic code are preferred.

In a preferred embodiment the polynucleotide encoding an IL-21 variant comprising helical portions of IL-2 is the polynucleotide of SEQ ID NO: 14.

In another embodiment of the present invention the IL-21 variant may comprise helical portions of IL-2 and IL-4. The helical portions of IL-2 and IL-4 may comprise any of the above mentioned helical portions or sections of IL-2 and IL-4. Preferably, the IL-21 variant may comprise between about 10 and 65% of the helical portions of IL-2 and IL-4. The term “between about 10 and 65% of the helical portions of IL-2 and IL-4” means that the amount of helical portions derived from IL-2 and IL-4 together is between about 10 and 65%. Within this amount of helical portions the fraction of helical portions derived from IL-2 may be between 1% and 99%. Likewise, the fraction of helical portions derived from IL-4 may be between 1% and 99%. Preferably the fraction of helical portions derived from IL-2 may be 40%, 45%, 50%, 55% or 60%; and accordingly the fraction of helical portions derived from IL-4 may be 40%, 45%, 50%, 55% or 60%.

In a further embodiment of the present invention the IL-21 protein or IL-21 variant, the IL-2 protein or the IL-4 protein as defined herein above may comprise or be fused to any marker sequence or purification tag known to the person skilled in the art. In a preferred embodiment, the marker amino acid sequence is a hexa-histidine peptide, such as the tag provided in a pQE vector (QIAGEN). As described by Gentz and coworkers (PNAS 86, 821-824 (1989)), for instance, hexa-histidine provides for convenient purification of the fusion protein. Another peptide tag useful for purification, the “HA” tag, corresponds to an epitope derived from the influenza hemagglutinin protein (Wilson, et al., (1984) Cell 37, 767). In a further preferred embodiment, the marker sequence may be a “FLAG” polypeptide. The FLAG antigenic polypeptide may be fused to an IL-21, IL-2 or IL-4 or an IL-21 variant polypeptide of the invention at either or both the amino or the carboxy terminus. In preferred embodiments, an IL-21-FLAG, IL-4-FLAG or IL-2-FLAG fusion protein may be expressed from a pFLAG-CMV-5a or a pFLAG-CMV-1 expression vector (Andersson S., et al. (1989) J. Biol. Chem., 264, 8222-29; Thomsen, D. R., et al., (1984) PNAS, 81, 659-63; and Kozak, M. (1984) Nature 308, 241). In further preferred embodiments, a-FLAG fusion protein is detectable by anti-FLAG monoclonal antibodies.

Interleukin proteins or variants of the invention, in particular IL-21 proteins or IL-21 variants, IL-2 or IL-4 can be produced recombinantly by any suitable method known to the person skilled in the art. The present invention, thus, also encompasses methods for the production of IL-21 proteins and IL-21 variants or IL-2 or IL-4.

Accordingly, the present invention contemplates vectors containing the polynucleotides encoding IL-21, IL-21 variants, IL-2 or IL-4 of the present invention as has been defined herein above, host cells, and the production of IL-21, IL-21 variants, IL-2 or IL-4 by recombinant techniques.

A suitable vector may be, for example, a phage, plasmid, viral, or retroviral vector. Retroviral vectors may be replication competent or replication defective.

In the latter case, viral propagation generally will occur only in complementing host cells.

Polynucleotides encoding IL-21, IL-21 variants, IL-2 or IL-4 may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells. The polynucleotide insert should be operatively linked to an appropriate promoter, such as the phage lambda PL promoter, the E. coli lac, trp, phoA and tac promoters, the SV40 early and late promoters and promoters of retroviral LTRs. Other suitable promoters are known to the person skilled in the art. The expression constructs may further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include, for instance, dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris); insect cells such as Drosophila melanogaster S2 and Spodoptera frugiperda Sf9 cells; animal cells such as CHO, COS, HEK 293, and Bowes melanoma cells; and plant cells. Appropriate culture mediums and conditions for the above described host cells are known in the art.

Vectors preferred for use in bacteria include, but are not limited to pQE70, pQE60 and pQE9, available from QIAGEN, Inc.; pBluescript vectors, Phagescript vectors, pNH8A, pNH16a, pNH18A, pNH46A, available from Stratagene Cloning Systems, Inc.; pKK223-3, pKK233-3, pDR540, pRIT5 available from Pharmacia Biotech, Inc., and pET vectors available from Novagen. Among preferred eukaryotic vectors are pWLNEO, pSV2CAT, pOG44, pXTI and pSG available from Stratagene; and pSVK3, pBPV, pMSG and pSVL available from Pharmacia. Preferred expression vectors for use in yeast systems include, but are not limited to pYES2, pYDI, pTEFI/Zeo, pYES2/GS, pPICZ, pGAPZ, pGAPZalph, pPIC9, pPIC3.5, pHIL-D2, pHIL-SI, pPIC3.5K, pPIC9K, and PAO815 (all available from Invitrogen, Inc.). Other suitable vectors are known to the person skilled in the art.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., (Basic Methods In Molecular Biology (1986)). It is specifically contemplated that IL-21, IL-21 variants, IL-2 or IL-4 of the present invention may in fact be expressed by a host cell lacking a recombinant vector.

An IL-21, IL-21 variant, IL-2 or IL-4 protein in accordance with the present invention can be recovered and purified from recombinant cell cultures by any suitable method known to the person skilled in the art, e.g. methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Preferably, high performance liquid chromatography (“HPLC”) can be employed for purification.

IL-21, IL-21 variants, IL-2 or IL-4 in accordance with the present invention can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides or proteins of the present invention may be glycosylated or may be non-glycosylated. In addition, polypeptides or proteins of the invention may also include an initial modified methionine residue, in some cases as a result of hostmediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked. Preferably, a sequence as depicted in SEQ ID NO: 10 may be used for the expression of IL-21 in a prokaryotic host, a sequence as depicted in SEQ ID NO: 12 may be used for the expression of IL-4 in a prokaryotic host or a sequence as depicted in SEQ ID NO: 11 may be used for the expression of IL-2 in a prokaryotic hosts.

In specific embodiment, the yeast Pichia pastoris is used to express IL-21, IL-21 variants, IL-2 or IL-4 according to the present invention in a eukaryotic system. Pichia pastoris is amethylotrophic yeast, which can metabolize methanol as its sole carbon source. A main step in the methanol metabolization pathway is the oxidation of methanol to formaldehyde using O₂. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O₂. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris (Ellis S B et al., (1985) Mol. Cell. Biol. 5, 1111-21; Loutz P J et al., (1989) Yeast 5, 167-77; Tschopp J F et al., (1987) Nucl. Acids Res. 15, 3859-76). Thus, a heterologous coding sequence, such as, for example, a polynucleotide encoding IL-21, IL-21 variants, IL-2 or IL-4, under the transcriptional regulation of all or part of the AOX1 regulatory sequence is expressed at exceptionally high levels in Pichia yeast grown in the presence of methanol.

In one example, the plasmid vector pPIC9K may be used to express DNA encoding a polypeptide of the invention, as set forth herein, in a Pichia yeast system essentially as described in “Pichia Protocols: Methods in Molecular Biology,” (D. R. Higgins and J. Gregg, eds. The Humana Press, Totowa, N.J., 1998). This expression vector allows expression and secretion of a protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, including, but not limited to pYES2, pYDI, pTEFI/Zeo, pYES2/GS, pPICZ, pGAPZ, pGAPZalpha, pPIC9, pPIC3.5, pHIL-D2, pHIL-SI, pPIC3.5K, and PA0815, as a person skilled in the art would know, as long as the proposed expression construct provides appropriately located signals for transcription, translation, secretion (if desired), and the like, including an in-frame AUG, as required.

In another embodiment, high-level expression of a heterologous coding sequence, such as, for example, a polynucleotide of the present invention, may be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein above, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides encoding IL-21, IL-21 variants, IL-2 or IL-4, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, resulting in the formation of a new transcription unit.

In addition, IL-21, IL-21 variants, IL-2 or IL-4 of the invention can be chemically synthesized using techniques known in the art (Creighton, (1983), Proteins: Structures and Molecular Principles, W. H. Freeman & Co., N. Y., and Hunkapiller et al., (1984) Nature, 310, 105-111). For example, a polypeptide corresponding to a variant or protein of the invention can be synthesized by use of a peptide synthesizer.

In a further aspect the present invention relates to a pharmaceutical composition comprising an IL-21 protein and IgA inducing protein (IGIP). In a further aspect the present invention relates to a pharmaceutical composition comprising an IL-21 protein and Syntenin-1. In a further aspect the present invention relates to a pharmaceutical composition comprising an IL-21 protein and Galectin-1. In yet another aspect the present invention relates to a pharmaceutical composition comprising an IL-21 protein and Galectin-3. In yet another aspect the present invention relates to a pharmaceutical composition comprising an IL-21 protein and IGIP and Syntenin-1. In yet another aspect the present invention relates to a pharmaceutical composition comprising an IL-21 protein and IGIP and Galectin-1. In yet another aspect the present invention relates to a pharmaceutical composition comprising an IL-21 protein and IGIP and Galectin-3. In yet another aspect the present invention relates to a pharmaceutical composition comprising an IL-21 protein and Syntenin-1 and Galectin-1. In yet another aspect the present invention relates to a pharmaceutical composition comprising an IL-21 protein and Syntenin-1 and Galectin-3. In yet another aspect the present invention relates to a pharmaceutical composition comprising an IL-21 protein and Galectin-1 and Galectin-3. In yet another aspect the present invention relates to a pharmaceutical composition comprising an IL-21 protein and IGIP and Syntenin-1 and Galectin-1. In yet another aspect the present invention relates to a pharmaceutical composition comprising an IL-21 protein and IGIP and Syntenin-1 and Galectin-3. In yet another aspect the present invention relates to a pharmaceutical composition comprising an IL-21 protein and IGIP and Galectin-1 and Galectin-3. In yet another aspect the present invention relates to a pharmaceutical composition comprising an IL-21 protein and Syntenin-1 and Galectin-1 and Galectin-3. In yet another aspect the present invention relates to a pharmaceutical composition comprising an IL-21 protein and IGIP and Syntenin-1 and Galectin-1 and Galectin-3. In preferred embodiment of the present invention any of said pharmaceutical compositions as described herein above comprises additionally to the IL-21 protein an IL-21 variant as mentioned herein above. In a further, particularly preferred embodiment of the present invention any of said pharmaceutical compositions as described herein above comprises instead of the IL-21 protein an IL-21 variant as mentioned herein above.

The term “IgA inducing protein (IGIP)” as used herein refers to a human protein or a species homologue of higher eukaryotic origin, preferably to the human protein as described in Endsley et al. (2009), The Journal of Immunology, 182: 1854-1859, more preferably to the protein having the Genbank accession No. NP_{—}001007190.1, GI:55770874, or being encoded by the nucleotide sequence having the Genbank accession No. NM_{—}001007189.1, or functional homologues thereof, e.g. proteins comprising deletions, modifications such as amino acid exchanges, additions etc. which are functionally comparable with the wildtype sequence, or isoforms thereof.

The term “Syntenin-1” as used herein refers to a human protein or a species homologue of higher eukaryotic origin, preferably to the human protein as described in Sira et al. (2009), International Immunology, 21(9): 1013-1023, more preferably to the protein having the Swiss-Prot accession No. 000560.1, GI:20455281, or Genbank Acc. No. BC013254, or functional homologues thereof, e.g. proteins comprising deletions, modifications such as amino acid exchanges, additions etc. which are functionally comparable with the wildtype sequence, or isoforms thereof.

The term “Galectin-1” as used herein refers to a human protein or a species homologue of higher eukaryotic origin, preferably to the human protein as described in Tsai et al. (2008), The Journal of Immunology, 181: 4570-4579, more preferably to the protein having the Genbank accession No. NP_{—}002296.1, GI:4504981, or being encoded by the nucleotide sequence having the Genbank accession No. NM_{—}002305.3, or functional homologues thereof, e.g. proteins comprising deletions, modifications such as amino acid exchanges, additions etc. which are functionally comparable with the wildtype sequence, or isoforms thereof.

The term “Galectin-3” as used herein refers to a human protein or a species homologue of higher eukaryotic origin, preferably to the human protein as described in van Stijn et al. (2009), Mol. Immunol., 46 (16): 3292-3299, more preferably to the protein having the Genbank accession No. NP_{—}002297.2, GI:115430223, or being encoded by the nucleotide sequence having the Genbank accession No. NM_{—}002306.2, or functional homologues thereof, e.g. proteins comprising deletions, modifications such as amino acid exchanges, additions etc. which are functionally comparable with the wildtype sequence, or isoforms thereof.

In a further aspect the present invention relates to a pharmaceutical composition for the treatment of a primary humoral immunodeficiency disease, comprising an IL-21 protein and IL-2. In a further aspect the present invention relates to a pharmaceutical composition for the treatment of a primary humoral immunodeficiency disease, comprising an IL-21 protein and IL-4. In a further aspect the present invention relates to a pharmaceutical composition for the treatment of a primary humoral immunodeficiency disease, comprising an IL-21 protein and IL-2 and IL-4. In a further aspect the present invention relates to a pharmaceutical composition for the treatment of a primary humoral immunodeficiency disease, comprising an IL-21 protein and IGIP. In a further aspect the present invention relates to a pharmaceutical composition for the treatment of a primary humoral immunodeficiency disease, comprising an IL-21 protein and Syntenin-1. In a further aspect the present invention relates to a pharmaceutical composition for the treatment of a primary humoral immunodeficiency disease, comprising an IL-21 protein and Galectin-1. In a further aspect the present invention relates to a pharmaceutical composition for the treatment of a primary humoral immunodeficiency disease, comprising an IL-21 protein and Galectin-3. In a further aspect the present invention relates to a pharmaceutical composition for the treatment of a primary humoral immunodeficiency disease, comprising an IL-21 protein and IL-2 and/or IL-4 and IGIP. In a further aspect the present invention relates to a pharmaceutical composition for the treatment of a primary humoral immunodeficiency disease, comprising an IL-21 protein and IL-2 and/or IL-4 and Syntenin-1. In a further aspect the present invention relates to a pharmaceutical composition for the treatment of a primary humoral immunodeficiency disease, comprising an IL-21 protein and IL-2 and/or IL-4 and Galectin-1. In a further aspect the present invention relates to a pharmaceutical composition for the treatment of a primary humoral immunodeficiency disease, comprising an IL-21 protein and IL-2 and/or IL-4 and Galectin-3. In a further aspect the present invention relates to a pharmaceutical composition for the treatment of a primary humoral immunodeficiency disease, comprising an IL-21 protein and IL-2 and/or IL-4 and IGIP and/or Syntenin-1 and/or Galectin-1 and/or Galectin-3. In preferred embodiment the present invention relates to a pharmaceutical composition for the treatment of a primary humoral immunodeficiency disease comprising an IL-21 variant as mentioned herein above and IL-4. In further preferred embodiment the present invention relates to a pharmaceutical composition for the treatment of a primary humoral immunodeficiency disease comprising an IL-21 variant as mentioned herein above and IL-2. In yet a further preferred embodiment the present invention relates to a pharmaceutical composition for the treatment of a primary humoral immunodeficiency disease comprising an IL-21 variant as mentioned herein above and IL-4 and IL-2. In yet a further preferred embodiment the present invention relates to a pharmaceutical composition for the treatment of a primary humoral immunodeficiency disease comprising an IL-21 variant as mentioned herein above and at least one compound selected from the group of IGIP, Syntenin-1, Galectin-1 and Galectin-3. In yet a further preferred embodiment the present invention relates to a pharmaceutical composition the treatment of a primary humoral immunodeficiency disease comprising an IL-21 variant as mentioned herein above and IL-4 and/or IL-2 and IGIP and/or Syntenin-1 and/or Galectin-1 and/or Galectin-3.

In another aspect, the present invention relates to the use of an IL-21 protein and IL-2 for the preparation of a pharmaceutical composition for the treatment of a primary humoral immunodeficiency disease. In a further aspect the present invention relates to the use of an IL-21 protein and IL-4 for the preparation of a pharmaceutical composition for the treatment of a primary humoral immunodeficiency disease. In a further aspect the present invention relates to the use of an an IL-21 protein and IL-2 and IL-4 for the preparation of a pharmaceutical composition for the treatment of a primary humoral immunodeficiency disease. In yet another aspect the present invention relates to the use of an IL-21 protein and IGIP for the preparation of a pharmaceutical composition for the treatment of a primary humoral immunodeficiency disease. In yet another aspect the present invention relates to the use of an IL-21 protein and Syntenin-1 for the preparation of a pharmaceutical composition for the treatment of a primary humoral immunodeficiency disease. In yet another aspect the present invention relates to the use of an IL-21 protein and Galectin-1 for the preparation of a pharmaceutical composition for the treatment of a primary humoral immunodeficiency disease. In yet another aspect the present invention relates to the use of an IL-21 protein and Galectin-3 for the preparation of a pharmaceutical composition for the treatment of a primary humoral immunodeficiency disease. In yet another aspect the present invention relates to the use of an IL-21 protein and IL-2 and/or IL-4 and IGIP for the preparation of a pharmaceutical composition for the treatment of a primary humoral immunodeficiency disease. In yet another aspect the present invention relates to the use of an IL-21 protein and IL-2 and/or IL-4 and Syntenin-1 for the preparation of a pharmaceutical composition for the treatment of a primary humoral immunodeficiency disease. In yet another aspect the present invention relates to the use of an IL-21 protein and IL-2 and/or IL-4 and Galectin-1 for the preparation of a pharmaceutical composition for the treatment of a primary humoral immunodeficiency disease. In yet another aspect the present invention relates to the use of an IL-21 protein and IL-2 and/or IL-4 and Galectin-3 for the preparation of a pharmaceutical composition for the treatment of a primary humoral immunodeficiency disease. In yet another aspect the present invention relates to the use of an IL-21 protein and IL-2 and/or IL-4 and IGIP and/or Syntenin-1 and/or Galectin-1 and/or Galectin-3 for the preparation of a pharmaceutical composition for the treatment of a primary humoral immunodeficiency disease. In preferred embodiment the present invention relates to the use of an IL-21 variant as mentioned herein above and IL-2 for the preparation of a pharmaceutical composition for the treatment of a primary humoral immunodeficiency disease. In further preferred embodiment the present invention relates to the use of an IL-21 variant as mentioned herein above and IL-4 for the preparation of a pharmaceutical composition for the treatment of a primary humoral immunodeficiency disease. In yet a further preferred embodiment the present invention relates to the use of an IL-21 variant as mentioned herein above and IL-2 and IL-4 for the preparation of a pharmaceutical composition for the treatment of a primary humoral immunodeficiency disease. In yet a further preferred embodiment the present invention relates to the use of of an IL-21 variant as mentioned herein above and at least one compound selected from the group of IGIP, Syntenin-1, Galectin-1 and Galectin-3 for the preparation of a pharmaceutical composition for the treatment of a primary humoral immunodeficiency disease. In a further preferred embodiment the present invention relates tot he use of an IL-21 variant as mentioned herein above and IL-4 and/or IL-2 and IGIP and/or Syntenin-1 and/or Galectin-1 and/or Galectin-3 for the preparation of a pharmaceutical composition for the treatment of a primary humoral immunodeficiency disease.

In another embodiment the present invention relates to a method of treatment of a primary humoral immunodeficiency disease comprising the administration of an IL-21 protein and IL-2 to a patient, the administration of an IL-21 protein and IL-4 to a patient, the administration of an IL-21 protein and IL-2 and IL-4 to a patient, the administration of an IL-21 variant as mentioned herein above and IL-2 to a patient, the administration of an IL-21 variant as mentioned herein above and IL-4 to a patient, the administration of an IL-21 variant as mentioned herein above and IL-2 and IL-4 to a patient, the administration of an IL-21 protein and IGIP to a patient, the administration of an IL-21 protein and Syntenin-1 to a patient, the administration of an IL-21 protein and Galectin-1 to a patient, the administration of an IL-21 and Galectin-3 to a patient, the administration of an IL-21 variant as mentioned herein above and IGIP to an patient, the administration of an IL-21 variant as mentioned herein above and Syntenin-1 to an patient, the administration of an IL-21 variant as mentioned herein above and Galectin-1 to a patient, the administration of an IL-21 variant as mentioned herein above and Galectin-3 to an patient, or the administration of an IL-21 protein or an IL-21 variant as mentioned herein above and IL-2 and/or IL-4 and IGIP and/or Syntenin-1 and/or Galectin-1 and/or Galectin-3 to a patient.

The term “primary humoral immunodeficiency disease” means a disease, or disorder resulting from inherited or spontaneous defects of the immune system. The term preferably refers to multiple isolated defects and combined disorders, e.g. humoral immune deficiencies, severe combined immunodeficiencies, and disorders resulting from phagocytic and complement defects. A “primary humoral immunodeficiency disease” may be diagnosed by a skilled person according to symptomatic definitions available in the art, e.g. based on the definitions provided by the European Society for Immunodeficiencies (www.esid.org). Typical diagnostic criteria for a primary immune deficiency in adults may comprise (i) four or more infections requiring antibiotics within one year (e.g. otitis, bronchitis, sinusitis, pneumonia); (ii) recurring infections or infection requiring prolonged antibiotic therapy; (iii) two or more severe bacterial infections (e.g. osteomyelitis, meningitis, septicemia, cellulites; (iv) two or more radiologically proven pneumonia within 3 years; (v) an infection with an unusual localization or an unusual pathogen; and (vi) a family history of primary immune deficiencies. Furthermore, a diagnosis of a primar humoral immunodeficiency disease may be based on the information and definitions provided in the “Primary Immunodeficiency Diseases. A Molecular & Cellular Approach” (2006); 2^{nd} edition; Hans D. Ochs, C. I. Edward Smith and Jennifer M. Puck.

In a preferred embodiment the present invention relates to a pharmaceutical composition for the treatment of a disease involving a reduction in the level of secreted IgG and/or IgA, comprising an IL-21 protein and IL-2. In a further aspect the present invention relates to a pharmaceutical composition for the treatment of a disease involving a reduction in the level of secreted IgG and/or IgA, comprising an IL-21 protein and IL-4. In a further aspect the present invention relates to a pharmaceutical composition for the treatment of a disease involving a reduction in the level of secreted IgG and/or IgA, comprising an IL-21 protein and IL-2 and IL-4 In preferred embodiment the present invention relates to a pharmaceutical composition for the treatment of a disease involving a reduction in the level of secreted IgG and/or IgA comprising an IL-21 variant as mentioned herein above and IL-4. In further preferred embodiment the present invention relates to a pharmaceutical composition for the treatment of a disease involving a reduction in the level of secreted IgG and/or IgA comprising an IL-21 variant as mentioned herein above and IL-2. In yet a further preferred embodiment the present invention relates to a pharmaceutical composition for the treatment of a disease involving a reduction in the level of secreted IgG and/or IgA comprising an IL-21 variant as mentioned herein above and IL-4 and IL-2. In a further preferred embodiment the present invention relates to a pharmaceutical composition for the treatment of a disease involving a reduction in the level of secreted IgG and/or IgA, comprising an IL-21 protein and/or an IL-21 variant as mentioned herein above and IGIP and/or Syntenin-1 and/or Galectin-1 and/or Galectin-3. In a further preferred embodiment the present invention relates to a pharmaceutical composition for the treatment of a disease involving a reduction in the level of secreted IgG and/or IgA, comprising an IL-21 protein and/or an IL-21 variant as mentioned herein above and IL-2 and/or IL-4 and IGIP and/or Syntenin-1 and/or Galectin-1 and/or Galectin-3.

In further embodiment, the present invention relates to the use of an IL-21 protein and IL-2 for the preparation of a pharmaceutical composition for the treatment of a disease involving a reduction in the level of secreted IgG and/or IgA. In a further aspect the present invention relates to the use of an IL-21 protein and IL-4 for the preparation of a pharmaceutical composition for the treatment of a disease involving a reduction in the level of secreted IgG and/or IgA. In a further aspect the present invention relates to the use of an an IL-21 protein and IL-2 and IL-4 for the preparation of a pharmaceutical composition for the treatment of a disease involving a reduction in the level of secreted IgG and/or IgA.

In preferred embodiment the present invention relates to the use of an IL-21 variant as mentioned herein above and IL-2 for the preparation of a pharmaceutical composition for the treatment of a disease involving a reduction in the level of secreted IgG and/or IgA. In further preferred embodiment the present invention relates to the use of an IL-21 variant as mentioned herein above and IL-4 for the preparation of a pharmaceutical composition for the treatment of a disease involving a reduction in the level of secreted IgG and/or IgA. In yet a further preferred embodiment the present invention relates to the use of an IL-21 variant as mentioned herein above and IL-2 and IL-4 for the preparation of a pharmaceutical composition for the treatment of a disease involving a reduction in the level of secreted IgG and/or IgA.

In a further preferred embodiment the present invention relates to the use of an IL-21 protein and/or an IL-21 variant as mentioned herein above and IGIP and/or Syntenin-1 and/or Galectin-1 and/or Galectin-3 for the preparation of a pharmaceutical composition for the treatment of a disease involving a reduction in the level of secreted IgG and/or IgA. In a further preferred embodiment the present invention relates to the use of an IL-21 protein and/or an IL-21 variant as mentioned herein above and IL-2 and/or IL-4 and IGIP and/or Syntenin-1 and/or Galectin-1 and/or Galectin-3 for the preparation of a pharmaceutical composition for the treatment of a disease involving a reduction in the level of secreted IgG and/or IgA, comprising.

In another embodiment the present invention relates to a method of treatment of a disease involving a reduction in the level of secreted IgG and/or IgA comprising the administration of an IL-21 protein and IL-2 to a patient, the administration of an IL-21 protein and IL-4 to a patient, the administration of an IL-21 protein and IL-2 and IL-4 to a patient, the administration of an IL-21 variant as mentioned herein above and IL-2 to a patient, the administration of an IL-21 variant as mentioned herein above and IL-4 to a patient, the administration of an IL-21 variant as mentioned herein above and IL-2 and IL-4 to a patient, the administration of an IL-21 protein and IGIP to a patient, the administration of an IL-21 protein and Syntenin-1 to a patient, the administration of an IL-21 protein and Galectin-1 to a patient, the administration of an IL-21 and Galectin-3 to a patient, the administration of an IL-21 variant as mentioned herein above and IGIP to an patient, the administration of an IL-21 variant as mentioned herein above and Syntenin-1 to an patient, the administration of an IL-21 variant as mentioned herein above and Galectin-1 to a patient, the administration of an IL-21 variant as mentioned herein above and Galectin-3 to an patient, or the administration of an IL-21 protein or an IL-21 variant as mentioned herein above and IL-2 and/or IL-4 and IGIP and/or Syntenin-1 and/or Galectin-1 and/or Galectin-3 to a patient.

The term “disease involving a reduction in the level of secreted IgG and/or IgA” means a disease, or disorder wherein the emission or throw-off of antibodies of the isotypes IgG or IgA, or of the isotypes IgG and IgA, or of at least one of the subclasses IgG1, IgG2, IgG3, IgG4, IgA1 or IgA2 by B-type lymphocytes is decreased in comparison to a healthy individual. Such a disease may, for example, be diagnosed by any suitable diagnostic means known to the person skilled in the art, preferably based on the tests as described herein above in the context of the testing of IL-21 variants. For instance, such a disease may be diagnosed based on the definitions provided by the European Society for Immunodeficiencies (www.esid.org). Furthermore, a diagnosis of a disease involving a reduction in the level of secreted IgG and/or IgA may be based on the information and definitions provided in the “Primary Immunodeficiency Diseases. A Molecular & Cellular Approach” (2006); 2^{nd} edition; Hans D. Ochs, C. I. Edward Smith and Jennifer M. Puck.

In a preferred embodiment of the present invention relates to a pharmaceutical composition for the treatment of a disease connected with a symptom of impairment or blocking of immunoglobulin isotype-switching, comprising an IL-21 protein and IL-2. In a further aspect the present invention relates to a pharmaceutical composition for the treatment of a disease connected with a symptom of impairment or blocking of immunoglobulin isotype-switching, comprising an IL-21 protein and IL-4. In a further aspect the present invention relates to a pharmaceutical composition for the treatment of a disease connected with a symptom of impairment or blocking of immunoglobulin isotype-switching, comprising an IL-21 protein and IL-2 and IL-4 In preferred embodiment the present invention relates to a pharmaceutical composition for the treatment of a disease connected with a symptom of impairment or blocking of immunoglobulin isotype-switching comprising an IL-21 variant as mentioned herein above and IL-4. In further preferred embodiment the present invention relates to a pharmaceutical composition for the treatment of a disease connected with a symptom of impairment or blocking of immunoglobulin isotype-switching comprising an IL-21 variant as mentioned herein above and IL-2. In yet a further preferred embodiment the present invention relates to a pharmaceutical composition for the treatment of a disease connected with a symptom of impairment or blocking of immunoglobulin isotype-switching comprising an IL-21 variant as mentioned herein above and IL-4 and IL-2. In a further preferred embodiment the present invention relates to a pharmaceutical composition for the treatment of a disease connected with a symptom of impairment or blocking of immunoglobulin isotype-switching, comprising an IL-21 protein and/or an IL-21 variant as mentioned herein above and IGIP and/or Syntenin-1 and/or Galectin-1 and/or Galectin-3. In a further preferred embodiment the present invention relates to a pharmaceutical composition for the treatment of a disease connected with a symptom of impairment or blocking of immunoglobulin isotype-switching, comprising an IL-21 protein and/or an IL-21 variant as mentioned herein above and IL-2 and/or IL-4 and IGIP and/or Syntenin-1 and/or Galectin-1 and/or Galectin-3.

In further embodiment, the present invention relates to the use of an IL-21 protein and IL-2 for the preparation of a pharmaceutical composition for the treatment of a disease connected with a symptom of impairment or blocking of immunoglobulin isotype-switching. In a further aspect the present invention relates to the use of an IL-21 protein and IL-4 for the preparation of a pharmaceutical composition for the treatment of a disease connected with a symptom of impairment or blocking of immunoglobulin isotype-switching In a further aspect the present invention relates to the use of an an IL-21 protein and IL-2 and IL-4 for the preparation of a pharmaceutical composition for the treatment of a disease connected with a symptom of impairment or blocking of immunoglobulin isotype-switching.

In preferred embodiment the present invention relates to the use of an IL-21 variant as mentioned herein above and IL-2 for the preparation of a pharmaceutical composition for the treatment of a disease connected with a symptom of impairment or blocking of immunoglobulin isotype-switching. In further preferred embodiment the present invention relates to the use of an IL-21 variant as mentioned herein above and IL-4 for the preparation of a pharmaceutical composition for the treatment of a disease connected with a symptom of impairment or blocking of immunoglobulin isotype-switching. In yet a further preferred embodiment the present invention relates to the use of an IL-21 variant as mentioned herein above and IL-2 and IL-4 for the preparation of a pharmaceutical composition for the treatment of a disease connected with a symptom of impairment or blocking of immunoglobulin isotype-switching.

In a further preferred embodiment the present invention relates to the use of an IL-21 protein and/or an IL-21 variant as mentioned herein above and IGIP and/or Syntenin-1 and/or Galectin-1 and/or Galectin-3 for the preparation of a pharmaceutical composition for the treatment of a disease connected with a symptom of impairment or blocking of immunoglobulin isotype-switching. In a further preferred embodiment the present invention relates to the use of an IL-21 protein and/or an IL-21 variant as mentioned herein above and IL-2 and/or IL-4 and IGIP and/or Syntenin-1 and/or Galectin-1 and/or Galectin-3 for the preparation of a pharmaceutical composition for the treatment of a disease connected with a symptom of impairment or blocking of immunoglobulin isotype-switching.

In another embodiment the present invention relates to a method of treatment of a disease connected with a symptom of impairment or blocking of immunoglobulin isotype-switching comprising the administration of an IL-21 protein and IL-2 to a patient, the administration of an IL-21 protein and IL-4 to a patient, the administration of an IL-21 protein and IL-2 and IL-4 to a patient, the administration of an IL-21 variant as mentioned herein above and IL-2 to a patient, the administration of an IL-21 variant as mentioned herein above and IL-4 to a patient, the administration of an IL-21 variant as mentioned herein above and IL-2 and IL-4 to a patient, the administration of an IL-21 protein and IGIP to a patient, the administration of an IL-21 protein and Syntenin-1 to a patient, the administration of an IL-21 protein and Galectin-1 to a patient, the administration of an IL-21 and Galectin-3 to a patient, the administration of an IL-21 variant as mentioned herein above and IGIP to an patient, the administration of an IL-21 variant as mentioned herein above and Syntenin-1 to an patient, the administration of an IL-21 variant as mentioned herein above and Galectin-1 to a patient, the administration of an IL-21 variant as mentioned herein above and Galectin-3 to an patient, or the administration of an IL-21 protein or an IL-21 variant as mentioned herein above and IL-2 and/or IL-4 and IGIP and/or Syntenin-1 and/or Galectin-1 and/or Galectin-3 to a patient.

The term “disease connected with a symptom of impairment or blocking of immunoglobulin isotype-switching” means a disease, or disorder wherein the molecular process of switching from one isotype class to the next isotype class is non-functional or at least functionally impaired. Typically, the isotype switching from IgM to IgG and/or IgA may be functionally impaired or blocked. The term comprises situations in which an impairment or blocking is due to molecular rearrangements and situations in which an impairment or blocking is due to cellular problems during signal transduction processes. Such a disease or disorder may be diagnosed by any suitable diagnostic means known to the person skilled in the art, preferably based on the tests as described herein above in the context of the testing of IL-21 variants.

In another more preferred embodiment of the present invention the primary humoral immunodeficiency disease or the disease involving a reduction in the level of secreted IgG and/or IgA as mentioned herein above may be a selective deficiency of IgA, termed IgAD, a common variable immunodeficiency, termed CVID, a selective deficiency of IgG subclasses, termed IgGsD, an immunodeficiency with increased IgM, termed hyper-IgM-syndrome, or an X-linked agammaglobulinaemia. The terms “selective deficiency of IgA”, “common variable immunodeficiency”, “selective deficiency of IgG subclasses”, “immunodeficiency with increased IgM” and “X-linked agammaglobulinaemia” are known to the person skilled in the art and can be derived, for example, from any qualified medical immunology textbook, e.g. from “Primary Immunodeficiency Diseases. A Molecular & Cellular Approach”. (2006) 2nd Edition; Hans D. Ochs, C. I. Edward Smith and Jennifer M. Puck; or from Virella

G et al. (2007), Informa HealthCare ed; or Kuby Immunology, Kindt T J et al. (2006), W. H. Freeman ed. Preferably, the terms refer to definitions of diseases as provided by the WHO, e.g. in chapter III of ICD-10 Version 2007 in the section “Diseases of the blood and blood-forming organs and certain disorders involving the immune mechanism”, in particular the subsection “Certain disorders involving the immune mechanism (D80-D89)”. More preferably, the terms relate to definitions as provided in sub-subsection D80, e.g. in D80.0, D80.1, D80.2, D80.3 or D80.5 of the ICD-10 Version 2007 (http://www.who.int/classifications/apps/icd/icd10online/?gd80.htm+d849).

The present invention relates accordingly in preferred embodiments to pharmaceutical compositions comprising an IL-21 protein and IL-4, an IL-21 protein and IL-2, an IL-21 protein and IL-2 and IL-4, an IL-21 variant as mentioned herein above and IL-2, an IL-21 variant as mentioned herein above and IL-4, an IL-21 variant as mentioned herein above and IL-2 and IL-4, an IL-21 protein and at least one compound selected from the group consisting of IGIP, Syntenin-1, Galectin-1 and Galectin, an IL-21 variant as mentioned herein above and at least one compound selected from the group consisting of IGIP, Syntenin-1, Galectin-1 and Galectin-3, an IL-21 protein and IL-2 and/or IL-4 and at least one compound selected from the group consisting of IGIP, Syntenin-1, Galectin-1 and Galectin, an IL-21 variant as mentioned herein above and IL-2 and/or IL-4 and at least one compound selected from the group consisting of IGIP, Syntenin-1, Galectin-1 and Galectin-3 for the treatment of a selective deficiency of IgA, a common variable immunodeficiency, a selective deficiency of IgG subclasses, an immunodeficiency with increased IgM, or an X-linked agammaglobulinaemia. The present invention relates in further preferred embodiments also accordingly to the use of an IL-21 protein and IL-4, an IL-21 protein and IL-2, an IL-21 protein and IL-2 and IL-4, an IL-21 variant as mentioned herein above and IL-2, an IL-21 variant as mentioned herein above and IL-4, an IL-21 variant as mentioned herein above and IL-2 and IL-4, an IL-21 protein and at least one compound selected from the group consisting of IGIP, Syntenin-1, Galectin-1 and Galectin, an IL-21 variant as mentioned herein above and at least one compound selected from the group consisting of IGIP, Syntenin-1, Galectin-1 and Galectin-3, an IL-21 protein and IL-2 and/or IL-4 and at least one compound selected from the group consisting of IGIP, Syntenin-1, Galectin-1 and Galectin, an IL-21 variant as mentioned herein above and IL-2 and/or IL-4 and at least one compound selected from the group consisting of IGIP, Syntenin-1, Galectin-1 and Galectin-3 for the preparation of a pharmaceutical composition for the treatment of a selective deficiency of IgA, a common variable immunodeficiency, a selective deficiency of IgG subclasses, an immunodeficiency with increased IgM, or an X-linked agammaglobulinaemia. Furthermore, in further preferred embodiments the present invention also relates accordingly to a method of treatment of a selective deficiency of IgA, a common variable immunodeficiency, a selective deficiency of IgG subclasses, an immunodeficiency with increased IgM, or an X-linked agammaglobulinaemia comprising the administration of an IL-21 protein and IL-2 to a patient, the administration of an IL-21 protein and IL-4 to a patient, the administration of an IL-21 protein and IL-2 and IL-4 to a patient, the administration of an IL-21 variant as mentioned herein above and IL-2 to a patient, the administration of an IL-21 variant as mentioned herein above and IL-4 to a patient, or the administration of an IL-21 variant as mentioned herein above and IL-2 and IL-4 to a patient, the administration of an IL-21 protein and IGIP to a patient, the administration of an IL-21 protein and Syntenin-1 to a patient, the administration of an IL-21 protein and Galectin-1 to a patient, the administration of an IL-21 and Galectin-3 to a patient, the administration of an IL-21 variant as mentioned herein above and IGIP to an patient, the administration of an IL-21 variant as mentioned herein above and Syntenin-1 to an patient, the administration of an IL-21 variant as mentioned herein above and Galectin-1 to a patient, the administration of an IL-21 variant as mentioned herein above and Galectin-3 to an patient, or the administration of an IL-21 protein or an IL-21 variant as mentioned herein above and IL-2 and/or IL-4 and IGIP and/or Syntenin-1 and/or Galectin-1 and/or Galectin-3 to a patient.

A pharmaceutical composition according to the present invention may be administered with the help of various delivery systems known to the person skilled in the art, e.g., encapsulation in liposomes, microparticles, microcapsules, recombinant cells capable of expressing the compound, receptor-mediated endocytosis (see, e.g., Wu and Wu, (1987) J. Biol. Chem. 262, 4429-4432), construction of a nucleic acid as part of a retroviral or other vector, etc. Methods of introduction may be topical, enteral or parenteral. The methods of introduction may include intradermal, intramuscular, intraperitoneal, intravenous, subcutaneous, intranasal, inhalational, epidural, and oral routes. The composition may be administered by any convenient route, for example by infusion or bolus injection, by absorption through epithelial or mucocutaneous linings (e.g., oral mucosa, rectal and intestinal mucosa, etc.) or by inhalation and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary or inhalational administration can be employed, e.g., via the use of an inhaler or nebulizer, and a concomitant formulation with an aerosolizing agent.

In a specific embodiment, it may be desirable to administer the pharmaceutical compounds or compositions of the invention locally to the area in need of treatment; this may be achieved by, for example, and not by way of limitation, local infusion, e.g. during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein of the invention, care must be taken to use materials to which the protein does not absorb.

A preferred method of local administration is by direct injection. Preferably, IL-21, IL-21 variants, IL-2 or IL-4 as defined herein above of the present invention may be complexed with a delivery vehicle to be administered by direct injection into or locally within the area of arteries. Administration of a composition locally within the area of arteries refers to injecting the composition centimeters and preferably, millimeters within arteries. Another method of local administration is to contact a pharmaceutical composition of the present invention in or around a surgical wound. For example, a patient can undergo surgery and the pharmaceutical composition can be coated on the surface of tissue inside the wound or the pharmaceutical composition can be injected into areas of tissue inside the wound.

For systemic administration, IL-21, IL-21 variants, IL-2,IL-4, IGIP, Syntenin-1, Galectin-1 or Galectin-3 according to the present invention can be complexed to a targeted delivery vehicle. Suitable delivery vehicles for use with systemic administration comprise liposomes comprising ligands for targeting the vehicle to a particular site. Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., (1992) PNAS, 189, 11277-11281).

Oral delivery can be performed by complexing IL-21, IL-21 variants, IL-2, IL-4 IGIP, Syntenin-1, Galectin-1 or Galectin-3 as defined herein above to a carrier capable of withstanding degradation by digestive enzymes in the gut of an animal. Examples of such carriers include plastic capsules or tablets, such as those known in the art. Topical delivery can be performed, for instance, by mixing a polynucleotide construct of the present invention with a lipophilic reagent (e.g., DMSO) that is capable of passing into the skin.

In another embodiment the pharmaceutical composition may be delivered directly to internal organs, body cavities and the like by use of imaging devices used to guide an injecting needle directly to the site of interest. The pharmaceutical composition may also be administered to disease sites at the time of surgical intervention.

In another embodiment, the pharmaceutical composition can be delivered in a vesicle, in particular a liposome (Langer (1990) Science 249, 1527-1533; Treat et al., (1989) in Liposomes in the Therapy of Infectious Disease and Cancer, Lopez-Berestein and Fidler (eds.), Liss, New York, pp. 353-365; Lopez-Berestein, ibid., pp. 317-327).

In yet another embodiment, the composition can be delivered in a controlled release system. In one embodiment, a pump may be used (Sefton, CRC (1987) Crit. Ref. Biomed. Eng. 14, 201; Buchwald et al. (1980) Surgery 88, 507; Saudek et al., (1989) N. Engl. J. Med. 321, 574). In another embodiment, polymeric materials can be used (see Medical Applications of Controlled

Release, Langer and Wise (1974) CRC Pres., Boca Raton, Fla.; Controlled Drug Bioavailability, Drug Product Design and Performance, Smolen and Ball (1984) Wiley, New York; Ranger and Peppas, J. (1983) Macromol. Sci. Rev. Macromol. Chem. 23, 61; see also Levy et al., (1985) Science 228, 190; During et al., (1989) Ann. Neurol. 25; 351; Howard et al., (1989) J. Neurosurg. 71, 105). In yet another embodiment, a controlled release system can be placed in proximity of a therapeutic target, e.g. the brain, lymphatic organs etc. thus requiring only a fraction of the systemic dose (see, e.g., Goodson (1984) in Medical Applications of Controlled Release, vol. 2, pp. 115-138).

Preferably the pharmaceutical composition is in a form, which is suitable for oral, local or systemic administration. In a preferred embodiment the pharmaceutical composition is administered locally, orally or systemically.

In a further embodiment the pharmaceutical composition comprises a therapeutically effective amount of IL-21, IL-21 variants, IL-2 and/or IL-4 and/or IGIP and/or Syntenin-1 and/or Galectin-1 and/or Galectin-3 and a pharmaceutically acceptable carrier. The term “pharmaceutically acceptable” means approved by a regulatory agency or other generally recognized pharmacopeia for use in animals, and more particularly in humans. The term “carrier” refers to a diluent, adjuvant, excipient, or vehicle with which the therapeutic is administered. Such a carrier is pharmaceutically acceptable, i.e. is non-toxic to a recipient at the dosage and concentration employed. It is preferably isotonic, hypotonic or weakly hypertonic and has a relatively low ionic strength, such as provided by a sucrose solution. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers. Suitable pharmaceutical excipients include starch, glucose, sucrose, gelatin, malt, rice, flour, chalk, silica gel, sodium stearate, glycerol monostearate, talc, sodium ion, dried skim milk, glycerol, propylene, glycol, water, ethanol and the like. The composition, if desired, can also contain minor amounts of wetting or emulsifying agents, or pH buffering agents. These compositions can take the form of, e.g., solutions, suspensions, emulsion, powders, sustained-release formulations and the like. Examples of suitable pharmaceutical carriers are described in “Remington's Pharmaceutical Sciences” by E. W. Martin. Some other examples of substances which can serve as pharmaceutical carriers are sugars, such as glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; calcium carbonate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; cranberry extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tabletting agents, stabilizers, anti-oxidants and preservatives, can also be present. It is also advantageous to administer the active ingredients in encapsulated form, e.g. as cellulose encapsulation, in gelatine, with polyamides, niosomes, wax matrices, with cyclodextrins or liposomally encapsulated.

Generally, the ingredients are supplied either separately or mixed together in unit dosage form, for example, as a dry lyophilised powder or water free concentrate in a hermetically sealed container such as an ampoule or sachette indicating the quantity of active agent.

In a specific embodiment, the pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical composition of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Preferably, the pharmaceutical composition is administered directly or in combination with an adjuvant. Adjuvants may be selected from the group consisting of a chloroquine, protic polar compounds, such as propylene glycol, polyethylene glycol, glycerol, EtOH, 1-methyl L-2-pyrrolidone or their derivatives, or aprotic polar compounds such as dimethylsulfoxide (DMSO), diethylsulfoxide, di-n-propylsulfoxide, dimethylsulfone, sulfolane, dimethylformamide, dimethylacetamide, tetramethylurea, acetonitrile or their derivatives. These compounds are added in conditions respecting pH limitations. The composition of the present invention can be administered to a vertebrate. “Vertebrate” as used herein is intended to have the same meaning as commonly understood by one of ordinary skill in the art. Particularly, “vertebrate” encompasses mammals, and more particularly humans.

The term “administered” means administration of a therapeutically effective dose of the aforementioned composition. By “therapeutically effective amount” is meant a dose that produces the effects for which it is administered, preferably this effect is induction and enhancement of secretion of IgG and/or IgA antibodies in a patient. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

The pharmaceutical composition may be used in both human therapy and veterinary therapy, preferably in human therapy. The compounds described herein having the desired therapeutic activity may be administered in a physiologically acceptable carrier to a patient, as described herein. Depending upon the manner of administration, the compounds may be formulated in a variety of ways as discussed below. The concentration of the therapeutically active compound in the formulation may vary from about 0.00001-100 wt %. For instance, IL-21 or the IL-21 variant as defined herein above may be used in a concentration (w/w) of 10-100 wt %, IL-4 in a concentration (w/w) of 0.1-10 wt %, IL-2 in a concentration (w/w) of 0.1-10 wt %, IGIP in a concentration (w/w) of 0.1-50 wt %, Syntenin-1 in a concentration (w/w) of 0.1-50 wt %, Galectin-1 in a concentration (w/w) of 0.1-50 wt %, Galectin-3 in a concentration (w/w) of 0.1-50 wt %, a monoclonal antibody against CD40 in a concentration (w/w) of 1-50 wt %, CD40 L in a concentration (w/w) of 1-50 wt %, C4BP in a concentration (w/w) of 1-50 wt %, BAFF in a concentration (w/w) of 1-20 wt %, LIGHT in a concentration (w/w) of 1-20 wt %, interferon-α (IFN-α) in a concentration (w/w) of 0.1-10 wt %, a vaccine protein antigen in a concentration (w/w) of 1-20 wt %, and a vaccine polysaccharide antigen in a concentration (w/w) of 1-20 wt % in a pharmaceutical composition of the present invention.

The concentration of the active ingredients or compounds of a pharmaceutical composition according to the present invention may be further adjusted to the intended dosage regimen, the intended usage duration, the exact amount and ratio of all ingredients of the composition and further factors and parameter known to the person skilled in the art.

The active agents or compounds according to the present invention may be administered alone or in combination with other treatments.

The administration of the pharmaceutical composition can be done in a variety of ways. The preferable route of administering is the topical route. More preferred is the administration via inhalation.

The pharmaceutical composition of the present invention can also comprise a preservative. Preservatives according to certain compositions of the invention include, without limitation: butylparaben; ethylparaben; imidazolidinyl urea; methylparaben; O-phenylphenol; propylparaben; quaternium-14; quaternium-15; sodium dehydroacetate; zinc pyrithione; and the like.

The preservatives are used in amounts effective to prevent or retard microbial growth. Generally, the preservatives are used in amounts of about 0.1% to about 1% by weight of the total composition with about 0.1% to about 0.8% being preferred and about 0.1% to about 0.5% being most preferred.

Topical administration of the pharmaceutical composition of the present invention is useful when the desired treatment involves areas or organs readily accessible by topical administration. For a topically application, e.g. to the skin, mucous membrane, the pharmaceutical composition is preferably formulated with a suitable paste, ointment, lotion, cream, gel or transdermal patches.

The pharmaceutical preparations can, depending on the field of use, also be in the form of a foam, gel spray, mousse, suspensions or powder.

A suitable paste comprises the active ingredient suspended in a carrier. Such carriers include, but are not limited to, petroleum, soft white paraffin, yellow petroleum jelly and glycerol.

The pharmaceutical composition may also be formulated with a suitable ointment comprising the active components suspended or dissolved in a carrier. Such carriers include, but are not limited to, one or more of glycerol, mineral oil, liquid oil, liquid petroleum, white petroleum, yellow petroleum jelly, propylene glycol, alcohols, triglycerides, fatty acid esters such as cetyl ester, polyoxyethylene polyoxypropylene compound, waxes such as white wax and yellow beeswax, fatty acid alcohols such as cetyl alcohol, stearyl alcohol and cetylstearylalcohol, fatty acids such as stearic acid, cetyl stearate, lanolin, magnesium hydroxide, kaolin and water.

Alternatively, the pharmaceutical composition may also be formulated with a suitable lotion or cream comprising the active components suspended or dissolved in a carrier. Such carriers include, but are not limited to, one or more of mineral oil such as paraffin, vegetable oils such as castor oil, castor seed oil and hydrogenated castor oil, sorbitan monostearat, polysorbat, fatty acid esters such as cetyl ester, wax, fatty acid alcohols such as cetyl alcohol, stearyl alcohol, 2-octyldodecanol, benzyl alcohol, alcohols, triglycerides and water.

Alternatively, the pharmaceutical composition may also be formulated with a suitable gel comprising the active components suspended or dissolved in a carrier. Such carriers include, but are not limited to, one or more of water, glycerol, propyleneglycole, liquid paraffin, polyethylene, fatty oils, cellulose derivatives, bentonite and colloidal silicon dioxide.

The preparations according to the invention may generally comprise further auxiliaries as are customarily used in such preparations, e.g. preservatives, perfumes, antifoams, dyes, pigments, thickeners, surface-active substances, emulsifiers, emollients, finishing agents, fats, oils, waxes or other customary constituents, of a cosmetic or dermatological formulation, such as alcohols, polyols, polymers, foam stabilizers, solubility promoters, electrolytes, organic acids, organic solvents, or silicone derivatives.

The pharmaceutical composition according to the invention may comprise emollients. Emollients may be used in amounts, which are effective to prevent or relieve dryness. Useful emollients include, without limitation: hydrocarbon oils and waxes; silicone oils; triglyceride esters; acetoglyceride esters; ethoxylated glyceride; alkyl esters; alkenyl esters; fatty acids; fatty alcohols; fatty alcohol ethers; etheresters; lanolin and derivatives; polyhydric alcohols (polyols) and polyether derivatives; polyhydric alcohol (polyol) esters; wax esters; beeswax derivatives; vegetable waxes; phospholipids; sterols; and amides.

Thus, for example, typical emollients include mineral oil, especially mineral oils having a viscosity in the range of 50 to 500 SUS, lanolin oil, mink oil, coconut oil, cocoa butter, olive oil, almond oil, macadamia nut oil, aloe extract, jojoba oil, safflower oil, corn oil, liquid lanolin, cottonseed oil, peanut oil, purcellin oil, perhydrosqualene (squalene), caster oil, polybutene, odorless mineral spirits, sweet almond oil, avocado oil, calophyllum oil, ricin oil, vitamin E acetate, olive oil, mineral spirits, cetearyl alcohol (mixture of fatty alcohols consisting predominantly of cetyl and stearyl alcohols), linolenic alcohol, oleyl alcohol, octyl dodecanol, the oil of cereal germs such as the oil of wheat germ cetearyl octanoate (ester of cetearyl alcohol and 2-ethylhexanoic acid), cetyl palmitate, diisopropyl adipate, isopropyl palmitate, octyl palmitate, isopropyl myristate, butyl myristate, glyceryl stearate, hexadecyl stearate, isocetyl stearate, octyl stearate, octylhydroxy stearate, propylene glycol stearate, butyl stearate, decyl oleate, glyceryl oleate, acetyl glycerides, the octanoates and benzoates of (C12-C15) alcohols, the octanoates and decanoates of alcohols and polyalcohols such as those of glycol and glycerol, and ricin-oleates of alcohols and poly alcohols such as those of isopropyl adipate, hexyl laurate, octyl dodecanoate, dimethicone copolyol, dimethiconol, lanolin, lanolin alcohol, lanolin wax, hydrogenated lanolin, hydroxylated lanolin, acetylated lanolin, petrolatum, isopropyl lanolate, cetyl myristate, glyceryl myristate, myristyl myristate, myristyl lactate, cetyl alcohol, isostearyl alcohol stearyl alcohol, and isocetyl lanolate, and the like.

Moreover, the pharmaceutical composition according to the invention may also comprise emulsifiers. Emulsifiers (i.e., emulsifying agents) are preferably used in amounts effective to provide uniform blending of ingredients of the composition. Useful emulsifiers include (i) anionics such as fatty acid soaps, e.g., potassium stearate, sodium stearate, ammonium stearate, and triethanolamine stearate; polyol fatty acid monoesters containing fatty acid soaps, e.g., glycerol monostearate containing either potassium or sodium salt; sulfuric esters (sodium salts), e.g., sodium lauryl 5 sulfate, and sodium cetyl sulfate; and polyol fatty acid monoesters containing sulfuric esters, e.g., glyceryl monostearate containing sodium lauryl surfate; (ii) cationics chloride such as N(stearoyl colamino formylmethyl)pyridium; N-soya-N-ethyl morpholinium ethosulfate; alkyl dimethyl benzyl ammonium chloride; diisobutylphenoxytheoxyethyl dimethyl benzyl ammonium chloride; and cetyl pyridium chloride; and (iii) nonionics such as polyoxyethylene fatty alcohol ethers, e.g., monostearate; polyoxyethylene lauryl alcohol; polyoxypropylene fatty alcohol ethers, e.g., propoxylated oleyl alcohol; polyoxyethylene fatty acid esters, e.g., polyoxyethylene stearate; polyoxyethylene sorbitan fatty acid esters, e.g., polyoxyethylene sorbitan monostearate; sorbitan fatty acid esters, e.g., sorbitan; polyoxyethylene glycol fatty acid esters, e.g., polyoxyethylene glycol monostearate; and polyol fatty acid esters, e.g., glyceryl monostearate and propylene glycol monostearate; and ethoxylated lanolin derivatives, e.g., ethoxylated lanolins, ethoxylated lanolin alcohols and ethoxylated cholesterol. The selection of emulsifiers is exemplary described in Schrader, Hüthig Buch Verlag, Heidelberg, 2^{nd} edition, 1989, 3^{rd} part.

The pharmaceutical composition according to the invention may also include a surfactant. Suitable surfactants may include, for example, those surfactants generally grouped as cleansing agents, emulsifying agents, foam boosters, hydrotropes, solubilizing agents, suspending agents and nonsurfactants (facilitates the dispersion of solids in liquids).

The surfactants are usually classified as amphoteric, anionic, cationic and nonionic surfactants. Amphoteric surfactants include acylamino acids and derivatives and N-alkylamino acids. Anionic surfactants include: acylamino acids and salts, such as, acylglutamates, acylpeptides, acylsarcosinates, and acyltaurates; carboxylic acids and salts, such as, alkanoic acids, ester carboxylic acids, and ether carboxylic acids; sulfonic acids and salts, such as, acyl isethionates, alkylaryl sulfonates, alkyl sulfonates, and sulfosuccinates; sulfuric acid esters, such as, alkyl ether sulfates and alkyl sulfates. Cationic surfactants include: alkylamines, alkyl imidazolines, ethoxylated amines, and quaternaries (such as, alkylbenzyldimethylammonium salts, alkyl betaines, heterocyclic ammonium salts, and tetra alkylammonium salts). And nonionic surfactants include: alcohols, such as primary alcohols containing 8 to 18 carbon atoms; alkanolamides such as alkanolamine derived amides and ethoxylated amides; amine oxides; esters such as ethoxylated carboxylic acids, ethoxylated glycerides, glycol esters and derivatives, monoglycerides, polyglyceryl esters, polyhydric alcohol esters and ethers, sorbitan/sorbitol esters, and triesters of phosphoric acid; and ethers such as ethoxylated alcohols, ethoxylated lanolin, ethoxylated polysiloxanes, and propoxylated polyoxyethylene ethers.

Furthermore, a pharmaceutical composition according to the invention may also comprise a film former. Suitable film formers which are used in accord with the invention keep the composition smooth and even and include, without limitation: acrylamide/sodium acrylate copolymer; ammonium acrylates copolymer; Balsam Peru; cellulose gum; ethylene/maleic anhydride copolymer; hydroxyethylcellulose; hydroxypropylcellulose; polyacrylamide; polyethylene; polyvinyl alcohol; pvm/MA copolymer (polyvinyl methylether/maleic anhydride); PVP (polyvinylpyrrolidone); maleic anhydride copolymer such as PA-18 available from Gulf Science and Technology; PVP/hexadecene copolymer such as Ganex V-216 available from GAF Corporation; acryliclacrylate copolymer; and the like.

Generally, film formers can be used in amounts of about 0.1% to about 10% by weight of the total composition with about 1% to about 8% being preferred and about 0.1 DEG/O to about 5% being most preferred. Humectants can also be used in effective amounts, including: fructose; glucose; glulamic acid; glycerin; honey; maltitol; methyl gluceth-10; methyl gluceth-20; propylene glycol; sodium lactate; sucrose; and the like.

Other ingredients which can be added or used in a pharmaceutical composition according to the invention in amounts effective for their intended use, include: biological additives to enhance performance or consumer appeal such as amino acids, proteins, vanilla, aloe extract, bioflavinoids, and the like; buffering agents, chelating agents such as EDTA; emulsion stabilizers; pH adjusters; opacifying agents; and propellants such as butane carbon clioxide, ethane, hydrochlorofluorocarbons 22 and 142b, hydrofluorocarbon 152a, isobutane, isopentane, nitrogen, nitrous oxide, pentane, propane, and the like.

Furthermore, the preparations according to the invention may also comprise compounds, which have an antioxidative, free-radical scavenger, antierythematous, antiinflammatory or antiallergic action, in order to supplement or enhance their action. In particular, these compounds can be chosen from the group of vitamins, plant extracts, alpha- and beta-hydroxy acids, ceramides, antiinflammatory, antimicrobial or UV-filtering substances, and derivatives thereof and mixtures thereof. The lipid phase is advantageously chosen from the group of substances of mineral oils, mineral waxes, branched and/or unbranched hydrocarbons and hydrocarbon waxes, triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures; oils, fats or waxes, esters of saturated and/or unsaturated, branched and/or unbranched C₃-C₃₀-alkane carboxylic acids and saturated and/or unsaturated, branched and/or unbranched C₃-C₃₀-alcohols, from aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil, alkyl benzoates or silicone oils, such as, for example, cyclomethicone, dimethylpolysiloxane, diethylpolysiloxane, octamethylcyclo-tetrasiloxane and mixtures thereof or dialkyl ethers.

In a preferred embodiment of the present invention, the pharmaceutical composition may be administered via inhalation. The pharmaceutical preparations can accordingly be in the form of a spray, e.g. a pump spray or an aerosol. Typically, aerosols according to the present invention comprise the medicament or pharmaceutical composition, one or more chlorofluorocarbon propellants and either a surfactant or a solvent, such as ethanol. For instance aerosol propellants like propellant 11 and/or propellant 114 and/or propellant 12 may be used. Further suitable propellants for aerosols according to the invention are propane, butane, pentane and others. Additional propellants which may be used and which are believed to have minimal ozone-depleting effects in comparison to conventional chlorofluorocarbons comprise fluorocarbons and hydrogen-containing chlorofluorocarbons. Additional aerosols for medicinal aerosol formulations are disclosed in, for example, EP 0372777, WO 91/04011, WO 91/11173, WO 91/11495 and WO 91/14422. Typically, one or more adjuvants such as alcohols, alkanes, dimethyl ether, surfactants (including fluorinated and non-fluorinated surfactants, carboxylic acids, polyethoxylates etc) and conventional chlorofluorocarbon propellants in small amounts may be added to the formulations.

Further preferred is the use of 1,1,1,2-tetrafluoroethane in combination with both a cosolvent having greater polarity than 1,1,1,2-tetrafluoroethane (e.g. an alcohol or a lower alkane) and a surfactant in order to achieve a stable formulation of a pharmaceutical composition powder. Additionally, surfactants may be used as important components of aerosol formulations, in order to reduce the aggregation of the pharmaceutical composition and to lubricate, e.g. valves of a dispersing apparatus, if employed according to a further preferred embodiment of the present invention, thereby ensuring consistent reproducibility of valve actuation and accuracy of dose dispensed. Typically, the pharmaceutical composition according to the present invention may be pre-coated with surfactant prior to dispersal in 1,1,1,2-tetrafluoroethane.

In a further preferred embodiment of the present invention a pharmaceutical aerosol formulation may be dispersed with any suitable apparatus known to the person skilled in the art, preferably through a metered dose inhaler (MDI), a nebulizer, Rotahaler or an autohaler apparatus.

In another preferred embodiment of the present invention the alveolare resorption of the active compounds of the pharmaceutical composition according to the present invention may be increased via an optimization of the aerosol, e.g. by using a suitable aerosol production system, preferably by using the AERx aerosol delivery system as described in J. Schuster et al. (1997) Pharm. Res. 14, 354-357, or any suitable modification thereof.

In another preferred embodiment of the present invention the epithelial uptake of the active compounds of the pharmaceutical composition according to the present invention may be increased via a suitable coating of the components, e.g. a liposomal coating of the active compounds, preferably according to the guidance provided by Ten R M et al. (2002) Int Immunopharmacol. 2 (2-3), 333-44.

In another preferred embodiment of the present invention the active components of the pharmaceutical composition as defined herein above may be fused to a suitable carrier protein, e.g. to Ig Fc receptor proteins or polymeric Ig receptors. Preferably IL-21 protein or IL-variants as defined herein above may be provided as fusion proteins. The fusion partner may be provided at the N- or C-terminus. Also IL-2, IL-4, IGIP, Syntenin-1, Galectin-1, Galectin-3, a stimulator of CD40 molecules, preferably an anti-CD40 antibody, a CD40 ligand (CD40L) or C4BP; a ligand of the tumor necrosis superfamily, preferably BAFF or LIGHT; a polypeptide with human leukocyte interferon activity, preferably Interferon-α (IFN-α) and/or a vaccine protein antigen may be provided as fusion proteins, preferably as fusion proteins with Ig Fc receptor proteins or polymeric Ig receptors. Particularly preferred are fusions with neonatal constant region fragment (Fc) receptor (FcRn), e.g. as described in A. J. Bitonti et al. (2004) PNAS 101, 9763-9768. Further particularly preferred are fusions with the polymeric immunoglobulin receptor pIgR, e.g. as described in Ferkol T et al. (2000) Am J Respir Crit Care Med. 161 (3 Pt 1), 944-51.

It is further envisaged by the present invention that the optimization of aerosol techniques as defined herein above, the employment of suitable coatings as defined herein above and the fusion of the active compounds of the pharmaceutical composition of the present invention to suitable carrier proteins as defined herein above may be combined, either individually or as a group.

In a further preferred embodiment of the present invention the ratio between ingredients in the pharmaceutical composition or medicament may be suitably adjusted according to the skilled person's knowledge. For instance the ratio between IL-21 and IL-4 in a pharmaceutical composition may be adjusted to be between about 1:1 and 100:1, preferably between about 5:1 and 25:1, more preferably about 20:1. Alternatively, the ratio between an IL-21 variant as defined herein above and IL-4 in a pharmaceutical composition may be adjusted to be between about 1:1 and 100:1, preferably between about 5:1 and 25:1, more preferably to be about 20:1.

In a further preferred embodiment the ratio between IL-21 and IL-2 in a pharmaceutical composition may be adjusted to be between about 1:1 and 80:1, preferably between about 5:1 and 20:1, more preferably to be about 15:1. Alternatively, the ratio between an IL-21 variant as defined herein above and IL-2 in a pharmaceutical composition may be adjusted to be between about 1:1 and 80:1, preferably between about 5:1 and 20:1, more preferably to be about 15:1.

Assays, e.g. those based on tests described herein above or derivable from known and qualified textbooks of the prior art, may optionally be employed to help identify optimal ratios and/or dosage ranges for ingredients of pharmaceutical compositions of the present invention. The precise dose and the ratio between the ingredients of the pharmaceutical composition as defined herein above to be employed in the formulation will also depend on the route of administration, and the exact type of disease or disorder, and should be decided according to the judgment of the practitioner and each patient's circumstances. Effective doses or ingredient ratios may be extrapolated from dose-response curves derived from in vitro or (animal) model test systems.

Typically, the attending physician and clinical factors may determine the dosage regimen. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg; however, doses below or above this exemplary range are envisioned, especially considering the aforementioned factors.

In a further preferred embodiment of the present invention the pharmaceutical composition as defined herein above may further comprise additional factors able to enhance or improve the secretion of IgG and/or IgA antibodies from B-cells.

In a particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 at least one stimulator of CD40 molecules. The term “stimulator of CD40 molecules” as used herein above denotes any molecule which is capable of stimulating a CD40 molecule on a cell surface and correspondingly lead to a stimulation or activation of a CD40 dependent signal transduction cascade in the CD40 molecule expressing or displaying cell. It is commonly known that CD40 is a co-stimulatory protein found, inter alia, on antigen presenting cells and is required for their activation. Typically, upon binding of a stimulator of CD40 molecules to the CD40 molecule or CD40 receptor, e.g. on a B cell, a reaction is induced which includes, inter alia, the resting of B cell activation and the production of IL-4. As a result of these interactions, the B cell may undergo division, antibody isotype switching, and differentiation to plasma cells. The “stimulator of CD40 molecules” preferably relates to agonistic CD40 binding molecules, more preferably to an anti-CD40 antibody, a CD40 ligand or a protein having C4BP functionality. The term “anti-CD40 antibody” as used herein above refers to a monoclonal or polyclonal antibody against or specific for CD40, preferably an agonistic antibody mimicking the effect of the natural CD40 ligand and/or inducing most of its biological downstream effects. The term may also encompass any fragments, derivatives or modifications of such an antibody, e.g. single chain fragments etc., as long as the binding capability as defined herein above is not compromised or as long as the molecule is still able to bind CD40.

The term “CD40 ligand” as used herein above refers to a natural ligand of CD40, preferably the CD40 ligand (CD40 L) as defined by OMIM*300386. The term also comprises functional equivalents of CD40 L. The term “functional equivalent of CD40 L” refers to a protein which provides the function of CD 40 L, in particular in terms of binding to CD40. Such functional equivalents may be, for example, truncated versions of CD40 L, modified versions of CD40 L or CD40 L mimicking molecules.

The term “protein having C4BP functionality” refers to C4BP itself as well as functional equivalents thereof, e.g. truncated or modified versions of C4BP or molecules mimicking C4BP in terms of binding to CD40. Preferably, the term relates to C4BP as defined by OMIM*120830.

A stimulator of CD40 molecules as used herein above may be of any origin, in particular of mammalian origin, e.g. derived from a mouse, monkey or rat, preferably of human origin. The protein may be purified from natural sources or be produced recombinantly, e.g. in bacterial or lower eukaryotic host cells.

In a more preferred embodiment of the present invention a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 a monoclonal antibody against CD40, CD40 Las defined by OMIM*300386 or C4BP as defined by OMIM*120830.

In a further preferred embodiment two or more different stimulators of CD40 molecules as defined herein above may be used in combination, e.g. a monoclonal antibody against CD40 and CD40 L as defined by OMIM*300386 and C4BP as defined by OMIM*120830 may be used in combination in the context of a pharmaceutical composition of the present invention.

In a further particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 at least one ligand of the tumor necrosis factor superfamily.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules, at least one ligand of the tumor necrosis factor superfamily.

The term “ligand of the tumor necrosis superfamily” as used herein above refers to a member of a group of cytokines hat can cause apoptosis, preferably it refers to a member of the group comprising TNF alpha, FasL, TRAIL, LTB or lymphotoxin beta, LTA or lymphotoxin alpha or TNF beta, TNFSF8 or CD30L or CD153, TNFSF7 or CD70 or CD27L, TNFSF4 or OX40L or gp34, TNFSF9 or 4-1BBL, TNFSF11 or RANKL or TRANCE or ODF or OPGL, TNFSF15 or TL1, TNFSF12 or TWEAK or Apo3L, TNFSF14 or LIGHT or HVEM-L, TNFSF13 or APRIL or TRDL-1 TNFSF13B or BAFF or BLyS or THANK or TALL-1, TNFSF18 or TL6 or AITRL or GITRL, ED1 or EDA or Tabby, Tag7 and Eiger. The term also refers to functional equivalents of any of these factors. The term “functional equivalent” means a molecule which may be derived from the original factor or be modified based on the original factor, but still provides the core function of the original factor, in particular with regard to binding to a corresponding receptor molecule or the induction of typical biological effects, as would be known to the person skilled in the art, or to molecules mimicking the core function of the original factor. A ligand of the tumor necrosis superfamily may be of any origin, in particular of mammalian origin, e.g. derived from a mouse, monkey or rat, preferably of human origin. The protein may be purified from natural sources or be produced recombinantly, e.g. in bacterial or lower eukaryotic host cells.

Preferred members of the groups of ligands of the tumor necrosis superfamily are BAFF and LIGHT. The term “BAFF” relates preferably to human BAFF as defined by OMIM*603969. The term “LIGHT” relates preferably to human LIGHT as defined by OMIM*604520.

In a more preferred embodiment of the present invention a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 BAFF as defined by OMIM*603969 or LIGHT as defined by OMIM*604520.

In a further preferred embodiment two or more different ligands of the tumor necrosis superfamily as defined herein above may be used in combination, e.g. BAFF as defined by OMIM*603969 and LIGHT as defined by OMIM*604520 and APRIL may be used in combination in the context of a pharmaceutical composition of the present invention.

These factors may also be used in combination with pharmaceutical compositions comprising in addition to IL-21 and/or an IL-21 variant as defined herein above and IL-2 and/or IL-4 and/or IGIP, Syntenin-1, Galectin-1 or Galectin-3 a monoclonal antibody against CD40, CD40 L as defined by OMIM*300386 or C4BP as defined by OMIM*120830.

The term “in combination” encompasses any grouping or sub-grouping of ligands of the tumor necrosis superfamily as defined herein above with any grouping or sub-grouping of stimulators of CD40 molecules.

In a further particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 at least one polypeptide with human leukocyte interferon activity. In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules as defined herein above, at least one polypeptide with human leukocyte interferon activity.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules and at least one ligand of the tumor necrosis factor superfamily as defined herein above, at least one polypeptide with human leukocyte interferon activity.

The term “polypeptide with human leukocyte interferon activity” refers to a protein having antiviral and antioncogenic properties, showing macrophage and natural killer lymphocyte activation, as well as enhancement of major histocompatibility complex glycoprotein classes I and II, and being able to present foreign, e.g. microbial peptides to T cells, which is produced by human leukocytes. Preferably the term relates to interferon-α (IFN-α), more preferably to human interferon-α as defined by OMIM*147660.

In a more preferred embodiment of the present invention a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 human interferon-α as defined by OMIM*147660.

In another embodiment of the present invention, a pharmaceutical composition as defined herein above may be combined with a vaccine. The term “combined” means that the ingredients of a pharmaceutical composition as defined herein above may be mixed with a vaccine compound. Alternatively, the term also refers to an administration of a pharmaceutical composition as defined herein above together with a vaccine compound. The term “vaccine compound” as used herein above refers to any suitable vaccine or vaccination compound known to the skilled person. Preferably, it refers to (i) an inactivated vaccine, (ii) an attenuated vaccine, (iii) a subunit vaccine and (iv) a DNA vaccine.

The term “inactivated vaccine” means a vaccine or composition comprising virus particles which were grown in culture and subsequently killed or destroyed, preferably by using heat or formaldehyde. Such virus particles typically cannot replicate, but certain virus proteins, e.g. capsid proteins, are intact enough to be recognized by the immune system and evoke a response. The term “attenuated vaccine” means a vaccine or composition comprising live virus particles with a low virulence. Typically, live attenuated virus particles may reproduce, but very slowly. These vaccines may be produced by any suitable method known to the skilled person, normally by growing viruses in tissue cultures that will select for less virulent strains, or by mutagenesis or targeted deletions in genes required for virulence.

The term “subunit vaccine” means a vaccine or composition comprising an antigen, which is provided to the immune system without the introduction of viral particles, whole or otherwise. The term “antigen” refers to any antigenic determinant of a viral structure, e.g. peptide or proteins structures or surface structure of non-proteineous origin, e.g. sugar structures or trees. A subunit vaccine may be produced by any suitable method known to the person skilled in the art. Typically the production may involve the isolation of a specific protein or protein portion or of sugar structures from a virus and their administration as vaccine or vaccine composition.

The term “DNA vaccine” relates to DNA compositions created from an infectious agent's DNA or encoding an infectious agent's structural components, which is typically inserted into cells, e.g. human or animal cells, and expressed therein.

Cells of the immune system that recognize the proteins expressed may subsequently mount an attack against these proteins and cells expressing them.

Preferred are vaccines which have been approved by a competent authority, e.g. the EMEA or the FDA, or which can be derived from the red list of medicaments published in Germany.

In a further particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 at least one vaccine protein antigen.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules as defined herein above, at least one vaccine protein antigen.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules and at least one ligand of the tumor necrosis factor superfamily as defined herein above, at least one vaccine protein antigen.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules and at least one ligand of the tumor necrosis factor superfamily as defined herein above and at least one polypeptide with human leukocyte interferon activity, at least one vaccine protein antigen.

The term “vaccine protein antigen” refers to any protein antigen suitable for human or veterinary vaccination known to the person skilled in the art. Such antigens may be derived, for example, from a qualified vaccination textbook, e.g. from “Epidemiology and Prevention of Vaccine-Preventable Diseases”, The Pink Book: Course Textbook, 10^{th} ed, (2008) Atkinson et al. ed., Department of Health and Human Services, CDC.

Preferably, the term relates to vaccines including toxoids (e.g. inactivated bacterial toxin) or enzymatically inactive subunits of toxins, or inactive viral particles, or subvirion products, more preferably to vaccines as mentioned in Epidemiology and Prevention of Vaccine-Preventable Diseases”, The Pink Book: Course Textbook, 10^{th} ed, (2008) Atkinson et al. ed., Department of Health and Human Services, CDC.

In a more preferred embodiment of the present invention a pharmaceutical composition of the present invention may comprise in addition to IL-21 and/or an IL-21 variant as defined herein above and IL-2 and/or IL-4 and/or IGIP, Syntenin-1, Galectin-1 or Galectin-3, vaccine protein antigen diphtheria toxoid vaccine and/or tetanus toxoid diphtheria toxoid.

In a further particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 at least one vaccine polysaccharide antigen.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules as defined herein above, at least one vaccine polysaccharide antigen.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules and at least one ligand of the tumor necrosis factor superfamily as defined herein above, at least one vaccine polysaccharide antigen.

In another also particularly preferred embodiment, a pharmaceutical composition of the present invention may comprise in addition to IL-21 and/or an IL-21 variant as defined herein above and IL-2 and/or IL-4 and at least one stimulator of CD40 molecules and at least one ligand of the tumor necrosis factor superfamily as defined herein above and at least one polypeptide with human leukocyte interferon activity, at least one vaccine polysaccharide antigen.

In another also particularly preferred embodiment, a pharmaceutical composition of the present invention may comprise in addition to IL-21 and/or an IL-21 variant as defined herein above and IL-2 and/or IL-4 and/or IGIP, Syntenin-1, Galectin-1 or Galectin-3 and at least one stimulator of CD40 molecules and at least one ligand of the tumor necrosis factor superfamily as defined herein above and at least one polypeptide with human leukocyte interferon activity and at least one vaccine protein antigen, at least one vaccine polysaccharide antigen.

The term “vaccine polysaccharide antigen” refers to any polysaccharide antigen suitable for human or veterinary vaccination known to the person skilled in the art. Such antigens may be derived, for example, from a qualified vaccination textbook, e.g. from “Epidemiology and Prevention of Vaccine-Preventable Diseases”, The Pink Book: Course Textbook, 10^{th} ed, (2008) Atkinson et al. ed., Department of Health and Human Services, CDC. Preferably, the term relates to vaccines composed of pure cell wall polysaccharide from bacteria. Also preferred are conjugated polysaccharide vaccines, e.g. vaccines in which the polysaccharide is chemically linked to a protein in order to increase its potency. Corresponding methods are known to the person skilled in the art or can be derived from suitable textbooks, e.g. from “Epidemiology and Prevention of Vaccine-Preventable Diseases”, The Pink Book: Course Textbook, 10^{th} ed, (2008) Atkinson et al. ed., Department of Health and Human Services, CDC.

In a more preferred embodiment of the present invention a pharmaceutical composition of the present invention may comprise in addition to IL-21 and/or an IL-21 variant as defined herein above and IL-2 and/or IL-4 and/or IGIP, Syntenin-1, Galectin-1 or Galectin-3, a vaccine protein antigen preferably a pneumococcal vaccine and/or a heamophilus influenzae type B vaccine.

If present in a pharmaceutical composition the above mentioned factors may be present in any suitable ration known to the person skilled in the art, e.g. the factors may be present in equimolar amounts, or one factor may be present in a ratio of about 5:1 to 20:1 with respect to the other factor. These ratios may also be combinable with ratios between IL-21, IL-21 variants and IL-2 or IL-4 as well as ratios between IL-21 or IL-21 variants and IGIP, Syntenin-1, Galectin-1 or Galectin-3, as well as ratios between IL-2 or IL-4 and IGIP, Syntenin-1, Galectin-1 or Galectin-3 as defined herein above. The exact and/or optimal ratio may be adjusted by carrying out tests as known to the person skilled in the art, e.g. tests as described herein above in the context of IL-21 variant proteins.

The pharmaceutical composition of the present invention may be administered to a patient according to any suitable dosage regimen known to the person skilled in the art. The dosages may preferably be given once a week, more preferably 2 times, 3 times, 4 times, 5 times or 6 times a week and most preferably daily and or 2 times a day or more often. The present invention further envisages that the dosage may be given directly after an immunological reaction or after an infection involving the immune system. However, during progression of the treatment the dosages can be given in much longer time intervals and in need can be given in much shorter time intervals, e.g., several times a day. In a preferred case the immune response may be monitored using herein described methods and further methods known to those skilled in the art and dosages are optimized, e.g., in time, amount and/or composition. Progress can be monitored by periodic assessment. It is also envisaged that the pharmaceutical composition of the present invention is employed in co-therapy approaches, i.e. in co-administration with other medicaments or drugs, for example antibiotics, antiviral medicaments or IgG or IgA immunoglobulins for replacement therapies etc.

In a further aspect the present invention relates to a kit comprising IL-21 and/or an IL-21 variant as mentioned herein above and IGIP. In a further aspect the present invention relates to a kit comprising IL-21 and/or an IL-21 variant as mentioned herein above and Syntenin-1. In a further aspect the present invention relates to a kit comprising IL-21 and/or an IL-21 variant as mentioned herein above and Galectin-1. In a further aspect the present invention relates to a kit comprising IL-21 and/or an IL-21 variant as mentioned herein above and Galectin-3. In a further aspect the present invention relates to a kit comprising IL-21 and/or an IL-21 variant as mentioned herein above and IL-2 and/or IL-4 and IGIP. In a further aspect the present invention relates to a kit comprising IL-21 and/or an IL-21 variant as mentioned herein above and IL-2 and/or IL-4 and Syntenin-1. In a further aspect the present invention relates to a kit comprising IL-21 and/or an IL-21 variant as mentioned herein above and IL-2 and/or IL-4 and Galectin-1. In a further aspect the present invention relates to a kit comprising IL-21 and/or an IL-21 variant as mentioned herein above and IL-2 and/or IL-4 and Galectin-3. In a further aspect the present invention relates to a kit comprising IL-21 and/or an IL-21 variant as mentioned herein above and IL-2 and/or IL-4 and/or IGIP and/or Syntenin-1, and/or Galectin-1 and/or Galectin-3.

In a further aspect the present invention also provides a kit that can be used in the context of the administration of the pharmaceutical composition as defined herein above. In particular, a kit according to the present invention may be used for the treatment of a primary humoral immunodeficiency disease, more preferably a disease involving a reduction in the level of secreted IgG and/or IgA, even more preferably a disease connected with a symptom of impairment or blocking of immunoglobulin isotype-switching and most preferably a selective deficiency of IgA, a common variable immunodeficiency, a selective deficiency of IgG subclasses, an immunodeficiency with increased IgM, or an X-linked agammaglobulinaemia as defined herein above in the context of pharmaceutical compositions of the present invention.

In one preferred embodiment, a kit according to the present invention comprises IL-21 and IL-4. In another preferred embodiment a kit according to the present invention comprises an IL-21 variant as mentioned herein above and IL-4. In yet another preferred embodiment a kit according to the present invention comprises IL-21 and IL-2. In yet another preferred embodiment a kit according to the present invention comprises an IL-21 variant as mentioned herein above and IL-2. In another preferred embodiment a kit according to the present invention comprises IL-21 and an IL-21 variant as mentioned herein above and IL-2 or IL-4. In yet another preferred embodiment a kit according to the present invention comprises IL-21 and an IL-21 variant as mentioned herein above and IL-2 and IL-4. In another preferred embodiment a kit according to the present invention comprises IL-21 and/or an IL-21 variant as mentioned herein above and IL-4 and/or IL-2 and IGIP. In another preferred embodiment a kit according to the present invention comprises IL-21 and/or an IL-21 variant as mentioned herein above and IL-4 and/or IL-2 and Syntenin-1. In another preferred embodiment a kit according to the present invention comprises IL-21 and/or an IL-21 variant as mentioned herein above and IL-4 and/or IL-2 and Galectin-1. In another preferred embodiment a kit according to the present invention comprises IL-21 and/or an IL-21 variant as mentioned herein above and IL-4 and/or IL-2 and Syntenin-3. In another preferred embodiment a kit according to the present invention comprises IL-21 and/or an IL-21 variant as mentioned herein above and IL-4 and/or IL-2 and/or IGIP and/or Syntenin-1 and/or Galectin-1 and/or Galectin-3.

In a further preferred embodiment of the present invention the ratio between ingredients in the kit may be suitably adjusted according to the skilled person's knowledge. For instance the ratio between IL-21 and IL-4 in a kit may be adjusted to be between about 1:1 and 100:1, preferably between about 5:1 and 25:1, more preferably about 20:1. Alternatively, the ratio between an IL-21 variant as defined herein above and IL-4 in a kit may be adjusted to be between about 1:1 and 100:1, preferably between about 5:1 and 25:1, more preferably to be about 20:1.

In a further preferred embodiment the ratio between IL-21 and IL-2 in a kit may be adjusted to be between about 1:1 and 80:1, preferably between about 5:1 and 20:1, more preferably to be about 15:1. Alternatively, the ratio between an IL-21 variant as defined herein above and IL-2 in a kit may be adjusted to be between about 1:1 and 80:1, preferably between about 5:1 and 20:1, more preferably to be about 15:1.

In a further preferred embodiment the ratio between IL-21 and IGIP in a kit may be adjusted to be between about 1:1 and 80:1, preferably between about 5:1 and 20:1, more preferably to be about 15:1. Alternatively, the ratio between an IL-21 variant as defined herein above and IGIP in a kit may be adjusted to be between about 1:1 and 80:1, preferably between about 5:1 and 20:1, more preferably to be about 15:1.

In a further preferred embodiment the ratio between IL-21 and Syntenin-1 in a kit may be adjusted to be between about 1:1 and 80:1, preferably between about 5:1 and 20:1, more preferably to be about 15:1. Alternatively, the ratio between an IL-21 variant as defined herein above and Syntenin-1 in a kit may be adjusted to be between about 1:1 and 80:1, preferably between about 5:1 and 20:1, more preferably to be about 15:1.

In a further preferred embodiment the ratio between IL-21 and Galectin-1 in a kit may be adjusted to be between about 1:1 and 80:1, preferably between about 5:1 and 20:1, more preferably to be about 15:1. Alternatively, the ratio between an IL-21 variant as defined herein above and Galectin-1 in a kit may be adjusted to be between about 1:1 and 80:1, preferably between about 5:1 and 20:1, more preferably to be about 15:1.

In a further preferred embodiment the ratio between IL-21 and Galectin-3 in a kit may be adjusted to be between about 1:1 and 80:1, preferably between about 5:1 and 20:1, more preferably to be about 15:1. Alternatively, the ratio between an IL-21 variant as defined herein above and Galectin-3 in a kit may be adjusted to be between about 1:1 and 80:1, preferably between about 5:1 and 20:1, more preferably to be about 15:1.

The ingredients of a kit may, according to the present invention, be comprised in one or more containers or separate entities. They may preferably be formulated as pharmaceutical compositions or medicaments, more preferably they may be formulated as has been described herein above in the context of the pharmaceutical compositions of the present invention, e.g. they may comprise suitable pharmaceutical carriers etc. Particularly preferred are formulations for aersolic or inhalational administration as mentioned herein above in the context of pharmaceutical compositions of the invention. The kit according to the present invention may optionally also comprise a documentation which indicates the use or employment of the kit and its components. Preferably, instructions comprised in the kit of the present invention may comprise recommended treatment options, dosage regimens etc.

In a further preferred embodiment a kit according to the present invention may additionally comprise at least one of the elements:
(i) a stimulator of CD40 molecules;
(ii) a ligand of the tumor necrosis superfamily;
(iii) a polypeptide with human leukocyte interferon activity;
(iv) a vaccine protein antigen; and
(v) a vaccine polysaccharide antigen.

The terms “stimulator of CD40 molecules”, “ligand of the tumor necrosis superfamily”, “polypeptide with human leukocyte interferon activity”, “vaccine protein antigen” and “vaccine polysaccharide antigen” have been defined in the context of pharmaceutical compositions of the present invention. These definitions apply correspondingly also to kits of the present invention. Also preferred are in the context of kits of the present invention all members of these groups, as well as all potential combinations thereof, as defined herein above. A kit may in specific embodiments of the present invention comprise any group or subgroup of elements (i) to (v) together with IL-21 and/or IL-21 variants as defined herein above, IL-2 and/or IL-4, IGIP, Syntenin-1, Galectin-1 or Galectin-3. Preferably a kit according to the present invention comprises at least element (i), i.e. a stimulator of CD40 molecules together with IL-21 and/or IL-21 variants as defined herein above, IL-2 and/or IL-4 and/or IGIP, Syntenin-1, galection-1 or Galectin-3.

In a more preferred embodiment of the present invention a kit of the present invention may comprise in addition to IL-21 and/or an IL-21 variant as defined herein above and IL-2 and/or IL-4 and/or IGIP, Syntenin-1, Galectin-1 or Galectin-3, an antibody, preferably a monoclonal antibody, against CD40, CD40 L, preferably as defined by OMIM*300386 or C4BP, preferably as defined by OMIM*120830.

In a further particularly preferred embodiment of the present invention a kit of the present invention may comprise in addition to IL-21 and/or an IL-21 variant as defined herein above and IL-2 and/or IL-4 and/or IGIP, Syntenin-1, Galectin-1 or Galectin-3, BAFF, preferably as defined by OMIM*603969, or LIGHT, preferably as defined by OMIM*604520.

In a further particularly preferred embodiment of the present invention a kit of the present invention may comprise in addition to IL-21 and/or an IL-21 variant as defined herein above and IL-2 and/or IL-4 and/or IGIP, Syntenin-1, Galectin-1 or Galectin-3, interferon-α, preferably human interferon-α, more preferably human interferon-α as defined by OMIM*147660.

In a further particularly preferred embodiment of the present invention a kit of the present invention may comprise in addition to IL-21 and/or an IL-21 variant as defined herein above and IL-2 and/or IL-4 and/or IGIP, Syntenin-1, Galectin-1 or Galectin-3, a vaccine protein antigen, preferably a diphtheria toxoid vaccine and/or a tetanus toxoid diphtheria toxoid.

In a further particularly preferred embodiment of the present invention a kit of the present invention may comprise in addition to IL-21 and/or an IL-21 variant as defined herein above and IL-2 and/or IL-4 and/or IGIP, Syntenin-1, Galectin-1 or Galectin-3, a vaccine protein antigen, preferably a pneumococcal vaccine and/or a heamophilus influenzae type B vaccine.

In a further embodiment a kit may also comprise any other suitable vaccine as known to the person skilled in the art, e.g. to an inactivated vaccine composition as defined herein above, an attenuated vaccine composition as defined herein above, or a DNA vaccine composition as defined herein above.

The present invention relates in a further embodiment to a kit as defined herein above, wherein the interim between the administration of the ingredients IL-2 or IL-4, or IL-2 and IL-4 and/or IGIP, Syntenin-1, Galectin-1 or Galectin-3 and IL-21 or an IL-21 variant as defined herein above, or IL-21 and an IL-21 variant as defined herein above is between about 1 minute and 12 hours. The term “interim” refers to the period of time between the administration of either of the two groups IL-21 and/or IL-21 variant on the one hand and IL-2 and/or IL-4 and/or IGIP, Syntenin-1, Galectin-1 or Galectin-3 on the other hand. The scheme thus encompasses situations in which the group of IL-21 and/or IL-21 variants as define herein above is administered first and subsequently, after the period of time as indicated above has passed, the second group of IL-2 and/or IL-4 and/or IGIP, Syntenin-1, Galectin-1 or Galectin-3 is administered to a patient, as well as situations in which the groups of IL-2 and/or IL-4 and/or IGIP, Syntenin-1, Galectin-1 or Galectin-3 is administered first and subsequently, after the period of time as indicated above has passed, the second group of IL-21 and/or IL-21 variants as defined herein above is administered to a patient. The interim may preferably be between about 1 minute and 6 hours, or between about 5 minutes and 3 hours, or between about 10 minutes and 2 hours, or between about 15 minutes and 1 hour, or between about 20 minutes and 50 minutes, or about 25 minutes, 30 minutes, 35 minutes, 40 minutes or 45 minutes. The interim as defined herein above may be adjusted according to the concrete situation, e.g. the patient's physiological situation, dosage deliberations, parameters derivable form parallel treatment processes etc. In a particularly preferred embodiment the interim between the first administration of IL-21 and/or IL-21 variants as defined herein above and the subsequent administration of IL-2 and/or IL-4 and/or IGIP, Syntenin-1, Galectin-1 or Galectin-3 may be 12 hours.

In a further embodiment, the time interval to be used may be indicated in the instructions provided with a kit according to the present invention.

The present invention relates in a further embodiment to a kit as defined herein above, wherein the interim between the administration of the ingredients IL-2 or IL-4, or IL-2 and IL-4 plus IL-21 or an IL-21 variant as defined herein above and/or IGIP, Syntenin-1, Galectin-1 or Galectin-3, or plus IL-21 and an IL-21 variant as defined herein above and/or IGIP, Syntenin-1, Galectin-1 or Galectin-3 on the one hand (group 1) and at least one of the elements (group 2):
(i) a stimulator of CD40 molecules (group 2a);
(ii) a ligand of the tumor necrosis superfamily (group 2b);
(iii) a polypeptide with human leukocyte interferon activity (group 2c);
(iv) a vaccine protein antigen (group 2d); and
(v) a vaccine polysaccharide antigen (group 2e)
   on the other hand is between about 12 hours and 72 hours. Preferred ingredients of groups 2a to 2e are those indicated herein above.

The interim between group 1 and any of the subgroups of group 2, i.e. groups 2a, b, c, d or e may be different within the indicated interim of about 12 hours to 72 hours, depending on the exact combination of elements. Preferably, the interim between the administration of group 1 and group 2a may be between about 0 hours and 24 hours, more preferably between about 3 and 24 hours, the interim between the administration of group 1 and group 2b may be between about 24 hours and 72 hours, the interim between the administration of group 1 and group 2c may be between about 0 and 24 hours, more preferably between about 3 and 24 hours, the interim between the administration of group 1 and group 2d may be between about 12 hours and 72 hours, the interim between the administration of group 1 and group 2e may be between about 12 hours and 72 hours

In case more than one group of subgroups 2a to 2e may be administered there may additionally be further interims between the administration of these additional sub-groups, preferably up to a total interim between the first compound to be administered and the last compound to be administered of 72 hours.

The interim between group 1 and any of the subgroups of group 2, i.e. groups 2a, b, c, d, e may preferably be between about 12 hours and 36 hours, or between about 24 hours and 48 hours, or between about 36 hours and 72 hours, or between about 12 hours and 48 hours, or between about 24 hours and 72 hours, or between about 24 hours and 36 hours, or between about 36 hours and 48 hours, or between 48 hours and 72 hours.

The interim as defined herein above may be adjusted according to the concrete situation, e.g. the patient's physiological situation, dosage deliberations, parameters derivable form parallel treatment processes etc.

In a particularly preferred embodiment the interim between the first administration of group 1 and any of the sub-groups of group 2, i.e. groups 2a, b, c, d, e may be 72 hours.

In a further embodiment, the time interval to be used for the administration of groups 1 and 2a, b, c, d, e may be indicated in the instructions provided with a kit according to the present invention.

The kit of the present invention may be administered to a patient according to any suitable dosage regimen known to the person skilled in the art. The kit or kit components may preferably be given once a week, more preferably 2 times, 3 times, 4 times, 5 times or 6 times a week and most preferably daily and or 2 times a day or more often, unless otherwise indicated, e.g. via the provision of interim times as defined herein above The present invention further envisages that the dosage may be given directly after an immunological reaction or after an infection involving the immune system. However, during progression of the treatment the dosages may be given in much longer time intervals and in need can be given in much shorter time intervals, e.g., several times a day. In a preferred case the immune response may be monitored using herein described methods and further methods known to those skilled in the art and dosages are optimized, e.g., in time, amount and/or composition. Progress can be monitored by periodic assessment. It is also envisaged that the kit is employed in co-therapy approaches, i.e. in co-administration with other medicaments or drugs, for example antibiotics, antiviral medicaments or IgG or IgA immunoglobulins for replacement therapies etc.

In a particularly preferred embodiment of the present invention the components of the pharmaceutical composition or kit of the present invention, i.e. IL-21 and/or the IL-21 variant as defined herein above, and IL-2 and/or IL-4 and/or IGIP, Syntenin-1, Galectin-1 or Galectin-3, and optionally a stimulator of CD40 molecules, preferably an anti-CD40 antibody, a CD40 ligand or C4BP; a ligand of the tumor necrosis superfamily, preferably BAFF or LIGHT; a polypeptide with human leukocyte interferon activity, preferably interferon-α; a vaccine protein antigen, preferably a diphtheria toxoid vaccine and/or a tetanus toxoid vaccine; and/or a vaccine polysaccharide antigen, preferably a pneumococcal vaccine and/or a heamophilus influenzae type B vaccine may be provided in the form of a living therapeutic. The term “living therapeutic” means that said components of the pharmaceutical composition or kit of the present invention as mentioned above is expressed in any suitable live carrier.

The present invention thus relates in a further aspect to a live carrier expressing IL-21 or an IL-21 variant as mentioned herein above and at least one element selected from the group consisting of IL-4, IL-2, IGIP, Syntenin-1, Galectin-1 and Galectin-3. In a preferred embodiment said live carrier may additionally also express one, two three or four elements in any combination selected from:
(i) a stimulator of CD40 molecules, preferably an anti-CD40 antibody, a CD40 ligand (CD40L) or C4BP;
(ii) a ligand of the tumor necrosis superfamily, preferably BAFF or LIGHT;
(iii) a polypeptide with human leukocyte interferon activity, preferably Interferon-α (IFN-α); and
(iv) a vaccine protein antigen.

Accordingly, the present invention relates to polynucleotides encoding components of the pharmaceutical composition or kit of the present invention as mentioned above which are suitable for expression in a living cell or carrier, e.g. the polynucleotide of SEQ ID NO: 13, 14 or 15, or a polynucleotide encoding the polypeptide of SEQ ID NO: 1, 2, 3, 4, 5, 6, 7, 8, 10, 11 or 12. Accordingly, the present invention also relates to vectors containing the polynucleotides of the present invention, appropriate host cells, and the production of polypeptides by recombinant techniques in said host cells. The vector may be, for example, a phage, plasmid, viral, or retroviral vector. The polynucleotides encoding components of the pharmaceutical composition or kit of the present invention as mentioned above may be joined to a vector containing a selectable marker for propagation in an appropriate host. Preferably, the polynucleotide insert should be operatively linked to an appropriate promoter as mentioned herein above.

Such a vector may comprise one, two, three or more genetic units encoding said components. Accordingly, a living therapeutic or live carrier may comprise one, two, three or more vectors at the same time. There coexistence may be regulated according to suitable means and methods known to the person skilled in the art.

The expression of said components may take place on the surface of the living therapeutic or live carrier, leading to an established, sustained or prolonged presentation of the expressed proteins or peptides, or may alternatively be in the form of a secretion of proteins or peptides from the cell(s), leading to an increase of the concentration of the proteins or peptides in the extracellular environment. For both scenarios suitable vectors, gene cassettes, e.g. comprising secretion signals etc., as known to the person skilled in the art may be used.

The term “live carrier” relates to any appropriate living host cell or virus known to the person skilled in the art. Representative examples of appropriate hosts include, but are not limited to, bacterial cells such as Escherichia coli or Lactobacillus, fungal cells, such as yeast cells, protozoa, insect cells, or animal cells. Preferably, the term relates to attenuated bacteria, attenuated fungal cells or attenuated protozoa. Representative examples of appropriate viruses include viruses of the group of adenoviruses, retrovirues or lentirviruses, preferably attenuated viruses of the group of adenoviruses, retroviruses or lentirviruses.

In a preferred embodiment, probiotic bacterial cells, in particular probiotic Escherichia coli or Lactobacillus cells, may be used. More preferably, cells of Escherichia coli Nissle 1973 and even more preferably cells of Lactobacillus casei or Lactobacillus zeae 393 may be used. Such bacterial cells, in particular the Lactobacillus cells, may be used for the gastrointestinal production of IL-21 or an IL-21 variant as mentioned herein above, IL-4, IL-2, IGIP, Syntenin-1, Galectin-1 and Galectin-3, as well as immunoglobulines, preferably for the presentation of IL-21 or an IL-21 variant together with IL-2 and/or IL-4 and CD40L molecules or the reconstitution of the gastrointestinal production of immunoglobulines. The bacterial cells may be administered to a patient in any suitable form known to the person skilled in the art, preferably orally.

Accordingly, the cells may be cultured ex vivo, e.g. under laboratory conditions. Appropriate culture media and conditions for the above-described host cells are known in the art. Subsequently the cells may be transformed with suitable expression vectors expressing one or more of the components of the pharmaceutical composition or kit of the present invention, i.e. IL-21 and/or the IL-21 variant as defined herein above, and IL-2 and/or IL-4, and/or IGIP, Syntenin-1, Galectin-1 and Galectin-3, a stimulator of CD40 molecules, preferably an anti-CD40 antibody, a CD40 ligand or C4BP; a ligand of the tumor necrosis superfamily, preferably BAFF or LIGHT; a polypeptide with human leukocyte interferon activity, preferably interferon-α; a vaccine protein antigen, preferably a diphtheria toxoid and/or a tetanus toxoid; and/or a vaccine polysaccharide antigen, preferably a pneumococcal cell wall polysaccharide and/or a heamophilus influenzae type B vaccine. The expression of these components may be carried out in a single cell or cell type, e.g. by expressing the compounds from single vector, or from different vectors, e.g. based on the use of two or more different selection marker genes. Alternatively, the expression of these components may be carried out in more than one cell or cell type, e.g. by expressing only between one and three compounds in one cell or cells type. Via the use of different expression vectors and/or distinguishable selection markers all components as mentioned above may be transferred, stabilized and expressed in the mentioned cell types.

Preferably, expression constructs or cassettes may be integrated into the genome of the organisms, more preferably without leaving selection marker or marker traces behind. Suitable methods for such integration procedures are known to the person skilled in the art.

Among vectors preferred for use in bacteria are pQE70, pQE60 and pQE9, available from QIAGEN, Inc.; pBluescript vectors, Phagescript vectors, pNH8A, pNH16a, pNH18A, pNH46A, available from Stratagene Cloning Systems, Inc.; pKK223-3, pKK233-3, pDR540, pRIT5 available from Pharmacia Biotech, Inc., and pET vectors from Novagen. Among preferred eukaryotic vectors are pWLNEO, pSV2CAT, pOG44, pXTI and pSG available from Stratagene; and pSVK3, pBPV, pMSG and pSVL available from Pharmacia. Particularly preferred expression vectors for use in Lactobacillus systems may include pGhost4, pGhost5 and pGhost6 available from Appligene-Oncor, Illkirsh, France. Further, pIAb8, pIAV7, pPSC22, pH2515, pLP3537 and pUCL287 may be used. Also particularly preferred are vectors pLP402 and pTUAT. Other suitable vectors will be known to the skilled person. Correspondingly prepared cells may subsequently be administered to a patient in any suitable form known to the person skilled in the art., preferably in the form of a composition comprising a living therapeutic as defined herein above. More preferably, such a composition may comprise a microorganism, e.g. a Lactobacillus as described above in an amount between 10² to 10¹² cells, preferably 10³ to 10⁸ cells per mg in a solid form of the composition. In case of a liquid form of compositions, the amount of the microorganisms is between 10² to 10¹³ cells per ml. However, for specific compositions the amount of the microorganism may be different and/or adjusted according to suitable parameters known to the person skilled in the art.

In a particularly preferred embodiment a live carrier according to the present invention may be used for the treatment of a primary humoral immunodeficiency disease, more preferably for the treatment of a disease involving a reduction in the level of secreted IgG and/or IgA antibodies, even more preferably for the treatment of selective deficiency of IgA (IgAD), common variable immunodeficiency (CVID), selective deficiency of IgG subclasses (IgGsD), immunodeficiency with increased IgM (hyper-IgM-syndrome) or X-linked agammaglobulinaemia.

A composition comprising a living therapeutic or live carrier as defined herein above may preferably be used for a local administration, e.g. via the oral administration and in situ expression of the compound in the gastrointestinal zone. Alternatively, a living therapeutic or live carrier as defined herein above may be applied in the form of enemas, or by a direct administration to rectal parts of the gastrointestinal tract. Alternatively, a composition comprising a living therapeutic or live carrier as defined herein above may be used systemically, e.g. via different routes of administration. Examples may be an intravenous injection or a topical administration to certain organs like the mucosa in small intestine epithelium. The cells may subsequently stimulate resident B-cells leading to a production of immunoglobulines and a concomitant systemic secretion into interluminal spaces.

### EXAMPLES

### Example 1

### IL-21 Cooperates with IL-2 and IL-4 to Induce Immunoglobulin Production in Human B Cells

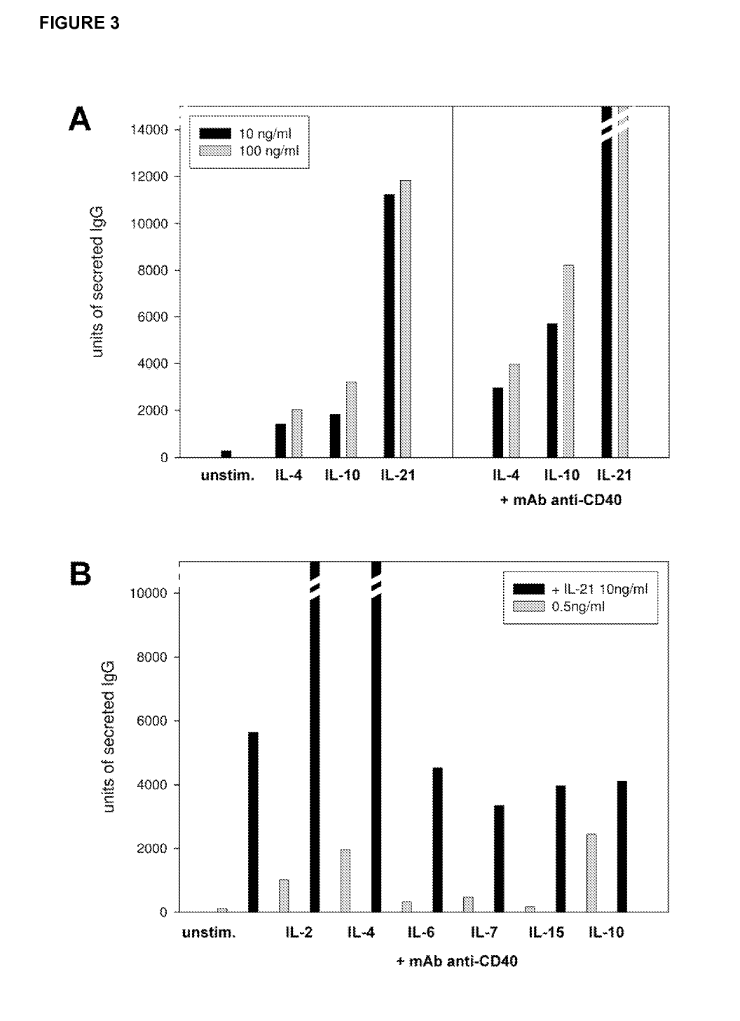

The efficacy of IL-21 was compared with the efficacy of IL-4 and IL-10 with regard to their potency to induce IgG and IgA production in anti-CD40 stimulated B cells within peripheral blood mononuclear cells (PBMC) from healthy donors (see, for example, FIG. 3A). It could, in particular, be shown that the Ig production, induced by IL-21, can be potentiated by other members of the γc family of cytokines (see FIG. 3B).

Cell Separation and Culture Conditions

To this end heparinised peripheral venous blood was obtained from randomly selected healthy donors. PBMC were isolated by Ficoll separation and stored in liquid nitrogen until use, according to a method described by Kreher C R et al. (2003) J Immunol Methods 278 (1-2) 79-93. Subsequently, 1×10⁶/ml PBMC were cultured for 5 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, Interleukin-10 or Interleukin-21 was supplemented in a range of concentrations of between 1 ng/ml and 100 ng/ml and Interleukin-2, Interleukin-4, Interleukin-6, Interleukin-7 and Interleukin-15 in a in a range of concentrations of between 0.1 ng/ml and 10 ng/ml (all from ImmunoTools, Friesoythe, Germany). If used, anti-human CD40 monoclonal antibody (clone S2C6) was added at 2 μg/ml (Mabtech AB, Stockholm, Sweden). On day 5 of culture, PBMC were washed with twice their culture volume as described above.

ELISPOT Assay and Determination of Immunoglobulin Amount

In order to determine the amount of immunoglobulines, an ELISPOT assay was performed. In particular, MultiScreenHTS Filter Plates (Millipore Corp., Bedford, Mass., USA) were pre-wet with 30% ethanol, rinsed three times with sterile PBS (sPBS) and coated overnight at 4° C. with either polyclonal rabbit anti-human IgG or IgA capture antibody (DAKO) diluted in sPBS at 10 μg/ml. After washing, plates were blocked for 3 hrs with sPBS containing 1% bovine serum albumin (SIGMA, St. Louis, Mo., USA). Cultured PBMC were plated at 1×10⁵ PBMC/well in cell culture medium as described above and incubated at 37° C. for 20 hrs in the presence of 5% CO₂. Thereafter, the plates were washed six times using PBS containing 0.01% Tween20 (PBS-Tween, SIGMA). Detection antibodies goat anti-human IgG-ALP (Mabtech AB) and goat anti-human IgA-ALP (SouthernBiotech) were diluted in PBS containing 0.5% bovine serum albumin and added at a final concentration of 2 μg/ml. After overnight incubation at 4° C., the plates were washed six times with PBS-Tween. Spot development was carried out using the BCIP/NBT Liquid Substrate System (SIGMA). ELISPOT plate analysis and subsequent enumeration of cell counts and immunoglobulin amount was performed on the AID EliSpot 04 HR Reader using appropriate AID reader software, release 4.0 (Autoimmun Diagnostika GmbH, Strassberg, Germany). The immunoglobulin amount measured according to this procedure can be derived from FIGS. 3A and 3B. In particular, the immunoglobulin amount shown in FIGS. 3A and B has been measured in a virtual unit that is equivalent to the surface in (0.01 mm)² multiplied by the intensity of a particular spot.

IL-21 stimulation alone gave rise to higher amounts of IgG and IgA than cultures stimulated with IL-4 or IL-10 (see FIG. 3A, similar data for IgA production are not shown). When B cells were activated by anti-CD40 stimulation, additional IL-21 stimulation resulted in much higher levels of IgG and IgA. In comparison with IL-4 or IL-10 induced Ig production of anti-CD40 stimulated PBMC, the production of IgG and IgA induced by IL-21 was several-fold higher (see FIG. 3A, similar data for IgA production are not shown). In contrast to IL-6, IL-7, IL-15, and IL-10, both IL-2 and IL-4 strongly enhanced IL-21 driven IgG and IgA production best, when added in a concentration of 0.5 ng/ml (see FIG. 3B, similar data for IgA production are not shown).

### Example 2

### IL-21 Promotes the Differentiation of Human B Cells into IgG or IgA Producing Plasma Cells

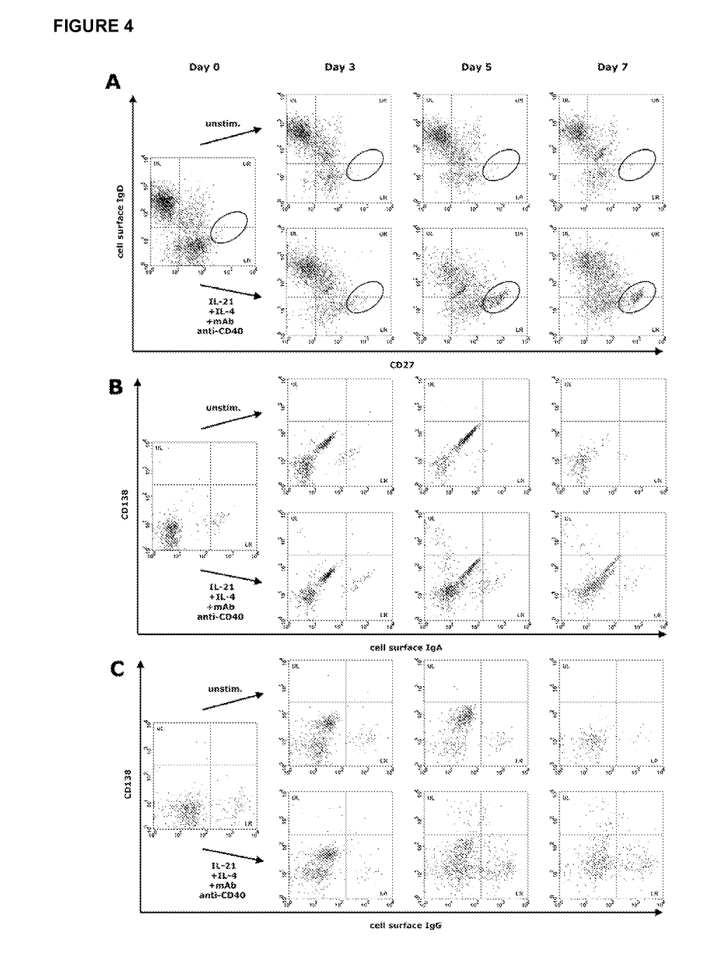

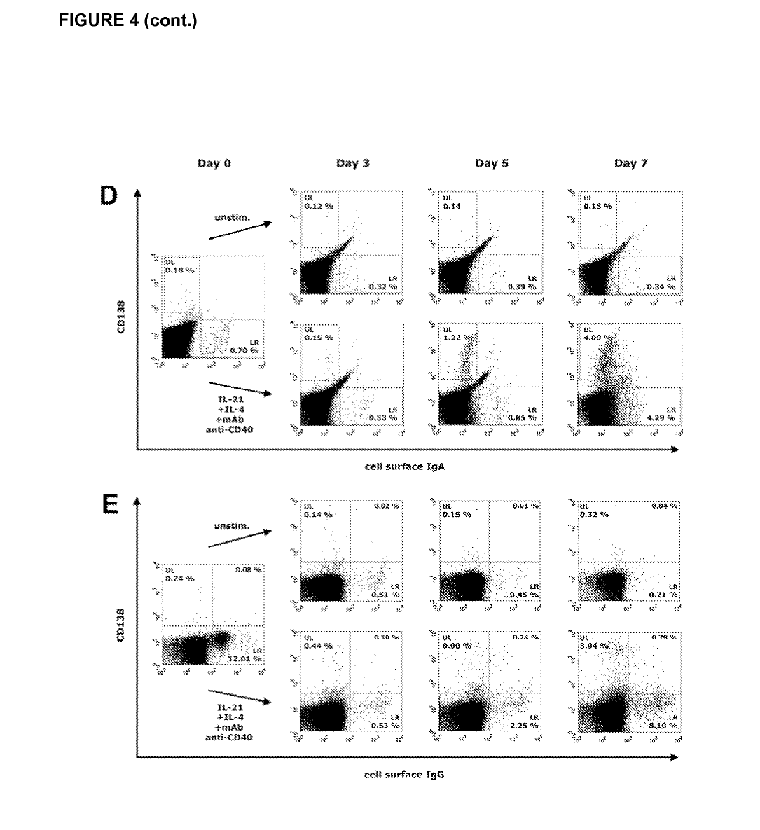

In the following experiment it could be shown that IL-21 has effects on CSR and Ig production and is a powerful promoter of B cell proliferation and plasma cell differentiation. In particular, it could be shown that a combination of IL-21, IL-4 and anti-CD40 mAb results in the formation of a CD27^{high} IgD⁻ CD38^{low} population within stimulated PBMC (see, for example, FIG. 4A). CD27^{high} IgD⁻ CD38^{low} cells represent activated B cells that are most likely committed to the plasma cell lineage. Further, two populations of Ig-secreting cells, consisting of sIg⁺ CD138⁻ and CD138⁺ sIg⁻ B cells, emerged during culture with IL-21, IL-4 and anti-CD40 mAb (see FIGS. 4B, C, D and E).

Cell Separation and Culture Conditions

To this end heparinised peripheral venous blood was obtained from randomly selected healthy donors. PBMC were isolated by Ficoll separation, according to a method described by Kreher C R et al. (2003) J Immunol Methods 278 (1-2) 79-93. Subsequently, 1×10⁶/ml PBMC were cultured for 7 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, Interleukin-21 was supplemented in a final concentrations of 10 ng/ml and Interleukin-4 in a final concentration of 0.5 ng/ml (both from ImmunoTools, Friesoythe, Germany), as established previously optimal for stimulation of PBMC. Anti-human CD40 monoclonal antibody (clone S2C6) was added at 2 μg/ml (Mabtech AB, Stockholm, Sweden). At days 0, 3, 5, and 7 of culture PBMC were subjected to flow cytometry analysis.

Flow Cytometry Analysis (FCM) and Determination of Cell Subsets

In order to determine the cell subsets, single-cell suspensions of cultured PBMC were distributed equally into three 12×75 mm round bottom tubes and washed with 5 ml of phosphate buffered saline (PBS) at 250×g for 5 minutes at room temperature. Subsequently, the following three cocktails of monoclonal antibodies were added each to a single tube to determine B cell and plasma cell populations: (1), anti-CD19-PC7, anti-CD27-FITC (both from DAKO, Glostrup, Denmark), anti-CD5-APC (BD Biosciences) and anti-IgD-PE (SouthernBiotech, Birmingham, Ala., USA). (2), anti-CD19-PC7, anti-IgD-PE, anti-CD38-APC and anti-CD77-FITC (both from BD Biosciences). (3), anti-CD19-PC7, anti-CD138-PE (Miltenyi Biotec, Gladbach, Germany), anti-IgA-FITC and anti-IgG-APC (both from Jackson ImmunoResearch, West Grove, Pa., USA) or anti-CD38-APC. Subsequently, single-cell suspensions were incubated with the cocktails of FACS antibodies for 30 minutes in the dark on ice. Following another washing step with 3 ml of PBS, the remaining cell pellet was resolved in 250 μof PBS containing 1% formaldehyde and immunophenotyping of lymphocytes was performed by four-colour cytometry on a FACSCalibur (BD Biosciences, Franklin Lakes, N.J., USA) using the CellQuest software (BD Biosciences).

FIG. 4 shows the expression of CD27, CD138 and surface IgD, IgG and IgA on CD19⁺ lymphogated cells in a representative healthy individual. Cell surface expression of these markers is represented on a four-decade log scale as dot plots of correlated x-axis and y-axis fluorescence. FCM analysis was performed at days 0, 3, 5 and 7 with PBMC cultured in the presence of IL-21 [10 ng/ml], IL-4 [0.5 ng/ml] and anti-human CD40 mAb [2 μg/ml]. In FIG. 4A quadrant markers were positioned to include naïve mature B cells (UL), natural effector B cells (UR), and IgD⁻ memory B cells (LR). The circle tags a population of CD27^{high} IgD⁻ B cells. In FIGS. 4B and D quadrant markers were positioned to separate CD138high plasma cells (UL) from sIgA⁺ B cells (LR). In FIGS. 4C and E quadrant markers were positioned to separate CD138^{high} plasma cells (UL) from sIgG^{high} B cells (LR).

### Example 3

### IL-21 Restores IgG and IgA Production in Patients with CVID and IgAD

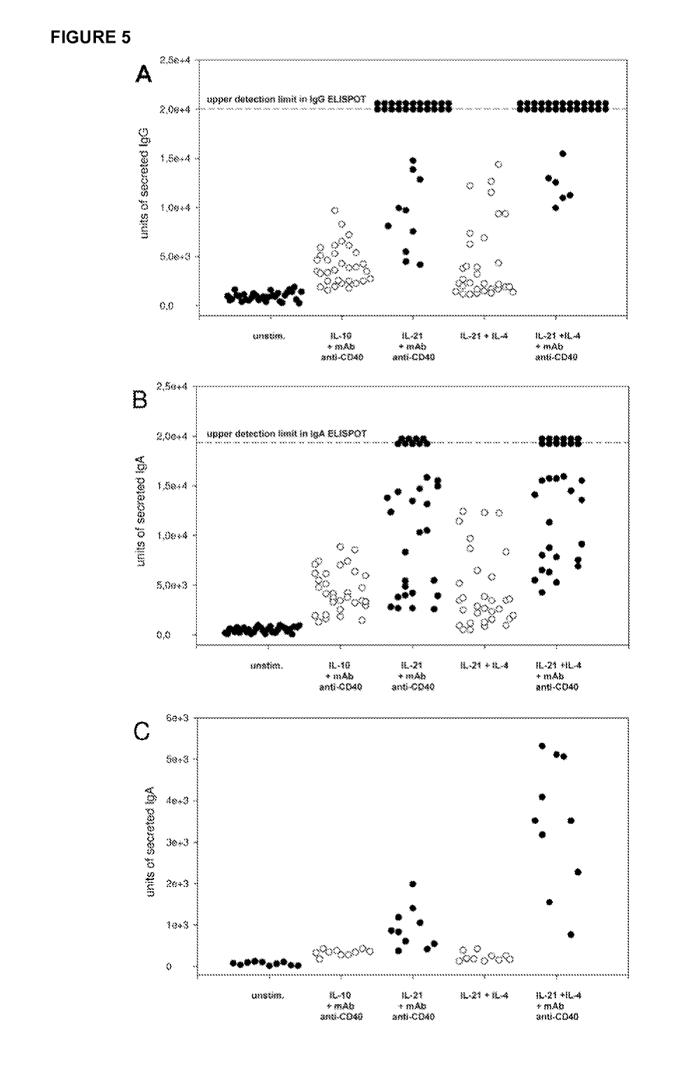

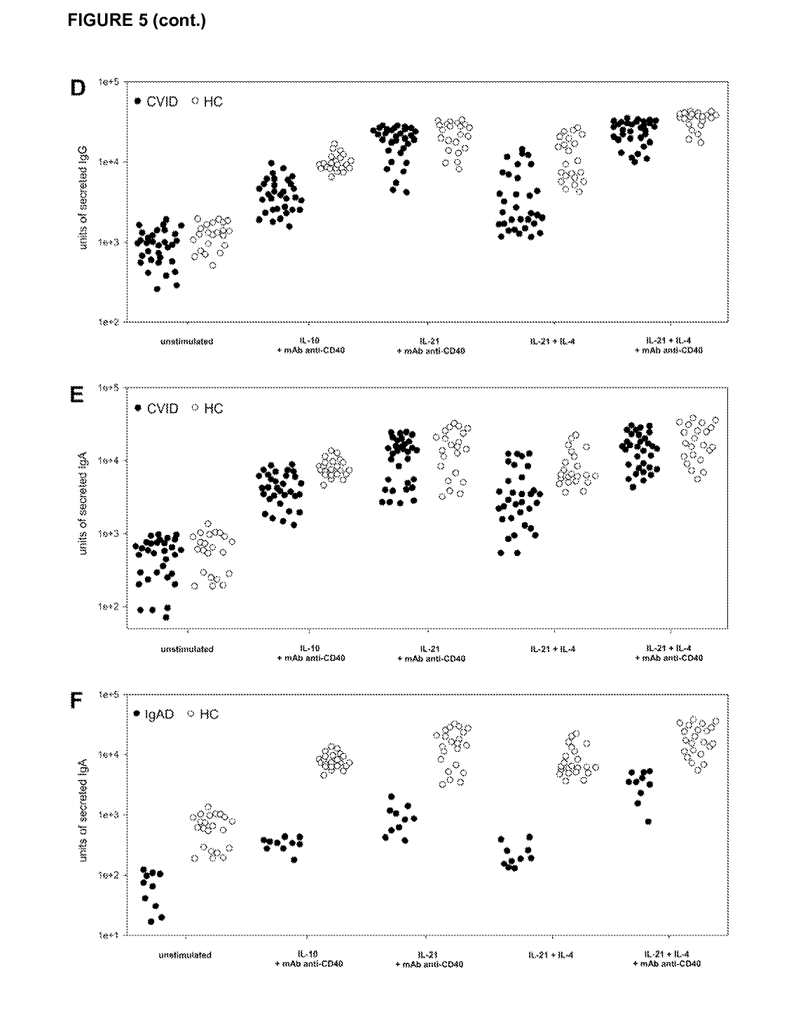

In the following experiment it could be shown that IL-21 is effective in restoration of the immunoglobulin production in vitro in primary immunodeficiency diseases, in particular in the both most prevalent PID diseases—common variable immunodeficiency (CVID) and selective IgA deficiency (IgAD), (see, for example, FIGS. 5A to F).

Cell Separation and Culture Conditions

To this end heparinised peripheral venous blood was obtained from patients with an established diagnosis of CVID or IgAD, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation and stored in liquid nitrogen until use, according to a method described by Kreher C R et al. (2003) J Immunol Methods 278 (1-2) 79-93. Subsequently, 1×10⁶/ml PBMC were cultured for 5 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, Interleukin-10 and Interleukin-21 were supplemented in a final concentrations of 10 ng/ml and Interleukin-4 in a final concentration of 0.5 ng/ml (both from ImmunoTools, Friesoythe, Germany), as established previously optimal for stimulation of PBMC. If used, anti-human CD40 monoclonal antibody (clone S2C6) was added at 2 μg/ml (Mabtech AB, Stockholm, Sweden). On day 5 of culture, PBMC were washed with twice their culture volume as described above.

ELISPOT Assay and Determination of Immunoglobulin Amount

In order to determine the amount of immunoglobulines, an ELISPOT assay was performed. In particular, MultiScreenHTS Filter Plates (Millipore Corp., Bedford, Mass., USA) were pre-wet with 30% ethanol, rinsed three times with sterile PBS (sPBS) and coated overnight at 4° C. with either polyclonal rabbit anti-human IgG or IgA capture antibody (DAKO) diluted in sPBS at 10 μg/ml. After washing, plates were blocked for 3 hrs with sPBS containing 1% bovine serum albumin (SIGMA, St. Louis, Mo., USA). Cultured PBMC were plated at 1×10⁵ PBMC/well in cell culture medium as described above and incubated at 37° C. for 20 hrs in the presence of 5% CO₂. Thereafter, the plates were washed six times using PBS containing 0.01% Tween20 (PBS-Tween, SIGMA). Detection antibodies goat anti-human IgG-ALP (Mabtech AB) and goat anti-human IgA-ALP (SouthernBiotech) were diluted in PBS containing 0.5% bovine serum albumin and added at a final concentration of 2 μg/ml. After overnight incubation at 4° C., the plates were washed six times with PBS-Tween. Spot development was carried out using the BCIP/NBT Liquid Substrate System (SIGMA). ELISPOT plate analysis and subsequent enumeration of cell counts and immunoglobulin amount was performed on the AID EliSpot 04 HR Reader using appropriate AID reader software, release 4.0 (Autoimmun Diagnostika GmbH, Strassberg, Germany). The immunoglobulin amount in FIGS. 5A, B and C is measured in a virtual unit that is equivalent to the surface in (0.01 mm)² multiplied by the intensity of a particular spot.

While IL-10 induced moderate Ig production in CVID (see FIGS. 5A, B, D and E) and IgAD patients (see FIGS. 5C and F), IL-21 stimulation resulted in several-fold higher amounts of secreted IgG and IgA (see FIGS. 5A-F). Even IL-21 plus IL-4 without further CD40 stimulation was more effective than IL-10 plus mAb anti-CD40 in inducing Ig production in some individuals. When Ig secretion in anti-CD40-activated PBMC was compared between cells stimulated solely with IL-21 or IL-21 plus IL-4, the latter markedly increased average IgG and IgA production almost to the levels observed in healthy donors.

### Example 4

### IL-21 Induces Active CSR in Individuals with CVID or IgAD

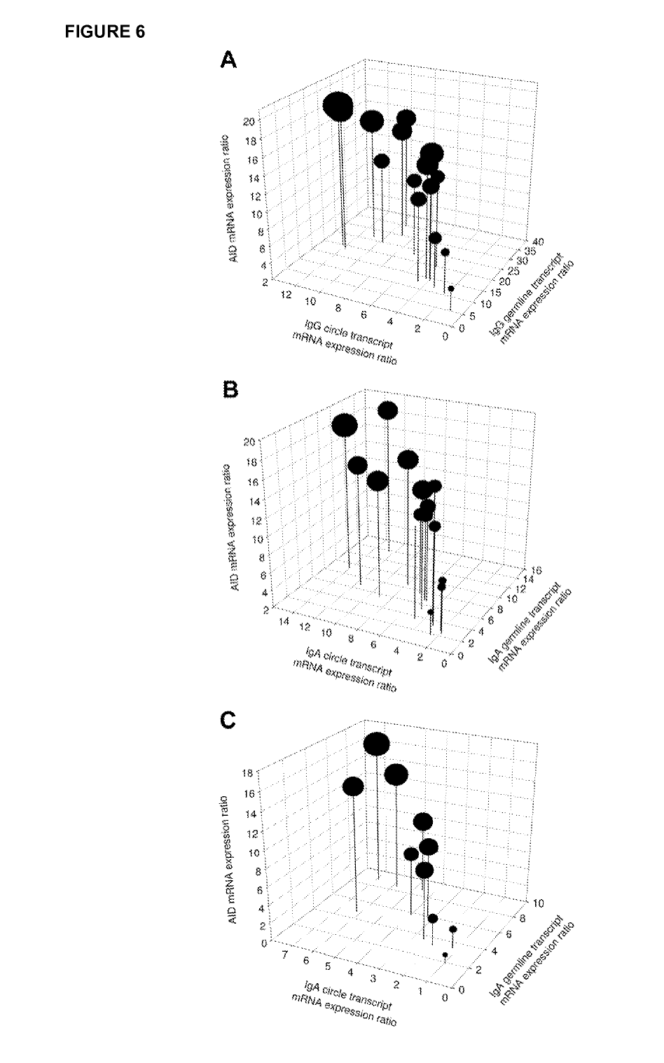

To elucidate the molecular mechanism involved in immunoglobulin production induced by IL-21 plus IL-4 and mAb anti-CD40 stimulation, mRNA levels were analysed for activation-induced cytidine deaminase (AID), Iγ-Cγ or Iα-Cα germline transcription (GLT) and Iγ-Cμ or Iα-Cμ switch circle transcripts (CT) in patients with CVID (see FIGS. 6A and B) or IgAD (see FIG. 6C).

Cell Separation and Culture Conditions

To this end heparinised peripheral venous blood was obtained from patients with an established diagnosis of CVID or IgAD, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation and stored in liquid nitrogen until use, according to a method described by Kreher C R et al. (2003) J Immunol Methods 278 (1-2) 79-93. Subsequently, 1×106/ml PBMC were cultured for 72 hrs in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, Interleukin-21 was supplemented in a final concentrations of 10 ng/ml and Interleukin-4 in a final concentration of 0.5 ng/ml (both from ImmunoTools, Friesoythe, Germany), as established previously optimal for stimulation of PBMC. Anti-human CD40 monoclonal antibody (clone S2C6) was added at 2 μg/ml (Mabtech AB, Stockholm, Sweden).

RNA Isolation and Real-Time Quantitative RT-PCR

Following 72 hrs of culture, RNA was extracted from all PBMC using RNeasy Plus Mini Kits (Qiagen, Hilden, Germany). One-step cDNA reverse-transcription and real-time PCR was conducted using the SYBR Green I RNA Master Mix (Roche Applied Science, Mannheim, Germany) and run on a LightCycler 2.0 System (Roche Applied Science). Sequence specific primers for the detection of β-actin, AID expression, as well as Iγ-Cγ or Iα-Cα germline transcripts and Iγ-Cμ or Iα-Cμ circle transcripts are given in SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 26. Primer pairs with the sequences Iγ-consensus (SEQ ID NO: 21) and Cγ-consensus (SEQ ID NO: 22) were used for the detection of IgG germline transcript expression; primer pairs with the sequences Iγ-consensus (SEQ ID NO: 21) and Cμ-antisense-1 (SEQ ID NO: 23) were used for the detection of IgG switch circle transcript expression; primer pairs with the sequences Iα-consensus (SEQ ID NO: 24) and Cα-consensus (SEQ ID NO: 25) were used for the detection of IgA germline transcript expression and primer pairs with the sequences Iα-consensus (SEQ ID NO: 24) and Cμ-antisense-2 (SEQ ID NO: 26) were used for the detection of IgA switch circle transcript expression. Data was analysed using the LightCycler Data Analysis software (Roche Applied Science). The results are given as the ratio of the calculated amount of candidate RNA in a given sample by the calculated amount of the housekeeping control β-actin gene in the same sample. β-actin also served as endogenous control and for inter-sample normalisation.

Expression of AID was up-regulated at day 3 of culture in all analysed patient samples (see FIGS. 6A, B and C). Early steps in immunoglobulin isotype switching are characterised by the production of Iγ-Cγ and Iα-Cα germline transcripts, which were detectable in all analysed patient samples (see FIGS. 6A, B and C). When Iγ-Cγ germline transcription was compared with Iα-Cα germline transcription, there was a trend for a reduced expression ratio in all CVID patients. This reflects superior effects of IL-21 plus IL-4 and mAb anti-CD40 stimulation on induction of IgG switching when compared to IgA switching. Switch circle transcripts reflect CSR events and provide a reliable parameter for detection of ongoing CSR. Iγ-Cμ switch circle transcripts were detectable in all CVID patients and Iα-Cμ switch circle transcripts were present in all CVID (see FIGS. 6A and B) and IgAD (see FIG. 6C) patients. Overall, these findings indicate that CSR is induced by a combination of IL-21, IL-4 and anti-CD40 stimulation in CVID and IgAD by up regulation of AID expression and subsequent production of germline transcripts, while the expression of switch circle transcripts did not yet reach its peak level at day 3.

### Example 5

### IL-21 Promotes the Accumulation of Surface IgG+ and IgA+ B Cells in Individuals with CVID or IgAD

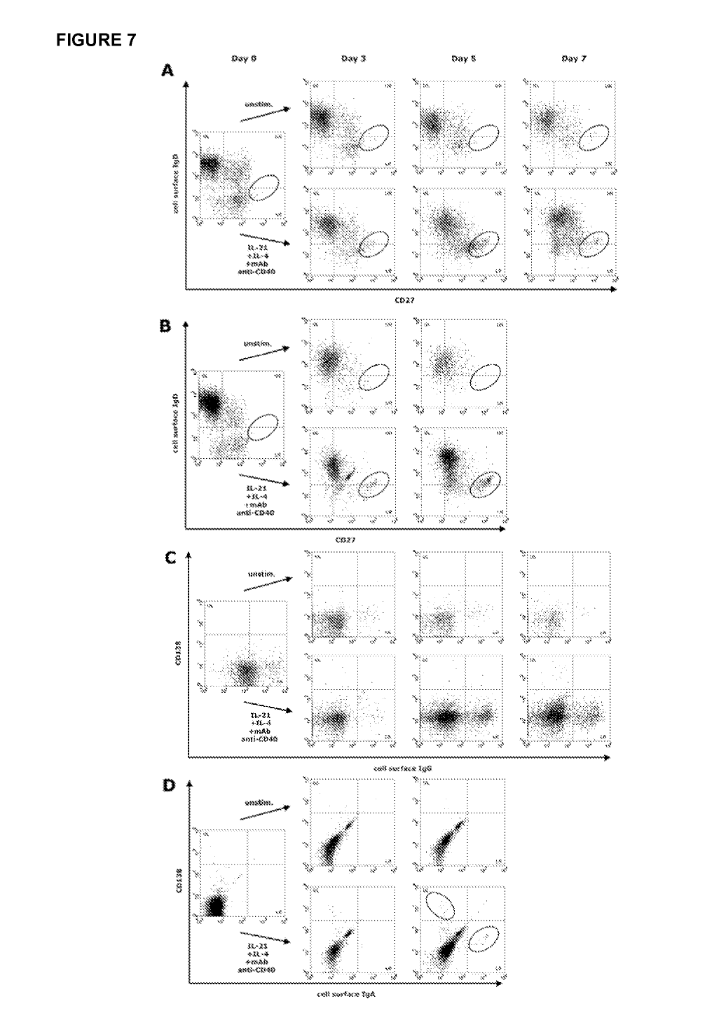

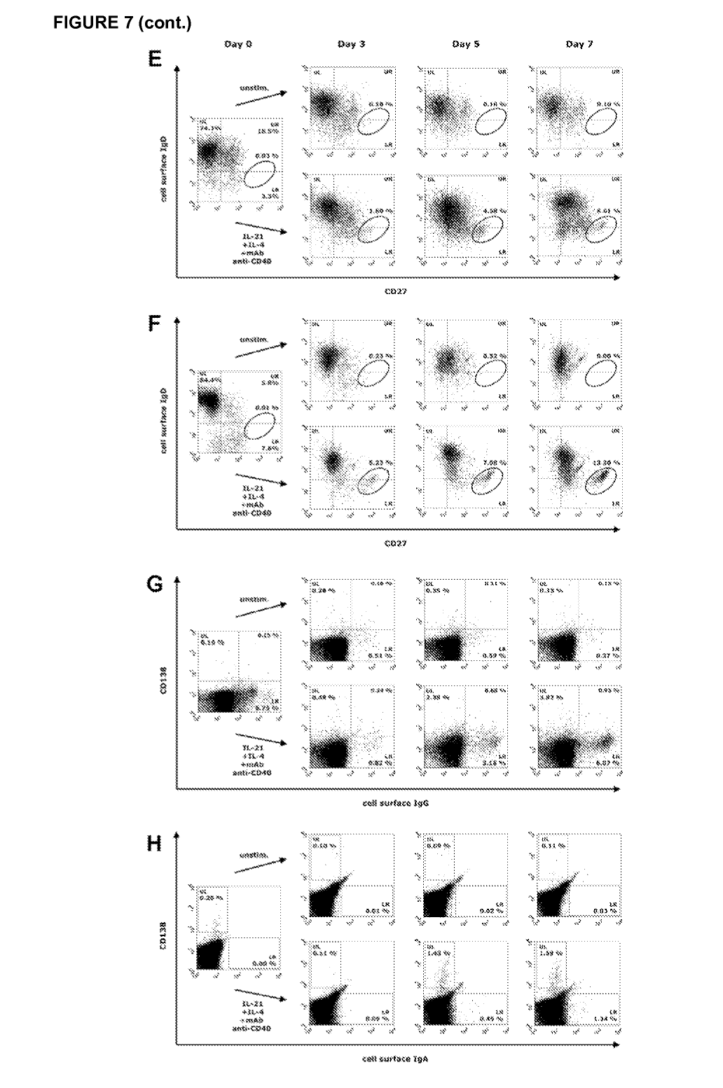

It could be shown that in CVID and IgAD patients a CD27^{high} IgD⁻ CD38^{low} population arises that is recruited from both naïve and memory B cells. Furthermore, a substantial population of sIgG^{high} and sIgA^{high} B cells emerges, accompanied by a population of CD138^{high} plasma cells, albeit smaller than seen in healthy donors (see, for example, FIGS. 7A to H).

Cell Separation and Culture Conditions

To this end heparinised peripheral venous blood was obtained from patients with an established diagnosis of CVID or IgAD, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation and stored in liquid nitrogen until use, according to a method described by Kreher C R et al. (2003) J Immunol Methods 278 (1-2) 79-93. Subsequently, 1×10⁶/ml PBMC were cultured for 7 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, Interleukin-21 was supplemented in a final concentrations of 10 ng/ml and Interleukin-4 in a final concentration of 0.5 ng/ml (both from ImmunoTools, Friesoythe, Germany), as established previously optimal for stimulation of PBMC. Anti-human CD40 monoclonal antibody (clone S2C6) was added at 2 μg/ml (Mabtech AB, Stockholm, Sweden). At days 0, 3, 5, and 7 of culture PBMC were subjected to flow cytometry analysis.

Flow Cytometry Analysis (FCM) and Determination of Cell Subsets

In order to determine the cell subsets a Flow cytometry analysis (FCM) was performed. In particular, single-cell suspensions of cultured PBMC were distributed equally into three 12×75 mm round bottom tubes and washed with 5 ml of phosphate buffered saline (PBS) at 250×g for 5 minutes at room temperature. Subsequently, the following three cocktails of monoclonal antibodies were added each to a single tube to determine B cell and plasma cell populations: (1), anti-CD19-PC7, anti-CD27-FITC (both from DAKO, Glostrup, Denmark), anti-CD5-APC (BD Biosciences) and anti-IgD-PE (SouthernBiotech, Birmingham, Ala., USA). (2), anti-CD19-PC7, anti-IgD-PE, anti-CD38-APC and anti-CD77-FITC (both from BD Biosciences). (3), anti-CD19-PC7, anti-CD138-PE (Miltenyi Biotec, Gladbach, Germany), anti-IgA-FITC and anti-IgG-APC (both from Jackson ImmunoResearch, West Grove, Pa., USA) or anti-CD38-APC. Subsequently, single-cell suspensions were incubated with the cocktails of FACS antibodies for 30 minutes in the dark on ice. Following another washing step with 3 ml of PBS, the remaining cell pellet was resolved in 250 μl of PBS containing 1% formaldehyde and immunophenotyping of lymphocytes was performed by four-colour cytometry on a FACSCalibur (BD Biosciences, Franklin Lakes, N.J., USA) using the CellQuest software (BD Biosciences).

FIG. 7 shows the expression of CD27, CD138 and surface IgD, IgG and IgA on CD19⁺ lymphogated cells in representative individuals with CVID (see FIGS. 7A, C, E and G) or IgAD (see FIGS. 7B, D, F and H). Cell surface expression of these markers is represented on a four-decade log scale as dot plots of correlated x-axis and y-axis fluorescence. In FIGS. 7A, B, E and F quadrant markers were positioned to include naïve mature B cells (UL), natural effector B cells (UR), and IgD⁻ memory B cells (LR). The circle tags a population of CD27^{high} IgD⁻ B cells. In FIGS. 7C and G quadrant markers were positioned to separate CD138^{high} plasma cells (UL) from sIgG^{high} B cells (LR). In FIGS. 7D and H quadrant markers were positioned to separate CD138^{high} plasma cells (UL) from sIgA⁺ B cells (LR).

### Example 6

### Direct Visualization of Ig Producing B Cell Populations in Patients with CVID or IgAD

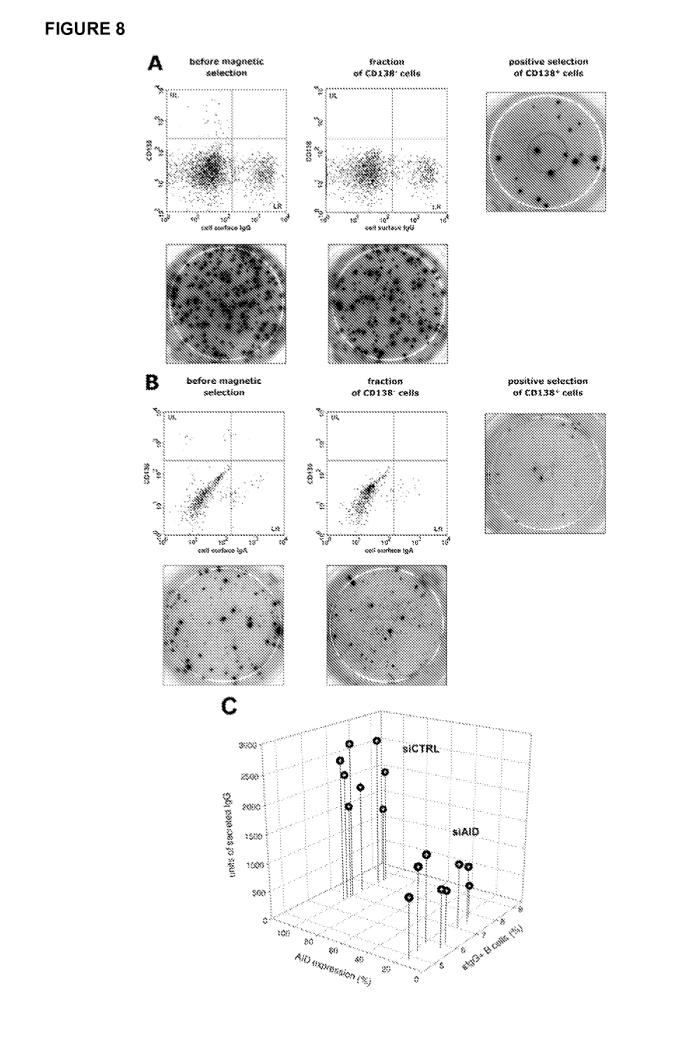

In order to relate IL-21 induced immunoglobulin production to one of these two potential Ig-secreting populations, PBMC were separated by magnetic beads and purified into CD138⁺ plasma cells and CD138⁻ cells, which were subjected to ELISPOT analysis of IgG and IgA secretion (see FIGS. 8A and B). Furthermore, it could be shown that silencing of AID expression only partly abrogates IL-21 driven immunoglobulin production in CVID patients, suggesting a unique mechanism in these patients that is triggered by IL-21 (see FIG. 8C)

Cell Separation and Culture Conditions

To this end heparinised peripheral venous blood was obtained from patients with an established diagnosis of CVID or IgAD, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation and stored in liquid nitrogen until use, according to a method described by Kreher C R et al. (2003) J Immunol Methods 278 (1-2) 79-93. Subsequently, 1×10⁶/ml PBMC were cultured for a total of 5 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, Interleukin-10 and Interleukin-21 were supplemented in a final concentrations of 10 ng/ml and Interleukin-4 in a final concentration of 0.5 ng/ml (both from ImmunoTools, Friesoythe, Germany), as established previously optimal for stimulation of PBMC. If used, anti-human CD40 monoclonal antibody (clone S2C6) was added at 2 μg/ml (Mabtech AB, Stockholm, Sweden). On day 5 of culture, PBMC were washed with twice their culture volume as described above.

Purification of CD138+ Plasma Cells

Plasma cells expressing the CD138 (Syndecan-1) antigen were magnetically purified using the EasySep Human CD138 Positive Selection Kit (StemCell Technologies, Seattle, Wash., USA) according to the manufacturer's protocol. FCM analysis of the positively selected CD138⁺ cells and the CD138⁻ fraction was carried out to verify purification efficacy.

Flow Cytometry Analysis (FCM) and Determination of Cell Subsets

In order to determine the cell subsets a Flow cytometry analysis (FCM) was performed. In particular, single-cell suspensions of PBMC, cultured for 3 days, were distributed into a 12×75 mm round bottom tube and washed with 5 ml of phosphate buffered saline (PBS) at 250×g for 5 minutes at room temperature. Subsequently, a cocktails of monoclonal antibodies was added to the single tube to determine B cell and plasma cell populations: anti-CD19-PC7 (DAKO, Glostrup, Denmark), anti-CD138-PE (Miltenyi Biotec, Gladbach, Germany), anti-IgA-FITC and anti-IgG-APC (both from Jackson ImmunoResearch, West Grove, Pa., USA). Subsequently, single-cell suspensions were incubated with the cocktail of FACS antibodies for 30 minutes in the dark on ice. Following another washing step with 3 ml of PBS, the remaining cell pellet was resolved in 250 μl of PBS containing 1% formaldehyde and immunophenotyping of lymphocytes was performed by four-colour cytometry on a FACSCalibur (BD Biosciences, Franklin Lakes, N.J., USA) using the CellQuest software (BD Biosciences).

Silencing of AID mRNA Expression by RNA Interference

For silencing of activation-induced cytidine deaminase (AID) mRNA expression, a siRNA reagent from Santa Cruz Biotechnology (Santa Cruz, Calif., USA) was used according to the manufacturer's instructions. Briefly, AID siRNA (h, sc-42729) was transfected into 3×10⁵ PBMC, stimulated previously with IL-21, IL-4 and mAb anti-CD40 for 24 hrs. A scrambled siRNA (siRNA-A, sc-37007), not leading to degradation of any known cellular mRNA, was included as a control. RNA interference-mediated knockdown of AID mRNA expression was verified by RT-PCR 72 hrs following transfection. AID expression in control samples was considered as 100% expression level, while samples containing no RNA were treated as blank values (0% expression level).

RNA Isolation and Real-Time Quantitative RT-PCR

Following 72 hrs of culture, RNA was extracted from AID-silenced PBMC using RNeasy Plus Mini Kits (Qiagen, Hilden, Germany). One-step cDNA reverse-transcription and real-time PCR was conducted using the SYBR Green I RNA Master Mix (Roche Applied Science, Mannheim, Germany) and run on a LightCycler 2.0 System (Roche Applied Science). Sequence specific primers for the detection of β-actin and AID expression are given in SEQ ID NO: 17, SEQ ID NO: 18, SEQ ID NO: 19, and SEQ ID NO: 20. Data was analysed using the LightCycler Data Analysis software (Roche Applied Science). The results are given as the ratio of the calculated amount of candidate RNA in a given sample by the calculated amount of the housekeeping control β-actin gene in the same sample. β-actin also served as endogenous control and for inter-sample normalisation.

ELISPOT Assay and Determination of Immunoglobulin Amount

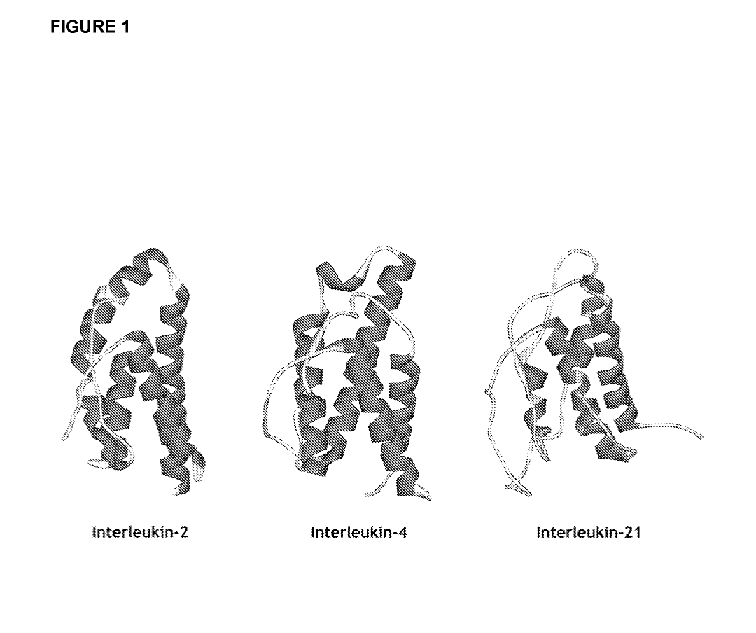

In order to determine the amount of immunoglobulines, an ELISPOT assay was performed. In particular, MultiScreenHTS Filter Plates (Millipore Corp., Bedford, Mass., USA) were pre-wet with 30% ethanol, rinsed three times with sterile PBS (sPBS) and coated overnight at 4° C. with either polyclonal rabbit anti-human IgG or IgA capture antibody (DAKO) diluted in sPBS at 10 μg/ml. After washing, plates were blocked for 3 hrs with sPBS containing 1% bovine serum albumin (SIGMA, St. Louis, Mo., USA). CD138 positive and negative sorted cells (see FIGS. 8A and B) were plated in equal volume-concentrations, according to 5×10⁴ cells for IgG detection (see FIG. 8A) or 1×10⁶ cells for IgA detection (FIG. 8B) in cell culture medium as described above and incubated at 37° C. for 20 hrs in the presence of 5% CO₂. AID-silenced cells (see FIG. 8C) were plated at 4×10⁴ PBMC in cell culture medium as described above and incubated at 37° C. for 20 hrs in the presence of 5% CO₂. Thereafter, the plates were washed six times using PBS containing 0.01% Tween20 (PBS-Tween, SIGMA). Detection antibodies goat anti-human IgG-ALP (Mabtech AB) and goat anti-human IgA-ALP (SouthernBiotech) were diluted in PBS containing 0.5% bovine serum albumin and added at a final concentration of 2 μg/ml. After overnight incubation at 4° C., the plates were washed six times with PBS-Tween. Spot development was carried out using the BCIP/NBT Liquid Substrate System (SIGMA). ELISPOT plate analysis and subsequent enumeration of cell counts and immunoglobulin amount was performed on the AID EliSpot 04 HR Reader using appropriate AID reader software, release 4.0 (Autoimmun Diagnostika GmbH, Strassberg, Germany). The immunoglobulin amount in FIGS. 8A, B and C is measured in a virtual unit that is equivalent to the surface in (0.01 mm)² multiplied by the intensity of a particular spot.

In both CVID and IgAD patients, CD138⁺ plasma cells accounted for only a small fraction of overall IgG or IgA production (see FIGS. 8A and B). FCM data shows that the population of sIgG^{high} and sIgA^{high} B cells is present already at day 3 of culture and therefore these B cells represent already isotype-committed cells that show surface Ig expression and Ig production in response to stimulation with IL-21, IL-4 and anti-CD40 (see FIGS. 8A and B). This unique mechanism of proliferation induced by IL-21, IL-4 and anti-CD40 stimulation in CVID led us to investigate whether silencing of AID expression would prevent production of isotype switched immunoglobulins induced by a combination of IL-21, IL-4 and anti-CD40 stimulation. Strikingly, AID mRNA in patient's PBMC was reduced by >84% at day 3 of culture when compared to siRNA control samples (see FIG. 8C), but numbers of sIgG⁺ B cells within the CD19⁺ lymphogate were only marginally reduced. Furthermore, production of IgG at day 5 of culture was reduced by only ˜58% in AID-silenced PBMC (see FIG. 8C, similar data for IgA production are not shown), suggesting that already switched isotype-committed B cells in CVID are driven to IgG or IgA surface expression and immunoglobulin production by a trigger function of IL-21, IL-4 and anti-CD40 stimulation, that is unique for patients with CVID.

### Example 7

### Expression of IL-21 and IL-21R in Individuals with CVID

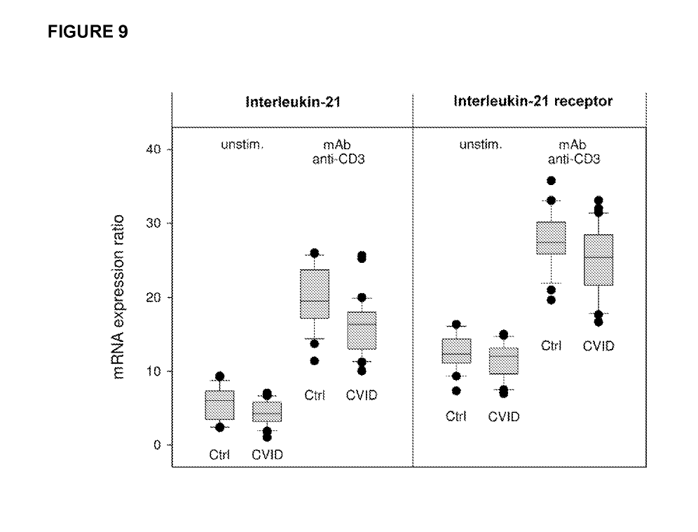

To elucidate whether functional dysregulation of the IL-21/IL-21R system is present in patients with CVID the expression of IL-21 and IL-21 R mRNA upon activation of T cells using a stimulatory anti-human CD3 mAb was studied. Thirty patients with CVID were compared to twenty-two healthy individuals, randomly selected from a pool of blood donors (see FIG. 9).

Expression of IL-21 and IL-21R in Individuals with CVID Cell Separation and Culture Conditions

To this end heparinised peripheral venous blood was obtained from randomly selected healthy donors and patients with an established diagnosis of CVID, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation and stored in liquid nitrogen until use, according to a method described by Kreher C R, et al. (2003) J Immunol Methods 278 (1-2) 79-93. Subsequently, 1×10⁶/ml PBMC were cultured for 14 hrs in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, anti-human CD3 monoclonal antibody (clone CD3-2) was added at 2 μg/ml (Mabtech AB, Stockholm, Sweden).

RNA Isolation and Real-Time Quantitative RT-PCR

Following 14 hrs of culture, RNA was extracted from all PBMC using RNeasy Plus Mini Kits (Qiagen, Hilden, Germany). One-step cDNA reverse-transcription and real-time PCR was conducted using the SYBR Green I RNA Master Mix (Roche Applied Science, Mannheim, Germany) and run on a LightCycler 2.0 System (Roche Applied Science). Sequence specific primers for the detection of β-actin, IL-21, and IL-21R expression are given in SEQ ID NO: 17, SEQ ID NO: 18, SEQ ID NO: 27, SEQ ID NO: 28, SEQ ID NO: 29, and SEQ ID NO: 30, respectively. Data was analysed using the LightCycler Data Analysis software (Roche Applied Science). The results are given as the ratio of the calculated amount of candidate RNA in a given sample by the calculated amount of the housekeeping control β-actin gene in the same sample. β-actin also served as endogenous control and for inter-sample normalisation.

Analysis of IL-21 and IL-21 R mRNA expression upon anti-CD3 stimulation of T cells showed no evidence for defective IL-21 or IL-21 R expression in patients with CVID (see FIG. 9), thus providing the functional basis for a therapeutic role of IL-21 in CVID.

### Example 8

### IL-21 Induces Tetanus and Diphtheria Toxoid-Specific IgG in Patients with CVID

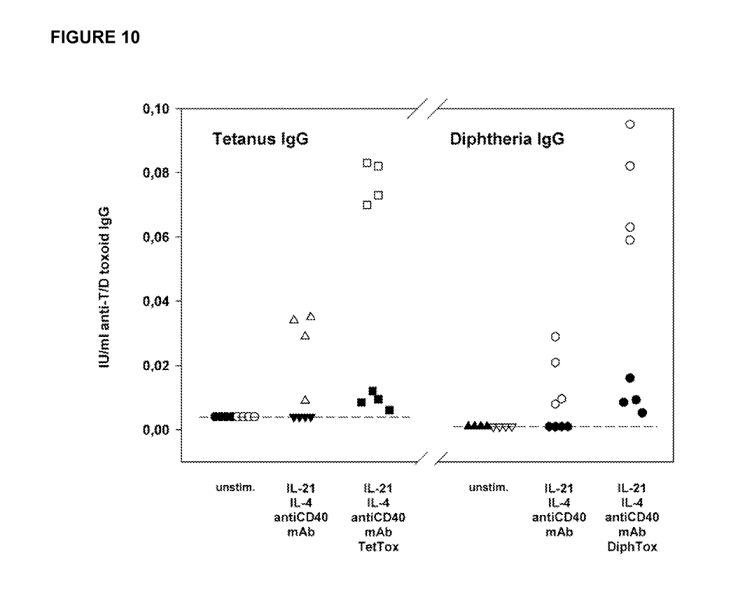

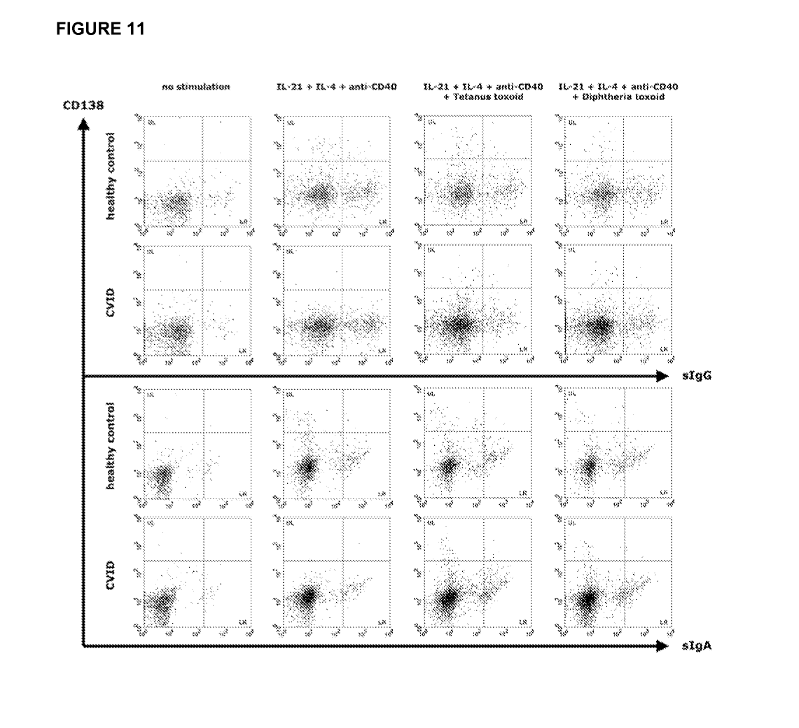

To investigate the ability of IL-21 to induce vaccine-like antigen-specific immunoglobulin production in vitro, PBMC from CVID patients were stimulated for 7 days in the presence of IL-21, IL-4, anti-CD40 mAb and either diphtheria or tetanus toxoid, being virtually the essential part of the respective vaccines (see FIGS. 10 and 11).

Cell Separation and Culture Conditions

To this end heparinised peripheral venous blood was obtained from patients with an established diagnosis of CVID, according to the criteria of the European Society for Immunodeficiency Diseases (ESID), “Diagnostic criteria for PID”. http://www.esid.org/workingparty.php?party=3&sub=2&id=73; accessed on Oct. 12, 2008. PBMC were isolated by Ficoll separation and stored in liquid nitrogen until use, according to a method described by Kreher C R et al. (2003) J Immunol Methods 278 (1-2) 79-93. Subsequently, 2×10⁶/ml PBMC were cultured for 7 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, Interleukin-10 and Interleukin-21 were supplemented in a final concentrations of 10 ng/ml and Interleukin-4 in a final concentration of 0.5 ng/ml (both from ImmunoTools, Friesoythe, Germany), as established previously optimal for stimulation of PBMC. Anti-human CD40 monoclonal antibody (clone S2C6) was added at 2 μg/ml (Mabtech AB, Stockholm, Sweden). Tetanus and diphtheria toxoid was purchased from Statens Serum Institut (Copenhagen, Denmark) and added in concentrations previously established optimal for PBMC. On day 7 of culture, PBMC and all of their culture medium were centrifuged at 250×g for 10 minutes at room temperature and the medium supernatant was removed for analysis. Subsequently, the cell pellet of PBMC was washed with twice its culture volume as described above.

ELISA Protocol and Determination of Tetanus and Diphtheria Toxoid-Specific IgG

In order to determine Tetanus and Diphtheria toxoid-specific IgGs, IgG antibodies to tetanus toxoid and diphtheria toxoid were quantified in medium supernatants at day 7 of 2×10⁶ cultured PBMC using VaccZyme Tetanus toxoid IgG or VaccZyme Diphtheria toxoid IgG assays (The Binding Site, Birmingham, England). ELISA protocols and calibrators were modified to allow detection of very low amounts of toxoid specific IgG antibodies (>0.004 IU/ml anti-tetanus toxoid IgG, >0.001 IU/ml anti-diphtheria toxoid IgG). The absorbance of all ELISA plate samples was measured at 450 nm and analysed with a SPECTRA Classic micro plate reader and appropriate reader software (TECAN Trading, Salzburg, Austria).

Flow Cytometry Analysis (FCM) and Determination of Cell Subsets

In order to determine the cell subsets a Flow cytometry analysis (FCM) was performed. In particular, single-cell suspensions of PBMC, cultured for 7 days, were distributed into 12×75 mm round bottom tubes and washed with 5 ml of phosphate buffered saline (PBS) at 250×g for 5 minutes at room temperature. Subsequently, a cocktail of monoclonal antibodies was added to the single tubes to determine B cell and plasma cell populations: anti-CD19-PC7 (DAKO, Glostrup, Denmark), anti-CD138-PE (Miltenyi Biotec, Gladbach, Germany), anti-IgA-FITC and anti-IgG-APC (both from Jackson ImmunoResearch, West Grove, Pa., USA). Subsequently, single-cell suspensions were incubated with the cocktail of FACS antibodies for 30 minutes in the dark on ice. Following another washing step with 3 ml of PBS, the remaining cell pellet was resolved in 250 μl of PBS containing 1% formaldehyde and immunophenotyping of lymphocytes was performed by four-colour cytometry on a FACSCalibur (BD Biosciences, Franklin Lakes, N.J., USA) using the CellQuest software (BD Biosciences).

In CVID patients, a small amount of specific-antibodies was detected in samples stimulated with IL-21, IL-4, anti-CD40 mAb, and diphtheria or tetanus toxoid for 7 days (see FIG. 10). FCM analysis of antigen-challenged PBMC from CVID patients revealed IL-21-induced generation of CD138⁺ plasma cells and enhanced formation of sIgG^{high} and sIgA^{high} B cells (see FIG. 11). In contrast, antigen-free IL-21 stimulation did not lead to CD138⁺ plasma cell accumulation in cultures of cells from CVID patients.

### Example 9

### IL-21 Variants Induce IgG and IgA Production in Purified Human B Cells from Patients with CVID

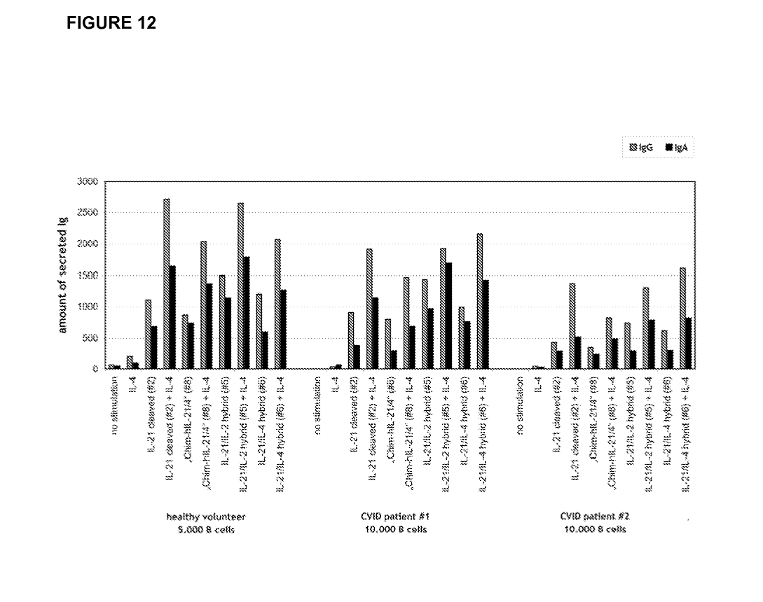

In the following experiment the efficacy of various IL-21 variants with regard to their potency to induce IgG and IgA production in anti-CD40 stimulated purified CD19⁺ B cells from patients with CVID was compared (see, for example, FIG. 12).

Preparation of Plasmid DNA from IL-21 Variants

To this end, synthetic genes of IL-21 variants were assembled from synthetic oligonucleotides and PCR products according to the DNA sequences given in SEQ ID NO: 31, SEQ ID NO: 32, SEQ ID NO: 33, and SEQ ID NO: 34; wherein SEQ ID NO:31 represents the cleaved version of IL-21 with the modification of a N-terminal hexa histidine-tag followed by a Factor Xa Protease recognition site. SEQ ID NO:32 refers to a protein described in a publication by Kent Bondensgaard (Bondensgaard, K., et al. (2007) J Biol Chem. 282, 23326-36) and termed “Chim-hIL-21/4”, that was also modified at the N-terminus with a hexa histidine-tag followed by a Factor Xa Protease recognition site; the original protein sequence of “Chim-hIL-21/4” has been indicated as SEQ ID NO: 9 in the sequence listing, and the corresponding DNA sequence has been indicated as SEQ ID NO: 16 in the sequence listing. Similarly, both IL-21 variants SEQ ID NO: 33 and SEQ ID NO: 34 were modified at the N-terminus with a hexa histidine-tag followed by a Factor Xa Protease recognition site. Synthetic genes of IL-21 variants were sent out for cloning into pET45b(+) (ampR) vectors (Novagen, Gibbstown, N.J., USA) using XbaI and XhoI restriction sites (imaGenes, Berlin, Germany). The final construct was verified by sequencing and the sequence congruence within the used restriction sites and the originally desired DNA sequences was 100% (imaGenes, Berlin, Germany). The plasmid DNA was purified from transformed bacteria using the Pure Yield™ Plasmid Midiprep System (Promega, Madison, Wis., USA) according to the manufacturer's instructions, and concentration was determined by UV spectroscopy.

In Vitro Synthesis and Purification of IL-21 Variant Proteins

E. coli-based in vitro synthesis of IL-21 variant proteins was done using the EasyXpress Protein Synthesis Mini Kit (Qiagen, Hilden, Germany) according to the manufacturer's instructions. Subsequently, the final reaction volume was subjected to Ni-NTA based purification for 6× His-tagged proteins under native conditions using Ni-NTA spin columns (Qiagen, Hilden, Germany). The hexa histidine-tag was cleaved from purified IL-21 variants using a Factor Xa Protease (Qiagen, Hilden, Germany) and removed from the reaction volume by either standard SDS page electrophoresis or another round of Ni-NTA spin column passage. The concentration of the cleaved IL-21 variant proteins was determined by standard Bradford protein assay (Bradford, M. (1976). Anal. Biochem. 72, 248-254) and adjusted to a concentration of 1 mg/ml protein in phosphate buffered saline (PBS) containing 0.5% bovine serum albumine (BSA).

Cell Separation, Purification of CD19+ B Cells and Culture Conditions

In order to separate and purify CD19⁺ B cells heparinised peripheral venous blood was obtained from healthy volunteers and patients with an established diagnosis of CVID, according to the criteria of the European Society for Immunodeficiency Diseases (ESID), “Diagnostic criteria for PID”. http://www.esid.org/workingparty.php?party=3&sub=2&id=73; accessed on Oct. 12, 2008. PBMC were isolated by Ficoll separation, according to a method described by Kreher C R et al. (2003) J Immunol Methods 278 (1-2) 79-93. Subsequently, B cells expressing the CD19 antigen were magnetically purified using the EasySep Human CD19 Positive Selection Kit (StemCell Technologies, Seattle, Wash., USA) according to the manufacturer's protocol. FCM analysis of the positively selected CD19⁺ cells and the CD19⁻ fraction was carried out to verify purification efficacy. Subsequently, 1×10⁵ B cells/ml were stimulated for 5 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, IL-21 variant proteins were supplemented in a final concentration of 10 ng/ml and anti-CD40 mAb (clone S2C6, Mabtech AB, Stockholm, Sweden) was added in a final concentration of 2 μg/ml. If used, Interleukin-4 was added in a final concentration of 0.5 ng/ml. On day 5 of culture, B cells were washed with twice their culture volume as described above.

ELISPOT Assay and Determination of Immunoglobulin Amount

In order to determine the amount of immunoglobulines, an ELISPOT assay was performed. In particular, MultiScreenHTS Filter Plates (Millipore Corp., Bedford, Mass., USA) were pre-wet with 30% ethanol, rinsed three times with sterile PBS (sPBS) and coated overnight at 4° C. with either polyclonal rabbit anti-human IgG or IgA capture antibody (DAKO) diluted in sPBS at 10 μg/ml. After washing, plates were blocked for 3 hrs with sPBS containing 1% bovine serum albumin (SIGMA, St. Louis, Mo., USA). Cultured B cells were plated at 1×10⁴ cells/well in cell culture medium as described above and incubated at 37° C. for 20 hrs in the presence of 5% CO₂. Thereafter, the plates were washed six times using PBS containing 0.01% Tween20 (PBS-Tween, SIGMA). Detection antibodies goat anti-human IgG-ALP (Mabtech AB) and goat anti-human IgA-ALP (SouthernBiotech) were diluted in PBS containing 0.5% bovine serum albumin and added at a final concentrations of 2 μg/ml. After overnight incubation at 4° C., the plates were washed six times with PBS-Tween. Spot development was carried out using the BCIP/NBT Liquid Substrate System (SIGMA). ELISPOT plate analysis and subsequent enumeration of cell counts and immunoglobulin amount was performed on the AID EliSpot 04 HR Reader using appropriate AID reader software, release 4.0 (Autoimmun Diagnostika GmbH, Strassberg, Germany). The immunoglobulin amount in FIGS. 3A and B is measured in a virtual unit that is equivalent to the surface in (0.01 mm)² multiplied by the intensity of a particular spot.

When employed in healthy volunteers, IL-21 variant proteins with the sequences SEQ ID NO: 33 and SEQ ID NO: 34 induced similar to higher amounts of IgG and IgA production in comparison to mature, cleaved IL-21 (SEQ ID NO: 31) when B cells were cultured without additional IL-4 (see FIG. 12). An IL-21 variant with the sequence SEQ ID NO: 32, that was described in a publication by Kent Bondensgaard (Bondensgaard, K., et al. (2007) J Biol Chem. 282, 23326-36), generally induced lower amounts of secreted IgG or IgA. When IL-4 was added, Ig production as generally increased. In patients with CVID, IL-21 variant proteins with the sequences SEQ ID NO: 33 and SEQ ID NO: 34 showed even more potency to induce IgG and IgA production, when compared to mature, cleaved IL-21 or the IL-21 variant with the sequence SEQ ID NO: 32 and when B cells were cultured without additional IL-4 (see FIG. 12). Similar to healthy volunteers, addition of IL-4 increased the amount of Ig production overall.

### Example 10

### IL-21 and IL-21 Variants Cooperate with Galectin-1 and Galectin-3 in Synergy to Induce the Production of IgG and IgA in Patients with CVID

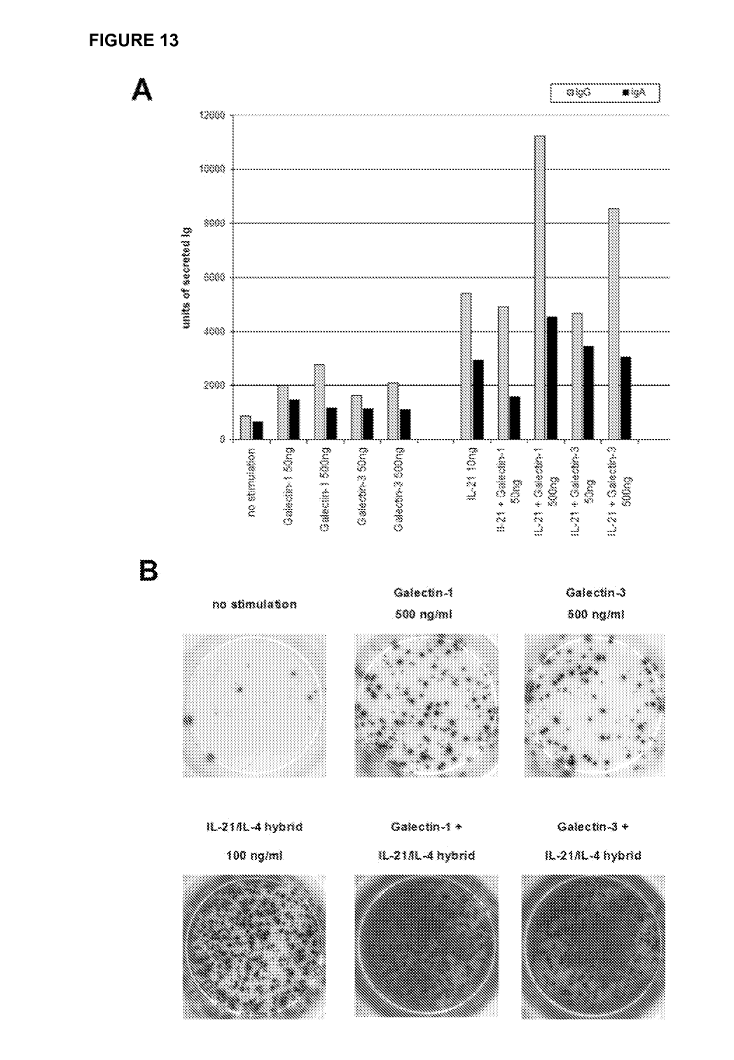

In the following experiment the efficacy of IL-21 and the IL-21/IL-4 hybrid variant with regard to their potency to induce IgG and IgA production in combination with Galectin-1 and Galectin-3 was shown in anti-CD40 stimulated peripheral blood mononuclear cells (PBMC) purified B cells or from patients with CVID (see, for example, FIGS. 13A and B).

Preparation of Plasmid DNA from IL-21 Variants

To this end, synthetic genes of IL-21 variants were assembled from synthetic oligonucleotides and PCR products according to the DNA sequences given in SEQ SEQ ID NO: 33, and SEQ ID NO: 34; wherein both IL-21 variants were modified at the N-terminus with a hexa histidine-tag followed by a Factor Xa Protease recognition site. Synthetic genes of IL-21 variants were sent out for cloning into pET45b(+) (ampR) vectors (Novagen, Gibbstown, N.J., USA) using XbaI and XhoI restriction sites (imaGenes, Berlin, Germany). The final construct was verified by sequencing and the sequence congruence within the used restriction sites and the originally desired DNA sequences was 100% (imaGenes, Berlin, Germany). The plasmid DNA was purified from transformed bacteria using the Pure Yield™ Plasmid Midiprep System (Promega, Madison, Wis., USA) according to the manufacturer's instructions, and concentration was determined by UV spectroscopy.

In Vitro Synthesis and Purification of IL-21 Variant Proteins

E. coli-based in vitro synthesis of IL-21 variant proteins was done using the EasyXpress Protein Synthesis Mini Kit (Qiagen, Hilden, Germany) according to the manufacturer's instructions. Subsequently, the final reaction volume was subjected to Ni-NTA based purification for 6× His-tagged proteins under native conditions using Ni-NTA spin columns (Qiagen, Hilden, Germany). The hexa histidine-tag was cleaved from purified IL-21 variants using a Factor Xa Protease (Qiagen, Hilden, Germany) and removed from the reaction volume by either standard SDS page electrophoresis or another round of Ni-NTA spin column passage. The concentration of the cleaved IL-21 variant proteins was determined by standard Bradford protein assay (Bradford, M. (1976). Anal. Biochem. 72, 248-254) and adjusted to a concentration of 1 mg/ml protein in phosphate buffered saline (PBS) containing 0.5% bovine serum albumine (BSA).

Cell Separation, Purification of CD19+ B Cells and Culture Conditions

To this end heparinised peripheral venous blood was obtained from patients with an established diagnosis of CVID or IgAD, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation and stored in liquid nitrogen until use, according to a method described by Kreher C R et al. (2003) J Immunol Methods 278 (1-2) 79-93. In order to separate and purify CD19+ B cells, the EasySep Human CD19 Positive Selection Kit (StemCell Technologies, Seattle, Wash., USA) was used according to the manufacturer's protocol. FCM analysis of the positively selected CD19+ cells and the CD19-fraction was carried out to verify purification efficacy. Subsequently, 5×10⁵/ml PBMC or 2×10⁵ B cells/ml were cultured for 5 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, IL-21 (ImmunoTools, Friesoythe, Germany) was supplemented in a final concentration of either 10 ng/ml or 100 ng/ml and IL-21 variant proteins were supplemented in a final concentration of 100 ng/ml and anti-CD40 mAb (clone S2C6, Mabtech AB, Stockholm, Sweden) was added in a final concentration of 2 μg/ml. In addition, Galectin-1 and Galectin-3 were supplemented in a final concentration of 50 ng/ml or 500 ng/ml (both from Biomol, Hamburg, Germany). On day 5 or day 7 of culture, PBMC or B cells were washed with twice their culture volume as described above.

ELISPOT Assay and Determination of Immunoglobulin Amount

In order to determine the amount of immunoglobulines, an ELISPOT assay was performed. In particular, MultiScreenHTS Filter Plates (Millipore Corp., Bedford, Mass., USA) were pre-wet with 30% ethanol, rinsed three times with sterile PBS (sPBS) and coated overnight at 4° C. with either polyclonal rabbit anti-human IgG or IgA capture antibody (DAKO) diluted in sPBS at 10 μg/ml. After washing, plates were blocked for 3 hrs with sPBS containing 1% bovine serum albumin (SIGMA, St. Louis, Mo., USA). Cultured PBMC were plated at 2.5×10⁵ cells/well and B cells were plated at 5×10⁴ cells/well in cell culture medium as described above and incubated at 37° C. for 20 hrs in the presence of 5% CO₂. Thereafter, the plates were washed six times using PBS containing 0.01% Tween20 (PBS-Tween, SIGMA). Detection antibodies goat anti-human IgG-ALP (Mabtech AB) and goat anti-human IgA-ALP (SouthernBiotech) were diluted in PBS containing 0.5% bovine serum albumin and added at a final concentrations of 2 μg/ml. After overnight incubation at 4° C., the plates were washed six times with PBS-Tween. Spot development was carried out using the BCIP/NBT Liquid Substrate System (SIGMA). ELISPOT plate analysis and subsequent enumeration of cell counts and immunoglobulin amount was performed on the AID EliSpot 04 HR Reader using appropriate AID reader software, release 4.0 (Autoimmun Diagnostika GmbH, Strassberg, Germany). The immunoglobulin amount in FIG. 13A is measured in a virtual unit that is equivalent to the surface in (0.01 mm)² multiplied by the intensity of a particular spot. Single-well photos were shown FIG. 13B, that depict the results of ELISPOT assays, whilst every dark spot represents a single IgG-producing B cell.

Galectin-1 and Galectin-3 alone only marginally induced IgG or IgA production in PBMC from patients with CVID within a broad range of Galectin-1 or Galectin-3 concentrations, respectively. Addition of IL-21 to Galectin-1 or Galectin-3 stimulated PBMC gave rise to even higher amounts of secreted IgG or IgA when compared to IL-21 alone. When the IL-21/IL-4 hybrid variant was combined with either Galectin-1 or Galectin-3 the production of IgG from purified CD19⁺ B cells from patients with CVID was markedly potentiated. These experiments outline the potential of a combination of IL-21 or IL-21 variants and Galectin-1 or Galectin-3 to restore immunoglobuline production in patients with primary humoral immunodeficiency diseases.

### Example 11

### IGIP and Syntenin-1 Potentiate the Effects of IL-21 Variants to Induce IgA Production in Patients with CVID

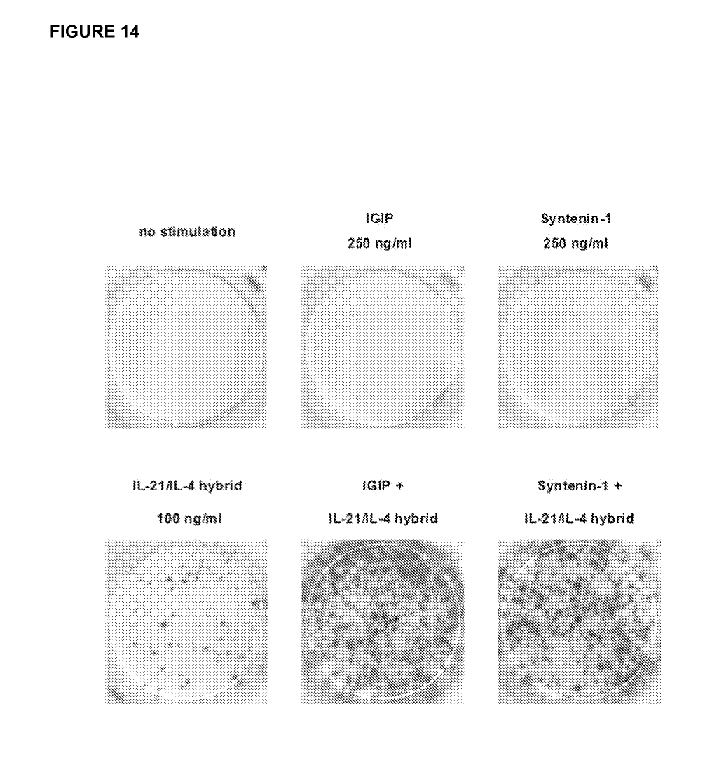

In the following experiment the efficacy of IGIP (IgA-inducing protein,) IL-21/IL-4 hybrid variants was shown with regard to their potency to induce IgG and IgA production in combination with Galectin-1 and Galectin-3 was shown in anti-CD40 stimulated peripheral blood mononuclear cells (PBMC) purified B cells or from patients with CVID (see, for example, FIG. 14).

Preparation of Plasmid DNA from IL-21 Variants

To this end, synthetic genes of IL-21 variants were assembled from synthetic oligonucleotides and PCR products according to the DNA sequences given in SEQ SEQ ID NO: 33, and SEQ ID NO: 34; wherein both IL-21 variants were modified at the N-terminus with a hexa histidine-tag followed by a Factor Xa Protease recognition site. Synthetic genes of IL-21 variants were sent out for cloning into pET45b(+) (ampR) vectors (Novagen, Gibbstown, N.J., USA) using XbaI and XhoI restriction sites (imaGenes, Berlin, Germany). The final construct was verified by sequencing and the sequence congruence within the used restriction sites and the originally desired DNA sequences was 100% (imaGenes, Berlin, Germany). The plasmid DNA was purified from transformed bacteria using the Pure Yield™ Plasmid Midiprep System (Promega, Madison, Wis., USA) according to the manufacturer's instructions, and concentration was determined by UV spectroscopy.

In Vitro Synthesis and Purification of IL-21 Variant Proteins

E. coli-based in vitro synthesis of IL-21 variant proteins was done using the EasyXpress Protein Synthesis Mini Kit (Qiagen, Hilden, Germany) according to the manufacturer's instructions. Subsequently, the final reaction volume was subjected to Ni-NTA based purification for 6× His-tagged proteins under native conditions using Ni-NTA spin columns (Qiagen, Hilden, Germany). The hexa histidine-tag was cleaved from purified IL-21 variants using a Factor Xa Protease (Qiagen, Hilden, Germany) and removed from the reaction volume by either standard SDS page electrophoresis or another round of Ni-NTA spin column passage. The concentration of the cleaved IL-21 variant proteins was determined by standard Bradford protein assay (Bradford, M. (1976), Anal. Biochem. 72, 248-254) and adjusted to a concentration of 1 mg/ml protein in phosphate buffered saline (PBS) containing 0.5% bovine serum albumine (BSA).

Preparation of Plasmid DNA from IGIP and Syntenin-1

To this end, synthetic genes for IGIP and Syntenin-1 were assembled by using a complementary DNA (cDNA) library from enriched human PBMC or lymph node lymphocytes. The synthetic gene for IGIP was amplified from the chromosome 5 open reading frame 53 (C5orf53, NCBI reference sequence NM_{—}001007189). The synthetic gene for Syntenin-1 was amplified from the coding region of human Syntenin-1 (Genbank Acc. number BC013254). Human IGIP and Syntenin-1 cDNA, with the addition of a hexa histidine-tag followed by a Factor Xa Protease recognition site, was inserted into the pTUAT vector. The IGIP hexa histidine-tag or Syntenin-1 hexa histidine-tag containing DNA fragments were then cloned into the Escherichia coli/Lactobacillus shuttle vector pLP402, forming pLP402-IGIP and pLP402-Synth. A terminator from the lactate dehydrogenase gene (Tldh) was present between the C-terminal region of amylase and the N-terminal of the IGIP hexa histidine-tag or Syntenin-1 hexa histidine-tag constructs to suppress the expression of the down-stream sequences in E. coli. The Tldh was removed by NotI digestion of the plasmid, and after ligation the resulting vectors were introduced into Lactobacillus zeae ATCC 393 (L. zeae).

Lactobacillus Expression and Purification of IGIP and Syntenin-1 Proteins

L. zeae, transformed with either the plasmids pLP402-IGIP or pLP402-Synth, were selected on MRS (Difco) plates with 3 μg/ml erythromycin after cultivation anaerobically at 37° C. for 48 h. The pLP402-IGIP and pLP402-Synth vectors, respectively mediated the secretion of IGIP and Syntenin-1 proteins into the medium under the transcriptional control of the regulatable α-amylase promoter. The α-amylase promoter is regulated by a negative feedback. It is repressed by PTS sugars such as glucose and lactose in L. zeae. Growth in presence of non-PTS sugars, such as mannitol, de-represses the promoter and activates gene expression. Cell culture medium was harvested in the exponential growth phase at an optical density at 600 nm (OD600) of 0.8 (10⁸ cfu/ml). Subsequently, the cell culture medium was subjected to Ni-NTA based purification for 6× His-tagged proteins under native conditions using Ni-NTA spin columns (Qiagen, Hilden, Germany). The hexa histidine-tag was cleaved from purified IGIP and Syntenin-1 proteins using a Factor Xa Protease (Qiagen, Hilden, Germany) and removed from the reaction volume by either standard SDS page electrophoresis or another round of Ni-NTA spin column passage. The concentration of cleaved IGIP and Syntenin-1 proteins was determined by standard Bradford protein assay (Bradford, M. (1976) Anal. Biochem. 72, 248-254) and adjusted to a concentration of 1 mg/ml protein in phosphate buffered saline (PBS) containing 0.5% bovine serum albumine (BSA).

Cell Separation, Purification of CD19+ B Cells and Culture Conditions

In order to separate and purify CD19+ B cells heparinised peripheral venous blood was obtained from healthy volunteers and patients with an established diagnosis of CVID, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation, according to a method described by Kreher C R, et al. (2003) J Immunol Methods 278 (1-2) 79-93. Subsequently, B cells expressing the CD19 antigen were magnetically purified using the EasySep Human CD19 Positive Selection Kit (StemCell Technologies, Seattle, Wash., USA) according to the manufacturer's protocol. FCM analysis of the positively selected CD19⁺ cells and the CD19-fraction was carried out to verify purification efficacy. Subsequently, 4×10⁵ B cells/ml were stimulated for 7 days in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. In addition, IL-21/IL-4 hybrid variant protein was supplemented in a final concentration of 100 ng/ml and IGIP protein or Syntenin-1 protein was added in a final concentration of 250 ng/ml. On day 7 of culture, B cells were washed with twice their culture volume as described above.

ELISPOT Assay and Determination of Immunoglobulin amount

In order to determine the amount of IgA, an ELISPOT assay was performed. In particular, MultiScreenHTS Filter Plates (Millipore Corp., Bedford, Mass., USA) were pre-wet with 30% ethanol, rinsed three times with sterile PBS (sPBS) and coated overnight at 4° C. with polyclonal rabbit anti-human IgA capture antibody (DAKO) diluted in sPBS at 10 μg/ml. After washing, plates were blocked for 3 hrs with sPBS containing 1% bovine serum albumin (SIGMA, St. Louis, Mo., USA). Cultured B cells were plated at 2.5×10⁵ cells/well in cell culture medium as described above and incubated at 37° C. for 20 hrs in the presence of 5% CO₂. Thereafter, the plates were washed six times using PBS containing 0.01% Tween20 (PBS-Tween, SIGMA). The detection antibody goat anti-human IgA-ALP (SouthernBiotech) was diluted in PBS containing 0.5% bovine serum albumin and added at a final concentrations of 2 μg/ml. After overnight incubation at 4° C., the plates were washed six times with PBS-Tween. Spot development was carried out using the BCIP/NBT Liquid Substrate System (SIGMA). ELISPOT plate analysis and subsequent enumeration of cell counts and immunoglobulin amount was performed on the AID EliSpot 04 HR Reader using appropriate AID reader software, release 4.0 (Autoimmun Diagnostika GmbH, Strassberg, Germany). Single-well photos were shown FIG. 14, that depict the results of ELISPOT assays, whilst every dark spot represents a single IgA-producing B cell.

IGIP and Syntenin-1 proteins alone only induced very slight IgA production in B cells from CVID patients following an incubation period of 7 days, whilst stimulation with IL-21/IL-4 hybrid variant protein led to a marked increase in IgA production in comparison. When the IL-21/IL-4 hybrid variant was combined with either IGIP or Syntenin-1, the production of IgA from purified CD19⁺ B cells from patients with CVID was unexpectedly increased several-fold. These experiments outline the potential of a combination of IL-21 variants and IGIP or Syntenin-1 to restore immunoglobuline production in patients with primary humoral immunodeficiency diseases.

### Example 12

### Lactobacilli can Act as Live Carriers of Surface-Expressed IL-21 Variants and CD40L Molecules to Induce IgG and IgA Production in Patients with CVID or IgAD

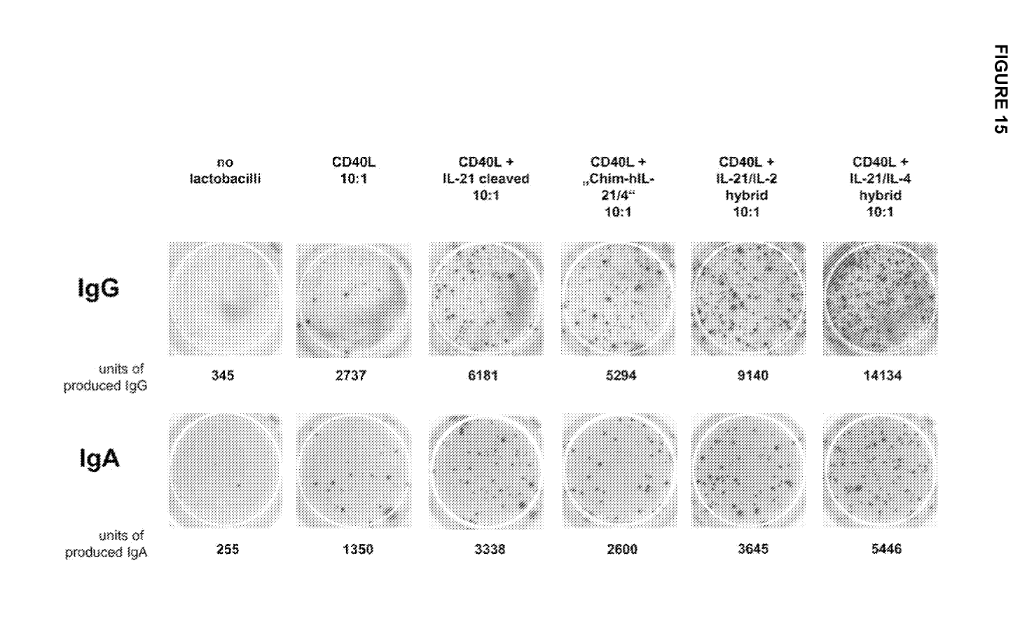

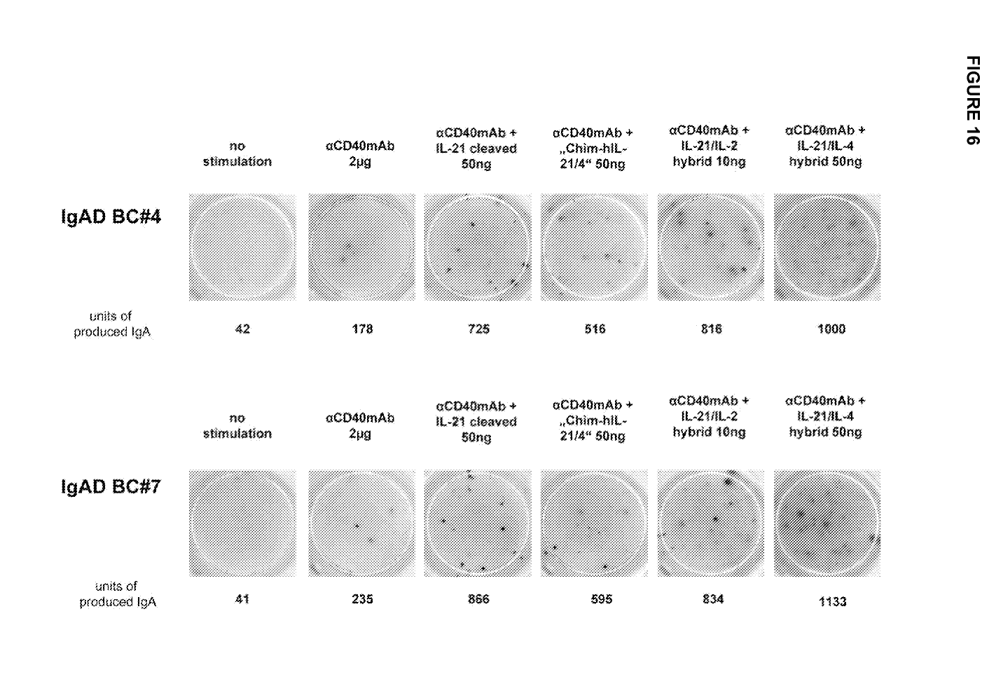

In the following experiment, lactobacilli as live carriers of surface-expressed IL-21 variants and CD40L molecules were assayed for their potency to induce IgG and IgA production of PBMC from patients with CVID or IgAD in a close-to-in-vivo setting (see, for example, FIGS. 15, 16, and 17).

Preparation of Plasmid DNA from IL-21 Variants and CD40L

To this end, synthetic genes of mature cleaved IL-21, “Chim-hIL-21/4” and IL-21 variants were assembled from synthetic oligonucleotides and PCR products according to the DNA sequences given in SEQ ID NO: 31, SEQ ID NO: 9, SEQ SEQ ID NO: 33, and SEQ ID NO: 34; wherein both IL-21 variants were relieved before assembly from amino acids encoding for the hexa histidine-tag followed by the Factor Xa Protease recognition site. A synthetic gene for CD40L was assembled by using a complementary DNA (cDNA) library from enriched human PBMC. The synthetic gene for CD40L was amplified from the coding region of human CD40LG (UniGene Hs. 592244). The synthetic genes of mature cleaved IL-21, “Chim-hIL-21/4”, IL-21/IL-2 hybrid protein, IL-21/IL-4 hybrid protein and CD40L, with addition of an E-tag encoding sequence, were separately inserted into the vector pTUAT. A short or a long anchor sequence of the proteinase P-encoding gene PrtP was introduced, generating p-TUAT-Ank (anchor). The synthetic genes of mature cleaved IL-21-anchor-containing, “Chim-hIL-21/4”-anchor-containing, IL-21/IL-2 hybrid protein-anchor-containing, IL-21/IL-4 hybrid protein-anchor-containing and CD40L-anchor-containing DNA fragments were then separately cloned into the Escherichia coli/Lactobacillus shuttle vector pLP402. A terminator from the lactate dehydrogenase gene (Tldh) was present between the C-terminal region of amylase and the N-terminal of the IGIP hexa histidine-tag or Syntenin-1 hexa histidine-tag constructs to suppress the expression of the down-stream sequences in E. coli. The Tldh was removed by NotI digestion of the plasmid, and after ligation the resulting vectors were introduced into Lactobacillus zeae ATCC 393 (L. zeae). Two different constructs were made: (i) the pLP402 containing mature cleaved IL-21-short-anchor, “Chim-hIL-21/4”-short-anchor, IL-21/IL-2 hybrid protein-short-anchor, IL-21/IL-4 hybrid protein-short-anchor and CD40L-short-anchor versions, mediating cell surface expression by fusion to the last 117 amino acids of the proteinase P of L. zeae; the pLP402 containing mature cleaved IL-21-long-anchor, “Chim-hIL-21/4”-long-anchor, IL-21/IL-2 hybrid protein-long-anchor, IL-21/IL-4 hybrid protein-long-anchor and CD40L-long-anchor versions, mediating cell surface expression by fusion to the last 244 amino acids of the proteinase P protein.

Lactobacillus Expression of Mature Cleaved IL-21, “Chim-hIL-21/4”, IL-21 Variants and CD40L and Enumeration of Lactobacilli for Cell Culture

L. zeae, transformed with the plasmids pLP402, were selected on MRS (Difco) plates with 3 μg/ml erythromycin after cultivation anaerobically at 37° C. for 48 h. The pLP402 vectors mediated the surface-anchored expression of mature cleaved IL-21, “Chim-hIL-21/4”, IL-21 variants and CD40L proteins under the transcriptional control of the regulatable α-amylase promoter. The α-amylase promoter is regulated by a negative feedback. It is repressed by PTS sugars such as glucose and lactose in L. zeae. Growth in presence of non-PTS sugars, such as mannitol, de-represses the promoter and activates gene expression. Cells were harvested in the exponential growth phase at an optical density at 600 nm (OD600) of 0.8 (10⁸ cfu/ml). Subsequently, 200 μl of each lactobacilli culture, containing the vectors for short- or long-anchor protein versions, were washed three times in PBS by centrifugation (10,000×g for 15 min) before resuspension in 100 μl of PBS. An equal amount of mouse anti-E-tag antibody (Amersham Bioscience) diluted 1/200 was added and the samples were incubated on ice for 1 h. The washing procedure in PBS was repeated and the samples were resuspended in 100 μl of PBS and mixed with 100 μl cy2-labeled donkey anti-mouse antibodies (Jackson Immunoresearch Laboratories) (final dilution 1/200) and BD liquid counting beads (BD Biosciences) and incubated on ice for 30 min. After washing, the samples were resuspended in one ml of PBS and analysed in a FACSCalibur (BD Biosciences) machine for absolute quantitation of lactobacilli counts per volume.

Cell Separation and Co-Culture Conditions

To this end heparinised peripheral venous blood was obtained from patients with an established diagnosis of CVID or IgAD, according to the criteria of the European Society for Immunodeficiency Diseases (ESID), “Diagnostic criteria for PID”, http://www.esid.org/workingparty.php?party=3&sub=2&id=73; accessed on Oct. 12, 2008. PBMC were isolated by Ficoll separation and stored in liquid nitrogen until use, according to a method described by Kreher C R et al. (2003) J Immunol Methods 278 (1-2) 79-93. Subsequently, PBMC from CVID patients were co-cultured for 7 days with lactobacilli expressing surface-anchored CD40L, IL-21 cleaved, “Chim-hIL-21/4”, IL-21/IL-2 hybrid or IL-21/IL-4 hybrid protein in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂. The ratio between lactobacilli and PBMC was 10:1. PBMC from patients with IgAD were stimulated for 7 days with either 50 ng/ml of IL-21 cleaved, “Chim-hIL-21/4”, IL-21/IL-2 hybrid, or IL-21/IL-4 hybrid in combination with 2 μg/ml anti-CD40 mAb (Mabtech AB, Stockholm, Sweden) in Iscove's Modified Dulbecco's medium (IMDM) with 1% L-Alanyl-L-Glutamine, HEPES, 1% Penicillin-Streptomycin (Invitrogen Corporation, Carlsbad, Calif., USA), and 10% heat-inactivated foetal bovine serum (PAA Laboratories GmbH, Pasching, Germany) at 37° C. in the presence of 5% CO₂.

ELISPOT Assay and Determination of Immunoglobulin Amount

In order to determine the amount of immunoglobulines, an ELISPOT assay was performed. In particular, MultiScreenHTS Filter Plates (Millipore Corp., Bedford, Mass., USA) were pre-wet with 30% ethanol, rinsed three times with sterile PBS (sPBS) and coated overnight at 4° C. with either polyclonal rabbit anti-human IgG or IgA capture antibody (DAKO) diluted in sPBS at 10 μg/ml. After washing, plates were blocked for 3 hrs with sPBS containing 1% bovine serum albumin (SIGMA, St. Louis, Mo., USA). Cultured PBMC were plated at 2.5×10⁵ cells/well in cell culture medium as described above and incubated at 37° C. for 20 hrs in the presence of 5% CO₂. Thereafter, the plates were washed six times using PBS containing 0.01% Tween20 (PBS-Tween, SIGMA). Detection antibodies goat anti-human IgG-ALP (Mabtech AB) and goat anti-human IgA-ALP (SouthernBiotech) were diluted in PBS containing 0.5% bovine serum albumin and added at a final concentrations of 2 μg/ml. After overnight incubation at 4° C., the plates were washed six times with PBS-Tween. Spot development was carried out using the BCIP/NBT Liquid Substrate System (SIGMA). ELISPOT plate analysis and subsequent enumeration of cell counts and immunoglobulin amount was performed on the AID EliSpot 04 HR Reader using appropriate AID reader software, release 4.0 (Autoimmun Diagnostika GmbH, Strassberg, Germany). The immunoglobulin amount in FIGS. 15, 16 and 17 is measured in a virtual unit that is equivalent to the surface in (0.01 mm)² multiplied by the intensity of a particular spot. Single-well photos were shown FIGS. 15, 16 and 17 that depict the results of ELISPOT assays, whilst every dark spot represents a single IgA- or IgG-producing B cell.

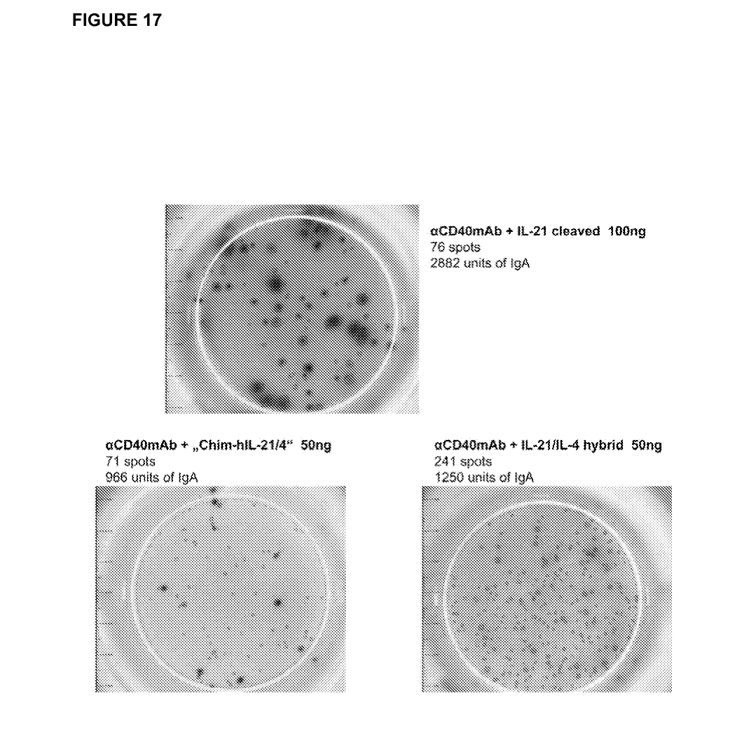

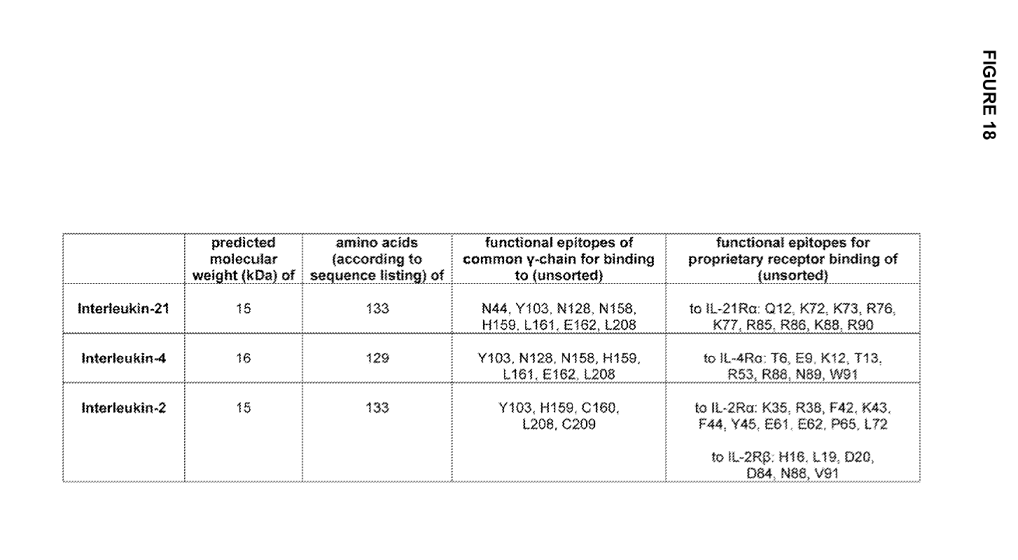

In FIG. 15 it is shown that lactobacilli containing surface-anchored CD40L and IL-21/IL-2 hybrid protein or IL-21/IL-4 hybrid protein induce a considerably greater amount of secreted IgA and IgA from PBMC of patients with CVID than was induced by lactobacilli containing surface-anchored CD40L and mature cleaved IL-21 or “Chim-hIL-21/4” protein. This clearly shows the suitability of lactobacilli expressing surface IL-21 variants to restore immunoglobulin production in patients with primary humoral immunodeficiency diseases. FIG. 16 shows the induction of IgA production of PBMC from patients with IgAD by lactobacilli surface-expressing IL-21 variants and by addition of soluble anti-CD40 antibody. Herein, lactobacilli containing surface-anchored IL-21/IL-2 hybrid protein or IL-21/IL-4 hybrid protein induced a considerably greater amount of secreted IgA than was induced by lactobacilli containing surface-anchored mature cleaved IL-21 or “Chim-hIL-21/4” protein. This demonstrates the suitability of lactobacilli expressing surface IL-21 variants to restore immunoglobulin production in patients with primary humoral immunodeficiency diseases. FIG. 17 shows that IL-21/IL-4 variant protein—in contrast to mature cleaved IL-21 or “Chim-hIL-21/4” protein—systemically induces a multiplicity of cells from patients with IgAD to produce IgA, typifying its mode of action.

### Example 13

### Designing and Testing of IL-21 Variants with the Functional Ability of Overlapping Receptor-Interaction

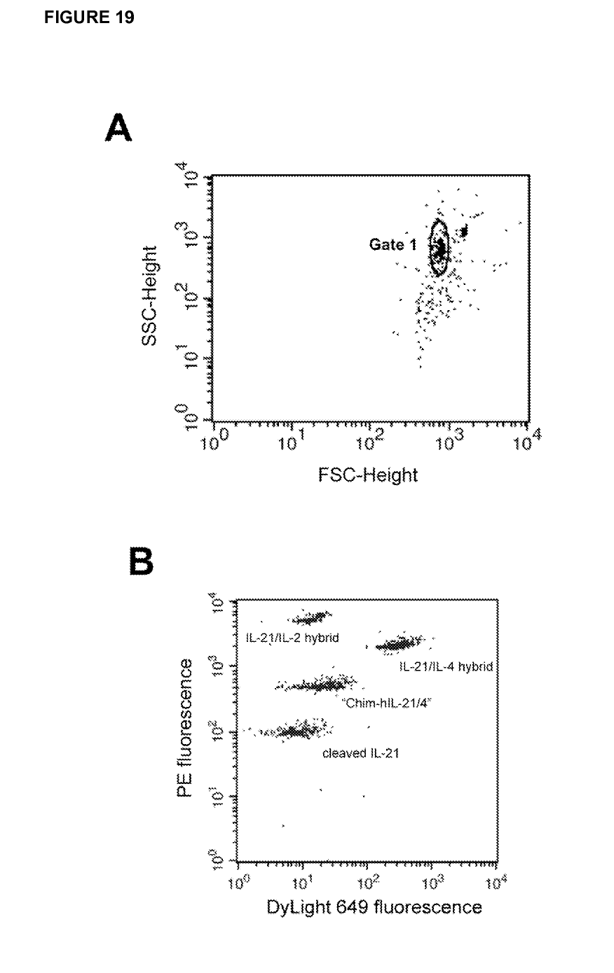

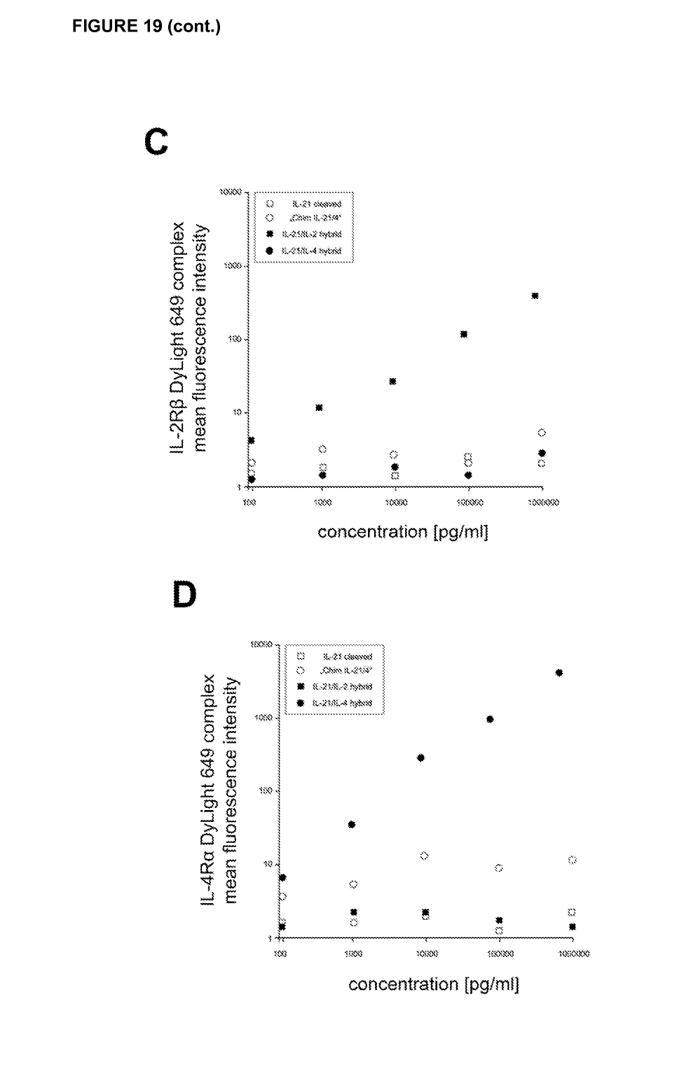

In the following experiment the calculation procedures forming the basis for the design of IL-21 variants with overlapping receptor binding abilities are depicted and synthetic, in-vitro expressed IL-21 variants are tested for their ability to bind proprietary IL-2Rβ and IL-4Rα proteins (see, for example, FIGS. 18 and 19).

Designing of IL-21 Variant Proteins with Overlapping Receptor Binding Abilities

The structural properties of human Interleukin-21, Interleukin-4 and Interleukin-2 (as specified in SEQ ID: 1, SEQ ID: 2, and SEQ ID: 3, respectively) can be compared and analyzed using the Accelrys Discovery Studio Visualizer (Accelrys Software Inc.). Upon structural- and sequence-alignment, there exists a significant structural homology between these cytokines, exemplified by (i) their spatial pattern consisting of a four-helix-bundle, (ii) a comparable quantity of residues and molecular weight, and (iii) similarities in residues involved in the binding to the common γ-chain (see FIGS. 1 and 18). To transduce any intracytoplasmic signals, IL-21, IL-4 and IL-2 require the common γ-chain and specific receptor subunits (IL-21Rα, IL-4Rα, IL-2Rα and IL-2Rβ). The interaction between IL-21, IL-4, and IL-2 and their specific receptor subunits and the common γ-chain can be analyzed by binding free energy calculations using the Accelrys Discovery Studio Visualizer and macromolecular structures of the receptor molecules accessible from the NCBI database (NCBI Computational Biology Branch). Hereupon, the common γ-chain, although indispensable for specific receptor activation, is shown to be a promiscuous binding protein that shares several epitopes important for binding to both IL-21, IL-4, and IL-2 (see FIG. 18), allowing alteration of residues within IL-21 variant proteins not critically important for common γ-chain interaction, thus retaining common γ-chain binding ability of the designed IL-21 variant proteins whilst featuring overlapping proprietary receptor binding effects. Residues important for the interaction between IL-21, IL-4, and IL-2 and their specific receptor subunits were calculated in the same manner and are shown in FIG. 18. In order to design IL-21 variant proteins with overlapping receptor binding abilities, regions close to the identified functional epitopes for proprietary receptor binding were compared using ligand interaction calculations in the Accelrys Discovery Studio Visualizer with the aim to identify structurally interchangeable regions of IL-21, IL-4-, and IL-2. Thereby, the region surrounding amino acid Gln¹² in IL-21 (position numbering according to SEQ ID NO: 1) was found to be structurally conserved, suggesting its interchangeability between IL-21, IL-4, and IL-2. An example is shown in the IL-21/IL-4 hybrid protein depicted in SEQ ID NO: 8, where the functionally important Glycine epitope is now present at position Gln¹¹. Moreover, in the IL-21/IL-4 hybrid protein of SEQ ID NO: 8 a region of IL-4, which is important for the formation of the interface for IL-4Rα interaction (Q⁷¹-S⁹⁸ of SEQ ID NO: 2), has been exchanged with a region formerly exclusively forming an interface for IL-21Rα binding, resulting in a structural assembly that allows both IL-4Rα and IL-21Rα interaction. In contrast thereto, a chimeric IL21-1L4 protein termed “Chim-hIL-21/4” which is described in a publication by Kent Bondensgaard (Bondensgaard et al. (2007) J Biol Chem. 282, 23326-36; shown in SEQ ID NO: 9) does not seem to be able to form a binding interface for IL-4Rα. Several regions were found not to be structurally conserved, which in term basically affects the receptor binding properties of resulting hybrid proteins when those regions are interchanged between IL-21, IL-4, and IL-2. An example is shown in the IL-21/IL-2 hybrid protein of SEQ ID NO: 7, where a region important for IL-2Rβ binding was successfully fused to a region formerly co-participating in IL-21Rα interaction.

Preparation of Plasmid DNA from IL-21 Variants

To this end, synthetic genes of IL-21 variants were assembled from synthetic oligonucleotides and PCR products according to the DNA sequences as set forth in SEQ ID NO: 31, SEQ ID NO: 32, SEQ ID NO: 33, and SEQ ID NO: 34; wherein SEQ ID NO:31 represents the cleaved version of IL-21 with the modification of a N-terminal hexa histidine-tag followed by a Factor Xa Protease recognition site. SEQ ID NO:32 refers to a protein described in a publication by Kent Bondensgaard (Bondensgaard, K., et al. (2007) J Biol Chem. 282, 23326-36) and termed “Chim-hIL-21/4”, that was also modified at the N-terminus with a hexa histidine-tag followed by a Factor Xa Protease recognition site; the original protein sequence of “Chim-hIL-21/4” has been indicated as SEQ ID NO: 9 in the sequence listing, and the corresponding DNA sequence has been indicated as SEQ ID NO: 16 in the sequence listing. Similarly, both IL-21 variants SEQ ID NO: 33 and SEQ ID NO: 34 were modified at the N-terminus with a hexa histidine-tag followed by a Factor Xa Protease recognition site. Synthetic genes of IL-21 variants were sent out for cloning into pET45b(+) (ampR) vectors (Novagen, Gibbstown, N.J., USA) using XbaI and XhoI restriction sites (imaGenes, Berlin, Germany). The final construct was verified by sequencing and the sequence congruence within the used restriction sites and the originally desired DNA sequences was 100% (imaGenes, Berlin, Germany). The plasmid DNA was purified from transformed bacteria using the Pure Yield™ Plasmid Midiprep System (Promega, Madison, Wis., USA) according to the manufacturer's instructions, and concentration was determined by UV spectroscopy.

In Vitro Synthesis and Purification of IL-21 Variant Proteins

E. coli-based in vitro synthesis of IL-21 variant proteins was done using the EasyXpress Protein Synthesis Mini Kit (Qiagen, Hilden, Germany) according to the manufacturer's instructions. Subsequently, the final reaction volume was subjected to Ni-NTA based purification for 6× His-tagged proteins under native conditions using Ni-NTA spin columns (Qiagen, Hilden, Germany). The hexa histidine-tag was cleaved from purified IL-21 variants using a Factor Xa Protease (Qiagen, Hilden, Germany) and removed from the reaction volume by either standard SDS page electrophoresis or another round of Ni-NTA spin column passage. The concentration of the cleaved IL-21 variant proteins was determined by standard Bradford protein assay (Bradford, M. (1976), Anal. Biochem. 72, 248-254) and adjusted to a concentration of 1 mg/ml protein in phosphate buffered saline (PBS) containing 0.5% bovine serum albumine (BSA).

Bead-Based Immunoassay for Detection of Receptor-Interaction

A cytometric bead array was designed to test IL-21 variant proteins for their ability to bind to the human Interleukin-21 receptor proprietary subunit (IL-21Rα), the human Interleukin-4 receptor alpha subunit (IL-4-binding subunit), the human Interleukin-2 receptor alpha (CD25), or the human Interleukin-2 receptor beta (CD122). Different color-coded 7.5 μm polystyrene beads (CBA Functional Beads, BD Biosciences) were covalently linked to both goat-anti human IL-21 polyclonal antibody and rabbit anti-human IL-21 polyclonal antibody (both from Abcam plc) using sulfo-SMCC chemistry (Functional Bead Conjugation Buffer Set, BD Biosciences). Both goat anti-human IL-21 and rabbit anti-human IL-21 antibodies were initially tested using a Western blot technique to confirm suitable detection of the IL-21 variant proteins with the predicted size. The success of antibody-to-bead conjugation was verified using PE Goat anti-Rabbit IgG Detector (BD Biosciences). To detect the receptor interaction of bead-captured IL-21 variants with the Interleukin-21 receptor proprietary subunit, the Interleukin-4 receptor alpha subunit, the Interleukin-2 receptor alpha or the Interleukin-2 receptor beta, appropriate receptor-Fc-chimera proteins were coupled with a Protein-A-fluorochrome complex suitable for flow cytometric evaluation. Therefore, Recombinant Human IL-21 R Subunit Fc Chimera protein (R&D Systems), Recombinant Human IL-4 Rα Fc Chimera protein (R&D Systems), Recombinant Human IL-2 Rα Fc Chimera protein (R&D Systems) or IL2 Receptor beta protein (Fc Chimera Active, Abcam plc) was reconstituted in Protein A IgG Binding Buffer (Thermo Fisher Scientific) and coupled to DyLight 649 Conjugated Protein A (Rockland Immunochemicals) according to the manufacturers' instructions, respectively. 50 μl of antibody-coated beads and serial dilutions of IL-21 variant proteins, with final concentrations ranging from 100 pg/ml to 1 μg/ml, were incubated together with the fluorescence conjugated receptor-chimera-Protein-A complexes in excess in 96-well plates for 3 h on a titer plate shaker. Afterwards, the plates were washed 2 times with 200 μl of CBA Wash Buffer (BD Biosciences) by centrifugation at 200×g for 5 minutes. The samples were carefully resuspended in 300 μl of CBA Wash Buffer and were immediately subjected to analysis. Bead particles were discriminated on the basis of size and fluorescence (see FIGS. 19A and B) on a BD FACSCanto II system with BD FACSDiva software (BD Biosciences). The extent of interaction between IL-21 variant proteins and receptor chimera proteins was calculated upon DyLight 649 fluorescence (APC channel) versus initial IL-21 variant protein concentration using the BD FACSDiva software and presented on scatter plots build with Sigma Plot (Systat Software Inc., see FIGS. 19C and D).

IL-21 variants were designed upon the knowledge of functional epitopes and regions that are important for the interaction of IL-21, IL-4, and IL-2 with the common γ-chain and their specific receptors, as shown in FIG. 18. The simulation and analysis of ligand interaction shows that several regions within IL-21, IL-4, and IL-2 are essential to form the interface for interaction with their cognate receptors, and can be fused into IL-21 variant proteins in order to obtain proteins with overlapping receptor binding abilities. Such IL-21 variant proteins can be tested by a bead-based immunoassay in order to detect and calculate the extent of receptor-interaction, as shown in FIG. 19. FIG. 19A shows that protein-coated beads can be securely detected by flow cytometry based on forward- and side-scatter analysis. In FIG. 19B the PE fluorescence of the color-coded beads is presented versus DyLight 649 fluorescence (APC channel) borne by receptor-chimera complexes, demonstrating that the extent of bound receptor-chimera-complex can be detected and analyzed by flow cytometry. FIG. 19C compares the ability of cleaved IL-21, “Chim-hIL-21/4”, IL-21/IL-2 hybrid protein and IL-21/IL-4 hybrid protein to bind the IL-2Rβ subunit, clearly indicating that only the IL-21/IL-2 hybrid protein shows a concentration-dependence of IL-21Rβ interaction, as intended by the design of this protein. In FIG. 19D the ability of cleaved IL-21, “Chim-hIL-21/4”, IL-21/IL-2 hybrid protein and IL-21/IL-4 hybrid protein to bind the IL-4Rα subunit is compared between the tested proteins. Whereas the “Chim-hIL-21/4” protein only allows minor IL-4Rα binding, an IL-21/IL-4 hybrid protein according to the present invention shows a clear concentration-dependence of IL-4Rα interaction, as intended by the design of this type of protein.

1. An Interleukin-21 (IL-21) variant, wherein said IL-21 variant is capable of increasing the secretion of IgG and/or IgA antibodies in B cells and/or is capable of binding the IL-2 receptor complex and/or the IL-4 receptor complex, said IL-21 variant comprising stretches of amino acids of Interleukin-4 (IL-4) or Interleukin-2 (IL-2) in substitution of amino acids of IL-21 as defined in SEQ ID NO: 1.

2. The IL-21 variant of claim 1, wherein said variant comprises between about 10 to 60% of the helical portions of IL-4 as defined in SEQ ID NO: 2, and wherein said variant optionally also comprises interhelical portions of IL-4 as defined in SEQ ID NO: 2.

3. The IL-21 variant of claim 1, wherein said variant comprises between about 10 to 65% of the helical portions of IL-2 as defined in SEQ ID NO: 3, and wherein said variant optionally also comprises interhelical portions of IL-2 as defined in SEQ ID NO: 3.

4. A pharmaceutical composition comprising IL-21 and/or the IL-21 variant of any one of claims 1 to 3, and at least one compound selected from the group consisting of IgA inducing protein (IGIP), Syntenin-1, Galectin-1 and Galectin-3.

5. A pharmaceutical composition for the treatment of a primary humoral immunodeficiency disease, comprising IL-21 and/or the IL-21 variant of any one of claims 1 to 3, and at least one compound selected from the group consisting of IL-4, IL-2, IgA inducing protein (IGIP), Syntenin-1, Galectin-1 and Galectin-3.

6. The pharmaceutical composition of claim 5, wherein said composition comprises IL-21 and/or the IL-21 variant of any one of claims 1 to 3, IL-4 and IL-2.

7. Use of IL-21 and/or the IL-21 variant of any one of claims 1 to 3, and at least one compound selected from the group consisting of IL-4, IL-2, IgA inducing protein (IGIP), Syntenin-1, Galectin-1 and Galectin-3 for the preparation of a pharmaceutical composition for the treatment of a primary humoral immunodeficiency disease.

8. A kit for the treatment of a primary humoral immunodeficiency disease, comprising:
(i) IL-21 and/or the IL-21 variant of any one of claims 1 to 3; and
(ii) IL-4 and/or IL-2; and/or
(iii) IgA inducing protein (IGIP) and/or Syntenin-1 and/or Galectin-1 and/or Galectin-3;
and optionally at least one element selected from:
(iv) a stimulator of CD40 molecules, preferably an anti-CD40 antibody, a CD40 ligand (CD40L) or C4BP;
(v) a ligand of the tumor necrosis superfamily, preferably BAFF or LIGHT;
(vi) a polypeptide with human leukocyte interferon activity, preferably Interferon-α (IFN-α);
(vii) a vaccine protein antigen; and
(viii) a vaccine polysaccharide antigen.

9. The pharmaceutical composition of claim 4, 5 or 6, the use of claim 7, or the kit of claim 8, wherein the ratio between IL-21 or said IL-21 variant and IL-4 in said pharmaceutical composition or in said kit is between about 5:1 and 25:1, preferably about 20:1.

10. The pharmaceutical composition of claim 4, 5 or 6, the use of claim 7, or the kit of claim 8, wherein the ratio between IL-21 or said IL-21 variant and IL-2 in said pharmaceutical composition or in said kit is between about 5:1 and 20:1, preferably about 15:1.

11. The pharmaceutical composition of any one of claim 4, 5, 6, 9 or 10, or the use of any one of claim 7, 9 or 10, wherein said pharmaceutical composition further comprises at least one stimulator of CD40 molecules, preferably an anti-CD40 antibody, CD40 ligand (CD40L) or C4BP.

12. The pharmaceutical composition of any one of claims 4, 5, 6 and 9 to 11, or the use of any one of claims 7 and 9 to 11, wherein said pharmaceutical composition further comprises at least one ligand of the tumor necrosis factor superfamily, preferably BAFF or LIGHT, and/or at least one polypeptide with human leukocyte interferon activity, preferably Interferon-α (IFN-α).

13. The pharmaceutical composition of any one of claims 4, 5, 6 and 9 to 12, or the use of any one of claims 7 and 9 to 12, wherein said pharmaceutical composition further comprises at least one vaccine protein antigen and/or at least one vaccine polysaccharide antigen.

14. The kit of any one of claims 8 to 10, wherein the interim between the administration of the compound(s) of (i) and the compound(s) of (ii) is between about 1 minute and 12 hours.

15. The kit of any one of claims 8 to 10 and 14, wherein the interim between the administration of the compound(s) of (i) plus (ii), and any of the compounds (iii) to (vii) is between about 12 hours and 72 hours.

16. A live carrier expressing IL-21 or an IL-21 variant as defined in any one of claims 1 to 3 and at least one element selected from the group consisting of IL-4, IL-2, IgA inducing protein (IGIP), Syntenin-1, Galectin-1 and Galectin-3, optionally also expressing at least one element selected from:
(i) a stimulator of CD40 molecules, preferably an anti-CD40 antibody, a CD40 ligand (CD40L) or C4BP;
(ii) a ligand of the tumor necrosis superfamily, preferably BAFF or LIGHT;
(iii) a polypeptide with human leukocyte interferon activity, preferably Interferon-α (IFN-α); and
(iv) a vaccine protein antigen.

17. The live carrier of claim 16, wherein said live carrier is for the treatment of a primary humoral immunodeficiency disease.

18. The pharmaceutical composition of any one of claims 4, 5, 6 and 9 to 13, the use of any one of claims 7 and 9 to 13, the kit of any one of claim 8 to 10, 14 or 15, or the live carrier of claim 17, wherein said primary humoral immunodeficiency disease is a disease involving a reduction in the level of secreted IgG and/or IgA antibodies.

19. The pharmaceutical composition, use, kit or live carrier of claim 18, wherein said disease is selective deficiency of IgA (IgAD), common variable immunodeficiency (CVID), selective deficiency of IgG subclasses (IgGsD), immunodeficiency with increased IgM (hyper-IgM-syndrome) or X-linked agammaglobulinaemia.


SEQUENCE LISTING (34 sequences)

SEQ ID NO: 1 - protein, 133 aa, Homo sapiens [sequence omitted]

SEQ ID NO: 2 - protein, 129 aa, Homo sapiens [sequence omitted]

SEQ ID NO: 3 - protein, 133 aa, Homo sapiens [sequence omitted]

SEQ ID NO: 4 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 5 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 153 aa, Homo sapiens [sequence omitted]

SEQ ID NO: 7 - protein, 127 aa, Artificial sequence
  Artificial hybrid sequence between human IL-21 and IL-2
QGQDRHMIRMRQLIDIVDQLKNYVNDLVPEFLPAPEDVETNCEWSAFSCFQKAQLKSANTGNNERIINVSISNINVIVLELKGSETTFMCDSYEKKPPKEFLERFKSLLQKMIHQHLSSRTHGSEDS

SEQ ID NO: 8 - protein, 126 aa, Artificial Sequence
  Artificial hybrid sequence between human IL-21 and IL-4
QGQDRHMITLQEIIKTLNSLTEQKTLVPEFLPAPEDVETNCEWSAFSCFQKAQLKSAQQFHRHKQLIRFLKRLDRNLWGLAGLNSCPSCDSYEKKPPKEFLERFKSLLQKMIHQHLSSRTHGSEDS

SEQ ID NO: 9 - protein, 127 aa, Artificial Sequence
  Artificial hybrid sequence between human IL-21 and IL-4
QGQDRHMIRMRQLIDIVDQLKNYVNDLVPEFLPAPEDVETNCEWSAFSCFQKAQLKSANTGNNERIINVSIKKLKRNLWGLAGLNSCPSCDSYEKKPPKEFLERFKSLLQKMIHQHLSSRTHGSEDS

SEQ ID NO: 10 - protein, 134 aa, Artificial Sequence
  IL-21 with N-terminal methionine
MQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 11 - protein, 134 aa, Artificial Sequence
  IL-2 with N-terminal methionine
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 130 aa, Artificial Sequence
  IL-4 with N-terminal methionine
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 13 - DNA, 399 bp, Homo sapiens
cagggccaggatcgtcatatgattcgtatgcgccagctgattgatattgtggatcagctgaaaaactatgtgaacgatctggtgccggaatttctgccggcaccggaagatgtggaaaccaactgcgaatggagcgcgtttagctgctttcagaaagcgcagctgaaaagcgcgaacaccggcaacaacgaacgtattatcaacgtgagcatcaaaaaactgaaacgtaaaccgccgagcacgaacgcgggtcgtcgtcagaaacatcgtctgacctgcccgagctgcgatagctatgaaaaaaaaccgccgaaagaatttctggaacgctttaaaagcctgctgcagaaaatgattcatcagcatctgagcagccgtacccatggcagcgaagatagc

SEQ ID NO: 14 - DNA, 381 bp, Artificial Sequence
  Artificial hybrid sequence between human IL-21 and IL-2
cagggccaggatcgtcatatgattcgtatgcgccagctgattgatattgtggatcagctgaaaaactatgtgaacgatctggtgccggaatttctgccggcaccggaagatgtggaaaccaactgcgaatggagcgcgtttagctgctttcagaaagcgcagctgaaaagcgcgaacaccggcaacaacgaacgtattatcaacgtgagcatcagcaacattaacgtgattgtgctggaactgaaaggcagcgaaaccacctttatgtgcgatagctatgaaaaaaaaccgccgaaagaatttctggaacgctttaaaagcctgctgcagaaaatgattcatcagcatctgagcagccgtacccatggcagcgaagatagc

SEQ ID NO: 15 - DNA, 378 bp, Artificial Sequence
  Artificial hybrid sequence between human IL-21 and IL-4
cagggccaggatcgtcatatgattaccctgcaggaaattatcaaaaccctgaacagcctgaccgaacagaaaaccctggtgccggaatttctgccggcaccggaagatgtggaaaccaactgcgaatggagcgcgtttagctgctttcagaaagcgcagctgaaaagcgcgcagcagtttcatcgtcataaacagctgatccgttttctgaaacgtctggatcgtaacctgtggggcctggccggtctgaatagctgcccgagctgcgatagctatgaaaaaaaaccgccgaaagaatttctggaacgctttaaaagcctgctgcagaaaatgattcatcagcatctgagcagccgtacccatggcagcgaagatagc

SEQ ID NO: 16 - DNA, 381 bp, Artificial Sequence
  Artificial hybrid sequence between human IL-21 and IL-4
cagggccaggatcgtcatatgattcgtatgcgccagctgattgatattgtggatcagctgaaaaactatgtgaacgatctggtgccggaatttctgccggcaccggaagatgtggaaaccaactgcgaatggagcgcgtttagctgctttcagaaagcgcagctgaaaagcgcgaacaccggcaacaacgaacgtattatcaacgtgagcatcaaaaaactgaaacgtaacctgtggggcctggcgggcctgaacagctgcccgagctgcgatagctatgaaaaaaaaccgccgaaagaatttctggaacgctttaaaagcctgctgcagaaaatgattcatcagcatctgagcagccgtacccatggcagcgaagatagc

SEQ ID NO: 17 - DNA, 21 bp, Artificial Sequence
  Primer sequence
cctgggcatggagtcctgtgg

SEQ ID NO: 18 - DNA, 21 bp, Artificial Sequence
  Primer sequence
ctgtgttggcgtacaggtctt

SEQ ID NO: 19 - DNA, 20 bp, Artificial Sequence
  Primer sequence
cacaaactcttccatcaggc

SEQ ID NO: 20 - DNA, 20 bp, Artificial Sequence
  Primer sequence
catccccacccataacaatc

SEQ ID NO: 21 - DNA, 20 bp, Artificial Sequence
  Primer sequence
ctcagccaggaccaaggaca

SEQ ID NO: 22 - DNA, 20 bp, Artificial Sequence
  Primer sequence
accacgctgctgagggagta

SEQ ID NO: 23 - DNA, 20 bp, Artificial Sequence
  Primer sequence
aatctgccggggactgaaac

SEQ ID NO: 24 - DNA, 18 bp, Artificial Sequence
  Primer sequence
tgagtggacctgccatga

SEQ ID NO: 25 - DNA, 20 bp, Artificial Sequence
  Primer sequence
ctgggattcgtgtagtgctt

SEQ ID NO: 26 - DNA, 19 bp, Artificial Sequence
  Primer sequence
cgtctgtgcctgcatgacg

SEQ ID NO: 27 - DNA, 21 bp, Artificial Sequence
  Primer sequence
ggagaagacagaaacacagac

SEQ ID NO: 28 - DNA, 21 bp, Artificial Sequence
  Primer sequence
gtccaactgcaagttagatcc

SEQ ID NO: 29 - DNA, 21 bp, Artificial Sequence
  Primer sequence
gtcatagtcttcattcctgcc

SEQ ID NO: 30 - DNA, 21 bp, Artificial Sequence
  Primer sequence
cacccatttcttgaagtctcc

SEQ ID NO: 31 - DNA, 429 bp, Artificial Sequence
  IL-21 His-Tag fusion
catcatcatcatcatcatattgaaggccgtcagggccaggatcgtcatatgattcgtatgcgccagctgattgatattgtggatcagctgaaaaactatgtgaacgatctggtgccggaatttctgccggcaccggaagatgtggaaaccaactgcgaatggagcgcgtttagctgctttcagaaagcgcagctgaaaagcgcgaacaccggcaacaacgaacgtattatcaacgtgagcatcaaaaaactgaaacgtaaaccgccgagcacgaacgcgggtcgtcgtcagaaacatcgtctgacctgcccgagctgcgatagctatgaaaaaaaaccgccgaaagaatttctggaacgctttaaaagcctgctgcagaaaatgattcatcagcatctgagcagccgtacccatggcagcgaagatagc

SEQ ID NO: 32 - DNA, 411 bp, Artificial Sequence
  Artificial hybrid sequence between human IL-21 and IL-4 fused to Hexa-His Tag
catcatcatcatcatcatattgaaggccgtcagggccaggatcgtcatatgattcgtatgcgccagctgattgatattgtggatcagctgaaaaactatgtgaacgatctggtgccggaatttctgccggcaccggaagatgtggaaaccaactgcgaatggagcgcgtttagctgctttcagaaagcgcagctgaaaagcgcgaacaccggcaacaacgaacgtattatcaacgtgagcatcaaaaaactgaaacgtaacctgtggggcctggcgggcctgaacagctgcccgagctgcgatagctatgaaaaaaaaccgccgaaagaatttctggaacgctttaaaagcctgctgcagaaaatgattcatcagcatctgagcagccgtacccatggcagcgaagatagc

SEQ ID NO: 33 - DNA, 411 bp, Artificial Sequence
  Artificial hybrid sequence between human IL-21 and IL-2 fused to Hexa-His Tag
catcatcatcatcatcatattgaaggccgtcagggccaggatcgtcatatgattcgtatgcgccagctgattgatattgtggatcagctgaaaaactatgtgaacgatctggtgccggaatttctgccggcaccggaagatgtggaaaccaactgcgaatggagcgcgtttagctgctttcagaaagcgcagctgaaaagcgcgaacaccggcaacaacgaacgtattatcaacgtgagcatcagcaacattaacgtgattgtgctggaactgaaaggcagcgaaaccacctttatgtgcgatagctatgaaaaaaaaccgccgaaagaatttctggaacgctttaaaagcctgctgcagaaaatgattcatcagcatctgagcagccgtacccatggcagcgaagatagc

SEQ ID NO: 34 - DNA, 408 bp, Artificial Sequence
  Artificial hybrid sequence between human IL-21 and IL-4 fused to Hexa-His Tag
catcatcatcatcatcatattgaaggccgtcagggccaggatcgtcatatgattaccctgcaggaaattatcaaaaccctgaacagcctgaccgaacagaaaaccctggtgccggaatttctgccggcaccggaagatgtggaaaccaactgcgaatggagcgcgtttagctgctttcagaaagcgcagctgaaaagcgcgcagcagtttcatcgtcataaacagctgatccgttttctgaaacgtctggatcgtaacctgtggggcctggccggtctgaatagctgcccgagctgcgatagctatgaaaaaaaaccgccgaaagaatttctggaacgctttaaaagcctgctgcagaaaatgattcatcagcatctgagcagccgtacccatggcagcgaagatagc